(19) United States
(12) Patent Application Publication
(10) Pub. No.: US 2024/0391937 A1
(43) Pub. Date: Nov. 28, 2024

(54) BICYCLIC DERIVATIVE PARP INHIBITOR AND USE THEREOF

(71) Applicant: XIZANG HAISCO PHARMACEUTICAL CO., LTD., Lhoka, Tibet (CN)

(72) Inventors: Yao LI, Shannan City, Tibet (CN); Wenjing WANG, Shannan City, Tibet (CN); Lei CHEN, Shannan City, Tibet (CN); Peng KANG, Shannan City, Tibet (CN); Chao FU, Shannan City, Tibet (CN); Fengkai CHENG, Shannan City, Tibet (CN); Pingming TANG, Shannan City, Tibet (CN); Yan YU, Shannan City, Tibet (CN); Chen ZHANG, Shannan City, Tibet (CN); Pangke YAN, Shannan City, Tibet (CN)

(21) Appl. No.: 18/697,419

(22) PCT Filed: Sep. 30, 2022
(86) PCT No.: PCT/CN2022/123423

§ 371 Date: Mar. 29, 2024

(30) Foreign Application Priority Data
Sep. 30, 2021 (CN) 202111163555.4
May 14, 2022 (CN) 202210525022.4
May 25, 2022 (CN) 202210578890.9
Jun. 20, 2022 (CN) 202210694483.4
Jul. 4, 2022 (CN) 202210786346.3

(51) Int. Cl. C07D 519/00; A61K 31/496; A61K 31/4985; A61P 35/00; C07D 471/04

(52) U.S. Cl. CPC C07D 519/00; A61K 31/496; A61K 31/4985; A61P 35/00; C07D 471/04

(57) ABSTRACT

Disclosed are a compound as shown in formula (I), a stereoisomer, pharmaceutically acceptable salt, solvate, cocrystal or deuterated compound thereof, or a pharmaceutical composition containing same, and the use thereof as a PARP-1 inhibitor in the preparation of a drug for treating related diseases, wherein the definition of each group in formula (I) is as defined in the description.

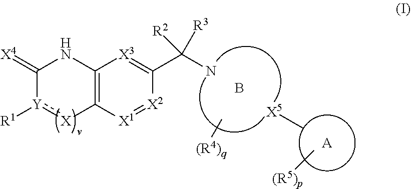

### TECHNICAL FIELD

The present invention belongs to the field of drugs, and in particular relates to a small molecule compound having a PARP-1 inhibitory activity, or a stereoisomer, pharmaceutically acceptable salt, solvate, cocrystal or deuterated material thereof, and the use thereof in the preparation of a drug for treating related diseases.

### BACKGROUND ART

Approximately 5% of breast cancer patients are associated with germline mutations in the BRCA1/2 genes (3% in the BRCA1 gene and 2% in the BRCA2 gene). Most breast cancers caused by BRCA1 mutations are triple-negative breast cancers (70%), while BRCA2 mutations are more likely to cause estrogen receptor-positive breast cancers (70%). The BRCA1/2 genes are tumor suppressor genes and play an important role in DNA damage repair, normal cell growth, etc. Mutations in the genes can inhibit the normal repair ability after DNA damage and cause homologous recombination deficiency (HRD), that is, loss of BRCA function or mutation or loss of function in other homologous recombination-related genes, making repair of DNA double-strand breaks impossible through homologous recombination repair (HRR), eventually leading to cancer.

Poly(ADP-ribose) polymerase (PARP) is a DNA repair enzyme that plays a key role in the DNA repair pathway. PARP is activated by DNA damage and breakage. As a molecular sensor of DNA damage, it has the function of identifying and binding to the DNA break location, thereby activating and catalyzing the polyADP ribosylation of the receptor protein and participating in the DNA repair process. PARP plays a key role in the process of DNA single-strand base excision and repair. In HRD tumor cells, DNA double-strand breaks cannot be repaired, and PARP inhibitors block the single-strand repair, resulting in a “synthetic lethal” effect, leading to tumor cell death.

PARP inhibitors have a “trapping” effect on the PARP protein, causing the PARP protein that binds to damaged DNA to be trapped on the DNA, directly causing other DNA repair proteins to be unable to bind, eventually leading to cell death. At present, several PARP inhibitors have been successfully developed, such as olaparib, rucapalib and niraparib. However, adverse reactions limit their ability to be used in combination with chemotherapy drugs. This may be related to the lack of selectivity of marketed PARP inhibitors against the PARP family. These side effects include intestinal toxicity caused by tankyrase inhibition and hematological toxicity caused by PARP-2 inhibition. Therefore, it is of great clinical significance to develop highly selective PARP-1 inhibitors and reduce the toxic and side effects associated with non-selective PARP inhibitors.

### SUMMARY OF THE INVENTION

The objective of the present invention is to provide a compound that inhibits PARP-1, or a stereoisomer, solvate, deuterated compound, or pharmaceutically acceptable salt thereof, and the medical application thereof. The compound has the advantages of good efficacy, low toxic and side effects, high safety, strong selectivity, good pharmacokinetics, high bioavailability, and no inhibition to CYP enzymes.

The present invention relates to a compound as shown in formula (I), (II), (II-a), (II-b), (III), (III-a), (III-b), (III-c), (IV), (IV-a), (IV-b), (IV-c), (V), (VI), (VII), (VIII) or (IX), or a stereoisomer, solvate, deuterated compound, or pharmaceutically acceptable salt thereof,

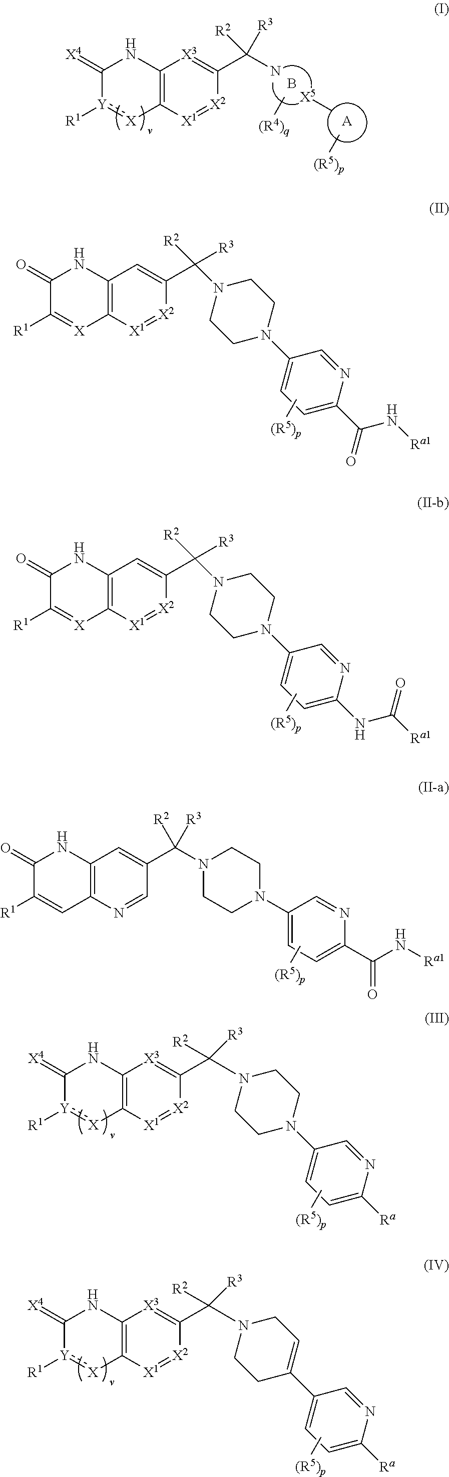

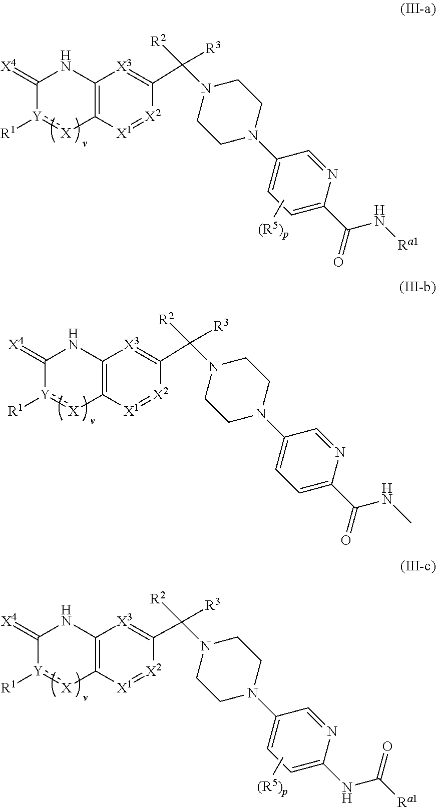

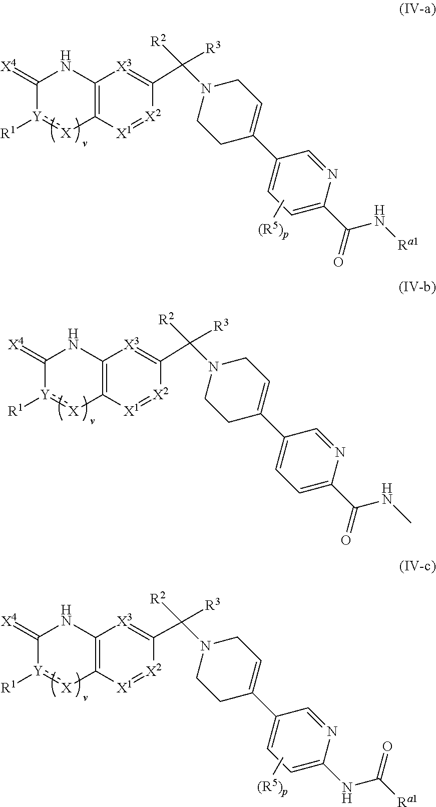

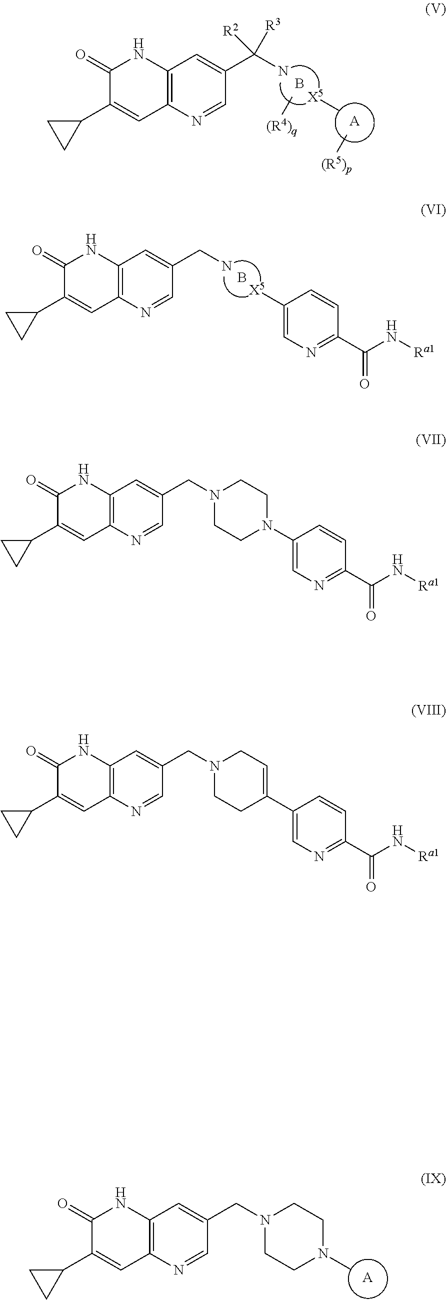

wherein X is selected from CR^{x}, C(R^{x})₂, O, N or NR^{x};
Y is selected from N, C or CH; in some embodiments, Y is selected from N or C; in some embodiments, Y is selected from C;
[symbol] represents a single bond or a double bond;
v is selected from 1, 2 or 3; in some embodiments, v is selected from 1 or 2; in some embodiments, v is selected from 1;
X¹, X² and X³ are each independently selected from N or CR^{x}; in some embodiments, X¹ is selected from N, and X² and X³ are selected from CR^{x}; in some embodiments, X¹, X² and X³ are selected from N; in some embodiments, X¹ is selected from N, X² is selected from N, and X³ is selected from CR^{x}; in some embodiments, X¹ is selected from N, X² is selected from CR^{x}, and X³ is selected from N; in some embodiments, X¹, X² and X³ are selected from CR^{x}; in some embodiments, X¹ is selected from CR^{x}, and X² and X³ are selected from N; in some embodiments, X¹ is selected from CR^{x}, X² is selected from N, and X³ is selected from CR^{x}; in some embodiments, X¹ is selected from CR^{x}, X² is selected from CR^{x}, and X³ is selected from N;
provided that when [symbol] represents a double bond, and v is selected from 1, X, X¹, X² and X³ are not all selected from CR^{x};
X⁴ is selected from O or S; in some embodiments, X⁴ is selected from O; in some embodiments, X⁴ is selected from S;
X⁵ is selected from N or CR^{x}; in some embodiments, X⁵ is selected from N or CH; in some embodiments, X⁵ is selected from N; in some embodiments, X⁵ is selected from CR^{x}; in some embodiments, X⁵ is selected from CH;
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O; in some embodiments, each R^{x} is independently selected from H, D, halogen, cyano, amino, hydroxyl, C₁₋₄ alkyl, halo C₁₋₄ alkyl, halo C₁₋₄ alkoxy, deuterated C₁₋₄ alkyl, deuterated C₁₋₄ alkoxy, C₁₋₄ alkoxy, C₂₋₄ alkenyl, C₂₋₄ alkynyl, C₁₋₂ alkyl-O—C₁₋₂ alkyl, —(CH₂)ᵣ—C₃₋₆ monocyclic cycloalkyl, —(CH₂)ᵣ—C₅₋₇ bicyclic spiro cycloalkyl, —(CH₂)ᵣ-(4- to 6-membered heterocycloalkyl), or —(CH₂)ᵣ-(7- to 9-membered bicyclic spiro heterocycloalkyl); or two R^{x} on the same carbon atom together form ═O; in some embodiments, each R^{x} is independently selected from H, D, F, Cl, cyano, amino, hydroxyl, C₁₋₂ alkyl, halo C₁₋₂ alkyl, deuterated C₁₋₂ alkyl, C₁₋₂ alkoxy, —(CH₂)ᵣ—C₃₋₄ monocyclic cycloalkyl, —(CH₂)ᵣ—C₅₋₆ bicyclic spiro cycloalkyl, —(CH₂)ᵣ-(4- to 5-membered heterocycloalkyl), or —(CH₂)ᵣ-(7- to 8-membered bicyclic spiro heterocycloalkyl); or two R^{x} on the same carbon atom together form ═O; in some embodiments, each R^{x} is independently selected from H, D, F, Cl, cyano, hydroxyl, C₁₋₂ alkyl, halo C₁₋₂ alkyl, or deuterated C₁₋₂ alkyl; or two R^{x} on the same carbon atom together form ═O; in some embodiments, each R^{x} is independently selected from H, D, C₁₋₂ alkyl, halo C₁₋₂ alkyl, or deuterated C₁₋₂ alkyl; or two R^{x} on the same carbon atom together form ═O; in some embodiments, each R^{x} is independently selected from H or D, or two R^{x} on the same carbon atom together form ═O; in some embodiments, each R^{x} is independently selected from H or D; R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1, 2 or 3 groups selected from D, halogen, cyano, amino, hydroxyl, C₁₋₆ alkyl or C₁₋₆ alkoxy; in some embodiments, R¹ is selected from halogen, nitro, cyano, amino, hydroxyl, —SF₅, C₁₋₄ alkyl, C₁₋₄ alkoxy, C₂₋₄ alkenyl, C₂₋₄ alkynyl, C₁₋₂ alkyl-O—C₁₋₂ alkyl, —(CH₂)ᵣ—C₃₋₆ monocyclic cycloalkyl, —(CH₂)ᵣ—C₅₋₇ bicyclic spiro cycloalkyl, —(CH₂)ᵣ-(4- to 6-membered heterocycloalkyl), or —(CH₂)ᵣ-(6- to 9-membered bicyclic spiro heterocycloalkyl), wherein the alkyl, alkoxy, alkenyl, alkynyl, cycloalkyl, or heterocycloalkyl is optionally further substituted with 1, 2 or 3 groups selected from D, halogen, cyano, amino, hydroxyl, C₁₋₂ alkyl, or C₁₋₂ alkoxy; in some embodiments, R¹ is selected from F, Cl, cyano, amino, hydroxyl, C₁₋₂ alkyl, C₁₋₂ alkoxy, C₂₋₃ alkenyl, C₁₋₂ alkyl-O—C₁₋₂ alkyl, —(CH₂)ᵣ—C₃₋₄ monocyclic cycloalkyl, —(CH₂)ᵣ—C₅₋₇ bicyclic spiro cycloalkyl, —(CH₂)ᵣ-(4- to 5-membered heterocycloalkyl), or —(CH₂)ᵣ-(6- to 8-membered bicyclic spiro heterocycloalkyl), wherein the alkyl, alkoxy, alkenyl, alkynyl, cycloalkyl, or heterocycloalkyl is optionally further substituted with 1, 2 or 3 groups selected from D, halogen, cyano, amino, hydroxyl, C₁₋₂ alkyl, or C₁₋₂ alkoxy; in some embodiments, R¹ is selected from cyano, C₁₋₂ alkyl, C₂₋₃ alkenyl, C₁₋₂ alkyl-O—C₁₋₂ alkyl, C₃₋₄ monocyclic cycloalkyl, or 4- to 5-membered heterocycloalkyl, wherein the alkyl, alkenyl, cycloalkyl, or heterocycloalkyl is optionally further substituted with 1, 2 or 3 groups selected from D, F, Cl, cyano, amino, hydroxyl, C₁₋₂ alkyl, or C₁₋₂ alkoxy; in some embodiments, R¹ is selected from cyano, C₁₋₂ alkyl, C₂₋₃ alkenyl, C₁₋₂ alkyl-O—C₁₋₂ alkyl, or C₃₋₄ monocyclic cycloalkyl;
each r is independently selected from 0, 1, 2 or 3; in some embodiments, each r is independently selected from 0 or 1; in some embodiments, r is selected from 0;
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
in some embodiments, R² and R³ are each independently selected from H, D, halogen, cyano, amino, hydroxyl, C₁₋₂ alkyl-O—C₁₋₂ alkyl, hydroxy C₁₋₄ alkyl, C₁₋₄ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form C₃₋₅ cycloalkyl or 4- to 5-membered heterocycloalkyl; in some embodiments, R² and R³ are each independently selected from H, D, halogen, cyano, amino, hydroxyl, C₁₋₂ alkyl-O—C₁₋₂ alkyl, hydroxy C₁₋₂ alkyl, C₁₋₂ alkoxy, halo C₁₋₂ alkyl, halo C₁₋₂ alkoxy, deuterated C₁₋₂ alkyl, deuterated C₁₋₂ alkoxy or C₁₋₂ alkyl; or R² and R³ together with the carbon atom to which they are attached form C₃₋₄ cycloalkyl or 4-membered heterocycloalkyl; in some embodiments, R² and R³ are each independently selected from H, D, F, hydroxyl, halo C₁₋₂ alkyl, deuterated C₁₋₂ alkyl or C₁₋₂ alkyl; or R² and R³ together with the carbon atom to which they are attached form C₃₋₄ cycloalkyl; in some embodiments, R² and R³ are each independently selected from H, D, or C₁₋₂ alkyl; or R² and R³ together with the carbon atom to which they are attached form C₃₋₄ cycloalkyl; in some embodiments, R² and R³ are each independently selected from H, D, or C₁₋₂ alkyl; or R² and R³ together with the carbon atom to which they are attached form C₃₋₄ cycloalkyl; in some embodiments, R² and R³ are each independently selected from H or D; in some embodiments, R² and R³ are each independently selected from H, D, halogen, cyano, amino, hydroxyl, C₁₋₂ alkyl-O—C₁₋₂ alkyl, hydroxy C₁₋₂ alkyl, C₁₋₂ alkoxy, halo C₁₋₂ alkyl, halo C₁₋₂ alkoxy, deuterated C₁₋₂ alkyl, deuterated C₁₋₂ alkoxy or C₁₋₂ alkyl; or R² and R³ together with the carbon atom to which they are attached form C₃₋₅ cycloalkyl or 4- to 5-membered heterocycloalkyl;
each R⁴ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; or two R⁴ on the same carbon atom together with the carbon atom to which they are attached form ═O; in some embodiments, each R⁴ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy; or two R⁴ on the same carbon atom together with the carbon atom to which they are attached form ═O; in some embodiments, each R⁴ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy; or two R⁴ on the same carbon atom together with the carbon atom to which they are attached form ═O; in some embodiments, each R⁴ is independently selected from D, F, Cl, cyano, amino, hydroxyl, C₁₋₂ alkyl, C₁₋₂ alkoxy, halo C₁₋₂ alkyl, halo C₁₋₂ alkoxy, deuterated C₁₋₂ alkyl, or deuterated C₁₋₂ alkoxy; or two R⁴ on the same carbon atom together with the carbon atom to which they are attached form ═O; in some embodiments, each R⁴ is independently selected from D, F, Cl, methyl, ethyl, methoxy, ethoxy, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃; or two R⁴ on the same carbon atom together with the carbon atom to which they are attached form ═O;
each R⁵ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; in some embodiments, each R⁵ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy; in some embodiments, each R⁵ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy; in some embodiments, each R⁵ is independently selected from D, F, Cl, cyano, amino, hydroxyl, C₁₋₂ alkyl, C₁₋₂ alkoxy, halo C₁₋₂ alkyl, halo C₁₋₂ alkoxy, deuterated C₁₋₂ alkyl, or deuterated C₁₋₂ alkoxy; in some embodiments, each R⁵ is independently selected from D, F, Cl, C₁₋₂ alkyl, halo C₁₋₂ alkyl, or deuterated C₁₋₂ alkyl; in some embodiments, each R⁵ is independently selected from D, F, Cl, methyl, ethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃; q is selected from 0, 1, 2 or 3; in some embodiments, q is selected from 0, 1 or 2; in some embodiments, q is selected from 0 or 1; in some embodiments, q is selected from 0; p is selected from 0, 1, 2 or 3; in some embodiments, p is selected from 0, 1 or 2; in some embodiments, p is selected from 0 or 1; in some embodiments, p is selected from 0; ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms; in some embodiments, ring B is 5-membered saturated monocyclic heterocycloalkane containing 1-2 nitrogen atoms, 6-membered saturated monocyclic heterocycloalkane containing 1-2 nitrogen atoms, 6-membered saturated bridged heterocycle containing 1, 2, 3 or 4 nitrogen atoms, 7-membered saturated bridged heterocycle containing 1, 2, 3 or 4 nitrogen atoms, 8-membered saturated bridged heterocycle containing 1, 2, 3 or 4 nitrogen atoms, 8-membered saturated fused heterocycle containing 1, 2, 3 or 4 nitrogen atoms, 9-membered saturated fused heterocycle containing 1, 2, 3 or 4 nitrogen atoms, 10-membered saturated fused heterocycle containing 1, 2, 3 or 4 nitrogen atoms, 7-membered saturated spiro heterocycle containing 1, 2, 3 or 4 nitrogen atoms, 8-membered saturated spiro heterocycle containing 1, 2, 3 or 4 nitrogen atoms, 9-membered saturated spiro heterocycle containing 1, 2, 3 or 4 nitrogen atoms, 10-membered saturated spiro heterocycle containing 1, 2, 3 or 4 nitrogen atoms, or 11-membered saturated spiro heterocycle containing 1, 2, 3 or 4 nitrogen atoms; in some embodiments, ring B is 6-membered saturated monocyclic heterocycloalkane containing 1-2 nitrogen atoms, 6-membered saturated bridged heterocycle containing 1, 2, 3 or 4 nitrogen atoms, 7-membered saturated bridged heterocycle containing 1, 2, 3 or 4 nitrogen atoms, 8-membered saturated bridged heterocycle containing 1, 2, 3 or 4 nitrogen atoms, 7-membered saturated spiro heterocycle containing 1, 2, 3 or 4 nitrogen atoms, 9-membered saturated spiro heterocycle containing 1, 2, 3 or 4 nitrogen atoms, or 11-membered saturated spiro heterocycle containing 1, 2, 3 or 4 nitrogen atoms; in some embodiments, ring B is 6-membered saturated monocyclic heterocycloalkane containing 1-2 nitrogen atoms; in some embodiments, ring B is selected from

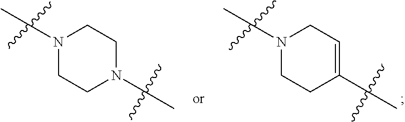 [SMILES: *N1CCN(*)CC1 ; *C1=CCN(*)CC1]

ring A is selected from 5- to 6-membered monocyclic heteroaromatic ring containing 1, 2, 3, 4 or 5 nitrogen, oxygen or sulfur atoms, wherein the heteroaromatic ring is further substituted with 1-3 substituents selected from Rₐ; in some embodiments, ring A is selected from 5-membered monocyclic heteroaromatic ring containing 1, 2, 3, 4 or 5 nitrogen, oxygen or sulfur atoms, or 6-membered monocyclic heteroaromatic ring containing 1, 2, 3, 4 or 5 nitrogen, oxygen or sulfur atoms, wherein the heteroaromatic ring is further substituted with 1, 2 or 3 substituents selected from Rₐ; in some embodiments, ring A is selected from 6-membered monocyclic heteroaromatic ring containing 1, 2, 3, 4 or 5 nitrogen, oxygen or sulfur atoms, wherein the heteroaromatic ring is further substituted with 1 substituent selected from Rₐ; in some embodiments, ring A is selected from 6-membered monocyclic heteroaromatic ring containing 1 or 2 nitrogen atoms, wherein the heteroaromatic ring is further substituted with 1 substituent selected from Rₐ; in some embodiments, ring A is selected from 5-membered monocyclic heteroaromatic ring containing 2, 3, 4 or 5 nitrogen, oxygen or sulfur atoms, wherein the heteroaromatic ring is further substituted with 1 substituent selected from Rₐ; or ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b}; in some embodiments, ring A is selected from 8- to 10-membered bicyclic fused heteroaromatic ring containing 1, 2, 3, 4 or 5 nitrogen, oxygen or sulfur atoms, or 8- to 10-membered bicyclic fused aromatic ring, wherein the heteroaromatic ring or aromatic ring is optionally further substituted with 1, 2 or 3 substituents selected from R_{b}; in some embodiments, ring A is selected from 8-membered bicyclic fused heteroaromatic ring containing 1, 2, 3, 4 or 5 nitrogen, oxygen or sulfur atoms, 9-membered bicyclic fused heteroaromatic ring containing 1, 2, 3, 4 or 5 nitrogen, oxygen or sulfur atoms, 10-membered bicyclic fused heteroaromatic ring containing 1, 2, 3, 4 or 5 nitrogen, oxygen or sulfur atoms, 8-membered bicyclic fused aromatic ring, 9-membered bicyclic fused aromatic ring, or 10-membered bicyclic fused aromatic ring, wherein the heteroaromatic ring or aromatic ring is optionally further substituted with 1 substituent selected from R_{b}; in some embodiments, ring A is selected from 8-membered bicyclic fused heteroaromatic ring containing 1, 2, 3, 4 or 5 nitrogen, oxygen or sulfur atoms, 9-membered bicyclic fused heteroaromatic ring containing 1, 2, 3, 4 or 5 nitrogen, oxygen or sulfur atoms, 10-membered bicyclic fused heteroaromatic ring containing 1, 2, 3, 4 or 5 nitrogen, oxygen or sulfur atoms, 8-membered bicyclic fused aromatic ring, 9-membered bicyclic fused aromatic ring, or 10-membered bicyclic fused aromatic ring, wherein the heteroaromatic ring or aromatic ring is optionally further substituted with 1 substituent selected from R_{b};
in some embodiments, ring A is selected from

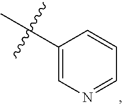 [SMILES: *c1cccnc1]

which is further substituted with 1-3 substituents selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b};
in some embodiments, ring A is selected from 8- to 10-membered bicyclic heteroaromatic ring containing 1-5 nitrogen, oxygen or sulfur atoms, wherein the heteroaromatic ring is optionally further substituted with 1, 2 or 3 substituents selected from —C(O)N(R^{a1})₂, —NHC₁₋₂ alkyl, —N(C₁₋₂ alkyl)₂, C₁₋₂ alkyl, ═O, C₁₋₂ alkyl, C₃₋₅ cycloalkyl, halo C₁₋₂ alkyl, halo C₁₋₂ alkoxy, deuterated C₁₋₂ alkyl, or deuterated C₁₋₂ alkoxy;
Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy; in some embodiments, Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5-membered monocyclic heteroaryl containing 1, 2, 3, 4 or 5 nitrogen, oxygen or sulfur atoms, 6-membered monocyclic heteroaryl containing 1, 2, 3, 4 or 5 nitrogen, oxygen or sulfur atoms, 4- to 7-membered monocyclic heterocycloalkyl containing 1, 2, 3 or 4 nitrogen, oxygen or sulfur atoms, or 3- to 7-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHC₁₋₂ alkyl, —N(C₁₋₂ alkyl)₂, C₁₋₂ alkyl, halo C₁₋₂ alkyl, C₁₋₂ alkoxy, halo C₁₋₂ alkoxy, deuterated C₁₋₂ alkyl, or deuterated C₁₋₂ alkoxy; in some embodiments, Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R³¹)₂, 5-membered monocyclic heteroaryl containing 1, 2, 3 or 4 nitrogen, oxygen or sulfur atoms, 4-membered monocyclic heterocycloalkyl containing 1 or 2 nitrogen, oxygen or sulfur atoms, 5-membered monocyclic heterocycloalkyl containing 1 or 2 nitrogen, oxygen or sulfur atoms, 6-membered monocyclic heterocycloalkyl containing 1 or 2 nitrogen, oxygen or sulfur atoms, 4-membered monocyclic cycloalkyl, 5-membered monocyclic cycloalkyl, or 6-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHCH₃, —NHCH₂CH₃, —N(CH₃)₂, methyl, ethyl, methoxy, ethoxy, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —OCHF₂, —OCH₂F, —OCF₃, —OCH₂CH₂F, —OCH₂CHF₂, —OCH₂CF₃, —OCHFCH₂F, —OCHFCHF₂, —OCHFCF₃, —OCF₂CH₂F, —OCF₂CHF₂, —OCF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, —CD₂CD₃, —OCHD₂, —OCH₂D, —OCD₃, —OCH₂CH₂D, —OCH₂CHD₂, —OCH₂CD₃, —OCHDCH₂D, —OCHDCHD₂, —OCHDCD₃, —OCD₂CH₂D, —OCD₂CHD₂, or —OCD₂CD₃; in some embodiments, Rₐ is selected from —C(O)N(R^{a1})₂, —NR^{a1}C(O)R^{a1} or —C(O)R^{a1};
R_{b} is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy; in some embodiments, R_{b} is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₂ alkyl, —N(C₁₋₂ alkyl)₂, C₁₋₂ alkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl or deuterated C₁₋₂ alkoxy; in some embodiments, R_{b} is selected from —C(O)N(R^{a1})₂, ═O, D, halogen, cyano, hydroxyl, amino, —NHC₁₋₂ alkyl, —N(C₁₋₂ alkyl)₂, C₁₋₂ alkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl or deuterated C₁₋₂ alkoxy; in some embodiments, R_{b} is selected from —C(O)N(R^{a1})₂, ═O, D, halogen, cyano, hydroxyl, amino, —NHC₁₋₂ alkyl, —N(C₁₋₂ alkyl)₂, C₁₋₂ alkyl, or halo C₁₋₂ alkyl; in some embodiments, R_{b} is selected from —C(O)N(R^{a1})₂, ═O, D, halogen, cyano, hydroxyl, amino, —NHCH₃, —NHCH₂CH₃, —N(CH₃)₂, methyl, ethyl, —CH₂F, —CHF₂, —CF₃, —CH₂CH₂F, —CH₂CHF₂, —CH₂CF₃, —CHFCH₂F, —CHFCHF₂, or —CHFCF₃; in some embodiments, R_{b} is selected from D, halogen, cyano, hydroxyl, amino, C₁₋₂ alkyl, C₃₋₅ cycloalkyl, 3- to 5-membered heterocycloalkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl, or deuterated C₁₋₂ alkoxy;

each R^{a1} is independently selected from H, D, C₁₋₆ alkyl, C₃₋₁₂ cycloalkyl, 3- to 12-membered heterocycloalkyl, 5- to 10-membered heteroaryl, C₁₋₆ alkoxy, C₁₋₆ alkyl-O—C₁₋₆ alkyl, C₁₋₆ alkyl-O—C₃₋₁₂ cycloalkyl, halo C₁₋₆ alkyl, halo C₁₋₆ alkoxy, deuterated C₁₋₆ alkyl, or deuterated C₁₋₆ alkoxy, wherein the alkyl, cycloalkyl, heterocycloalkyl, or heteroaryl is optionally further substituted with 1-3 substituents selected from D, halogen, cyano, hydroxyl, amino, C₁₋₆ alkyl, halo C₁₋₆ alkyl, deuterated C₁₋₆ alkyl, C₃₋₆ cycloalkyl or phenyl; in some embodiments, each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₄ cycloalkyl or phenyl; in some embodiments, each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₆ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, F, Cl, cyano, hydroxyl, amino, C₁₋₂ alkyl, halo C₁₋₂ alkyl, deuterated C₁₋₂ alkyl, C₃₋₄ cycloalkyl or phenyl; in some embodiments, R^{a1} is selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 6-membered heteroaryl, C₁₋₄ alkoxy, C₁₋₂ alkyl-O—C₁₋₂ alkyl, C₁₋₂ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₅ cycloalkyl or phenyl; in some embodiments, each R^{a1} is independently selected from methyl, ethyl, propyl, isopropyl, tert-butyl, cyclopropyl, cyclobutyl, methoxymethyl, ethoxymethyl, methoxyethyl, ethoxyethyl, cyclopropyloxyethyl, difluorocyclopropyloxyethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, —CH₂CH₂CF₃,

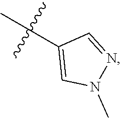 [SMILES: *c1cnn(C)c1]

—CH₂-cyclopropyl,

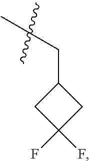 [SMILES: *CC1CC(F)(F)C1]

fluorocyclopropyl, difluorocyclopropyl, difluorocyclobutyl,

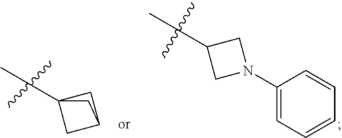 [SMILES: *C12CC(C1)C2 ; *C1CN(c2ccccc2)C1]

in some embodiments, each R^{a1} is independently selected from H, D, C₁₋₆ alkyl, C₃₋₁₂ cycloalkyl, 3- to 12-membered heterocycloalkyl, 5- to 10-membered heteroaryl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the alkyl, cycloalkyl, heterocycloalkyl, or heteroaryl is optionally further substituted with 1-3 substituents selected from D, halogen, cyano, hydroxyl, amino, C₁₋₆ alkyl, or C₃₋₆ cycloalkyl; in some embodiments, each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, or C₃₋₄ cycloalkyl; in some embodiments, each R^{a1} is independently selected from H, D, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy; in some embodiments, each R^{a1} is independently selected from H, D, C₁₋₄ alkyl, C₃₋₆ monocyclic cycloalkyl, C₅₋₁₁ bicyclic spiro cycloalkyl, C₆₋₈ bicyclic bridged cycloalkyl, C₇₋₁₀ bicyclic fused cycloalkyl, 4- to 6-membered heterocycloalkyl, 6- to 9-membered bicyclic spiro heterocycloalkyl, C₆₋₈ bicyclic bridged heterocycloalkyl, C₇₋₁₀ bicyclic fused heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy; in some embodiments, each R^{a1} is independently selected from C₁₋₄ alkyl, C₃₋₆ monocyclic cycloalkyl, C₅₋₇ bicyclic spiro cycloalkyl, 4- to 6-membered heterocycloalkyl, C₁₋₂ alkyl-O—C₁₋₂ alkyl, halo C₁₋₄ alkyl, or deuterated C₁₋₄ alkyl; in some embodiments, each R^{a1} is independently selected from H, D, C₁₋₂ alkyl, C₃₋₅ cycloalkyl, 3- to 5-membered heterocycloalkyl, 5- to 6-membered heteroaryl, halo C₁₋₂ alkyl, halo C₁₋₂ alkoxy, deuterated C₁₋₂ alkyl, or deuterated C₁₋₂ alkoxy, wherein the cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, or C₃₋₄ cycloalkyl; in some embodiments, each R^{a1} is independently selected from C₁₋₄ alkyl, C₃₋₅ cycloalkyl, or 5- to 6-membered heteroaryl, wherein the alkyl, cycloalkyl, or heteroaryl is optionally further substituted with 1, 2 or 3 substituents selected from D, F, Cl, cyano, hydroxyl, amino, C₁₋₂ alkyl, halo C₁₋₂ alkyl, or deuterated C₁₋₂ alkyl; in some embodiments, each R^{a1} is independently selected from C₁₋₄ alkyl, C₃₋₄ monocyclic cycloalkyl, C₁₋₂ alkyl-O—C₁₋₂ alkyl, halo C₁₋₄ alkyl, or deuterated C₁₋₄ alkyl; in some embodiments, each R^{a1} is independently selected from methyl, ethyl, propyl, isopropyl, tert-butyl, cyclopropyl, cyclobutyl, methoxymethyl, ethoxymethyl, methoxyethyl, ethoxyethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, or —CH₂CH₂CF₃;
unless otherwise specified, the above-mentioned heterocycloalkane, heterocycloalkyl, heteroaryl, or heteroaromatic ring contains 1, 2, 3, 4 or 5 heteroatoms selected from nitrogen, oxygen or sulfur; in some embodiments, the heterocycloalkane, heterocycloalkyl, heteroaryl, or heteroaromatic ring contains 1, 2, 3 or 4 heteroatoms selected from nitrogen, oxygen or sulfur; the heterocycloalkane, heterocycloalkyl, heteroaryl, or heteroaromatic ring contains 1, 2 or 3 heteroatoms selected from nitrogen, oxygen or sulfur; the heterocycloalkane, heterocycloalkyl, heteroaryl, or heteroaromatic ring contains 1 or 2 heteroatoms selected from nitrogen, oxygen or sulfur.

The present invention provides a compound as shown in formula (I), or a stereoisomer, solvate, deuterated compound, or pharmaceutically acceptable salt thereof, wherein in some embodiments, when [symbol] represents a single bond, X¹, X² and X³ are all selected from CR^{x}, and v is selected from 1, X is selected from C(R^{x})₂ or NR^{x}.

The present invention provides a compound as shown in formula (I), or a stereoisomer, solvate, deuterated compound, or pharmaceutically acceptable salt thereof, wherein in some embodiments, the following conditions are met: (1) when [symbol] represents a single bond, X¹, X² and X³ are all selected from CR^{x}, and v is selected from 1, X is selected from C(R^{x})₂ or NR^{x}; (2) when

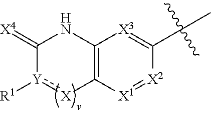

is selected from

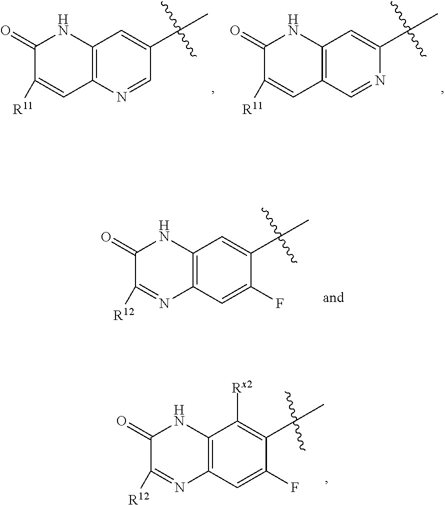

wherein R^{x2} is selected from H, C₁₋₆ alkyl or halogen, R¹² is selected from H, C₁₋₆ alkyl, halo C₁₋₆ alkyl, C₁₋₆ alkoxy or C₃₋₆ cycloalkyl, R¹¹ is selected from C₁₋₆ alkyl, and R² is selected from H or D, R³ is not selected from H or D.

The present invention provides a compound as shown in formula (I), or a stereoisomer, solvate, deuterated compound, or pharmaceutically acceptable salt thereof, wherein in some embodiments, the following conditions are met: (1) when [symbol] represents a single bond, X¹, X² and X³ are all selected from CR^{x}, and v is selected from 1, X is selected from C(R^{x})₂ or NR^{x}; (2) when [symbol] represents a double bond, and v is selected from 1, X, X¹, X² and X³ are not all selected from CR^{x}; (3) when R² is selected from H or D and R³ is selected from H or D, X⁴ is selected from O, X³ is selected from CH, [symbol] represents a double bond, v is selected from 1, and Y is selected from C, R¹ is not C₁₋₆ alkyl and halo C₁₋₆ alkyl.

The present invention provides a compound, or a stereoisomer, solvate, deuterated compound, or pharmaceutically acceptable salt thereof, wherein the selection of groups follows the rules of chemical bonding.

Specifically, the first technical solution of the present invention provides a compound as shown in formula (I), or a stereoisomer, solvate, deuterated compound, or pharmaceutically acceptable salt thereof,

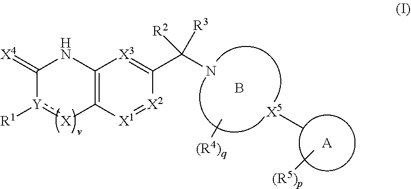

wherein X is selected from CR^{x}, C(R^{x})₂, O, N or NR^{x};
Y is selected from N, C or CH;
[symbol] represents a single bond or a double bond;
v is selected from 1, 2 or 3;
X¹, X² and X³ are each independently selected from N or CR^{x};
provided that when [symbol] represents a double bond, and v is selected from 1, X, X¹, X² and X³ are not all selected from CR^{x};
X⁴ is selected from O or S;
X⁵ is selected from N or CR^{x};
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1, 2 or 3;
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
each R⁴ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; or two R⁴ on the same carbon atom together with the carbon atom to which they are attached form ═O;
each R⁵ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy;
q is selected from 0, 1, 2 or 3; p is selected from 0, 1, 2 or 3;
ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms;
ring A is selected from 5- to 6-membered monocyclic heteroaromatic ring containing 1-5 nitrogen, oxygen or sulfur atoms, wherein the heteroaromatic ring is further substituted with 1-3 substituents selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b};
Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy;
R_{b} is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy;
each R^{a1} is independently selected from H, D, C₁₋₆ alkyl, C₃₋₁₂ cycloalkyl, 3- to 12-membered heterocycloalkyl, 5- to 10-membered heteroaryl, C₁₋₆ alkoxy, C₁₋₆ alkyl-O—C₁₋₆ alkyl, C₁₋₆ alkyl-O—C₃₋₁₂ cycloalkyl, halo C₁₋₆ alkyl, halo C₁₋₆ alkoxy, deuterated C₁₋₆ alkyl, or deuterated C₁₋₆ alkoxy, wherein the alkyl, cycloalkyl, heterocycloalkyl, or heteroaryl is optionally further substituted with 1-3 substituents selected from D, halogen, cyano, hydroxyl, amino, C₁₋₆ alkyl, halo C₁₋₆ alkyl, deuterated C₁₋₆ alkyl, C₃₋₆ cycloalkyl or phenyl;
provided that:
(1) when [symbol] represents a single bond, X¹, X² and X³ are all selected from CR^{x}, and v is selected from 1, X is selected from C(R^{x})₂ or NR^{x};
(2) when

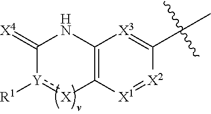

is selected from

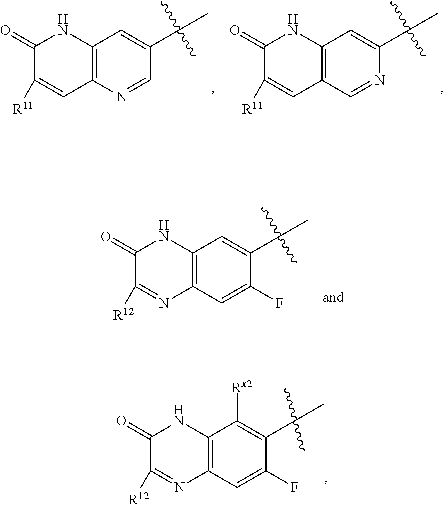

wherein R^{x2} is selected from H, C₁₋₆ alkyl or halogen, R¹² is selected from H, C₁₋₆ alkyl, halo C₁₋₆ alkyl, C₁₋₆ alkoxy or C₃₋₆ cycloalkyl, R¹¹ is selected from C₁₋₆ alkyl, and R² is selected from H or D, R³ is not selected from H or D;
unless otherwise specified, the above-mentioned heterocycloalkane, heterocycloalkyl, heteroaryl, or heteroaromatic ring contains 1-5 heteroatoms selected from nitrogen, oxygen or sulfur.

The present invention provides a compound as shown in formula (I), or a stereoisomer, solvate, deuterated compound, or pharmaceutically acceptable salt thereof, wherein
wherein X is selected from CR^{x}, C(R^{x})₂, O, N or NR^{x};
Y is selected from N, C or CH;
[symbol] represents a single bond or a double bond;
v is selected from 1, 2 or 3;
X¹, X² and X³ are each independently selected from N or CR^{x};
provided that when [symbol] represents a double bond, and v is selected from 1, X, X¹, X² and X³ are not all selected from CR^{x};
X⁴ is selected from O or S;
X⁵ is selected from N or CR^{x};
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1, 2 or 3;
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
each R⁴ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; or two R⁴ on the same carbon atom together with the carbon atom to which they are attached form ═O;
each R⁵ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy;
q is selected from 0, 1, 2 or 3; p is selected from 0, 1, 2 or 3;
ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms;
ring A is selected from 5- to 6-membered monocyclic heteroaromatic ring containing 1-5 nitrogen, oxygen or sulfur atoms, wherein the heteroaromatic ring is further substituted with 1-3 substituents selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b};
Rₐ is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl or deuterated C₁₋₆ alkoxy;
R_{b} is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy;
each R^{a1} is independently selected from H, D, C₁₋₆ alkyl, C₃₋₁₂ cycloalkyl, 3- to 12-membered heterocycloalkyl, 5- to 10-membered heteroaryl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the alkyl, cycloalkyl, heterocycloalkyl, or heteroaryl is optionally further substituted with 1-3 substituents selected from D, halogen, cyano, hydroxyl, amino, C₁₋₆ alkyl, or C₃₋₆ cycloalkyl; or each R^{a1} is independently selected from H, D, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy;
provided that when [symbol] represents a single bond, X¹, X² and X³ are all selected from CR^{x}, and v is selected from 1, X is selected from C(R^{x})₂ or NR^{x};
unless otherwise specified, the above-mentioned heterocycloalkane, heterocycloalkyl, heteroaryl, or heteroaromatic ring contains 1-5 heteroatoms selected from nitrogen, oxygen or sulfur.

The present invention provides a compound as shown in formula (I), or a stereoisomer, solvate, deuterated compound, or pharmaceutically acceptable salt thereof, wherein in some embodiments, the following conditions are also met: when

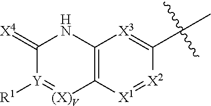

is selected from

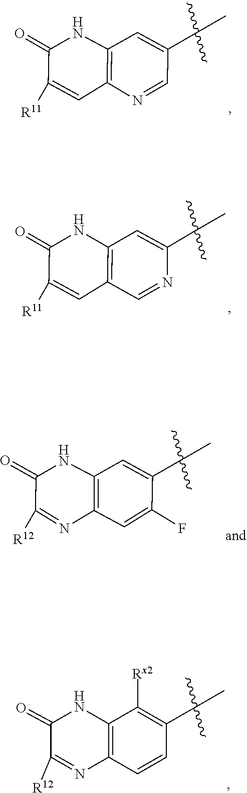

wherein R^{x2} is selected from H, C₁₋₆ alkyl or halogen, R¹² is selected from H, C₁₋₆ alkyl, halo C₁₋₆ alkyl, C₁₋₆ alkoxy or C₃₋₆ cycloalkyl, R¹¹ is selected from C₁₋₆ alkyl, and R² is selected from H or D, R³ is not selected from H or D.

The second technical solution of the present invention relates to the compound as shown in formula (I), or the stereoisomer, solvate, deuterated compound, or pharmaceutically acceptable salt thereof according to the present invention, wherein
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl; provided that when R² is selected from H or D, R³ is not selected from H or D;
other groups are consistent with those of any one of the preceding technical solutions.

The third technical solution of the present invention relates to the compound as shown in formula (I), or the stereoisomer, solvate, deuterated compound, or pharmaceutically acceptable salt thereof according to the present invention, having a structure of formula (II) or (II-a):

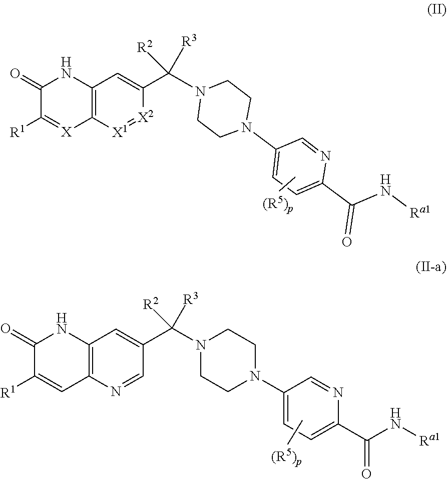

wherein X is selected from CR^{x} or N;
X¹ and X² are each independently selected from N or CR^{x};
p is selected from 0 or 1;
R^{x} is independently selected from H, D, halogen, cyano, amino, hydroxyl, C₁₋₂ alkyl, C₁₋₂ alkoxy, C₂₋₄ alkenyl, C₂₋₄ alkynyl, C₁₋₂ alkyl-O—C₁₋₂ alkyl, —(CH₂)ᵣ—C₃₋₅ cycloalkyl, —(CH₂)ᵣ—C₅₋₉ bicyclic spiro cycloalkyl, —(CH₂)ᵣ-(4- to 5-membered heterocycloalkyl), or —(CH₂)ᵣ-(5- to 9-membered bicyclic spiro heterocycloalkyl);
R¹ is selected from C₁₋₆ alkyl or halo C₁₋₆ alkyl;
R⁵ is selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, or C₃₋₄ cycloalkyl; or each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl or deuterated C₁₋₄ alkoxy;
R² and R³ are each independently selected from H, D, halogen, cyano, amino, hydroxyl, C₁₋₂ alkyl-O—C₁₋₂ alkyl, hydroxy C₁₋₃ alkyl, C₁₋₃ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form 3-membered cycloalkyl, 4-membered cycloalkyl, 5-membered cycloalkyl, 4-membered heterocycloalkyl, or 5-membered heterocycloalkyl, provided that when R² is selected from H or D, R³ is not selected from H or D;
other groups are consistent with those mentioned above.

The fourth technical solution of the present invention relates to the compound as shown in formula (I), or the stereoisomer, solvate, deuterated compound, or pharmaceutically acceptable salt thereof according to the present invention, having a structure of formula (II) or (II-b):

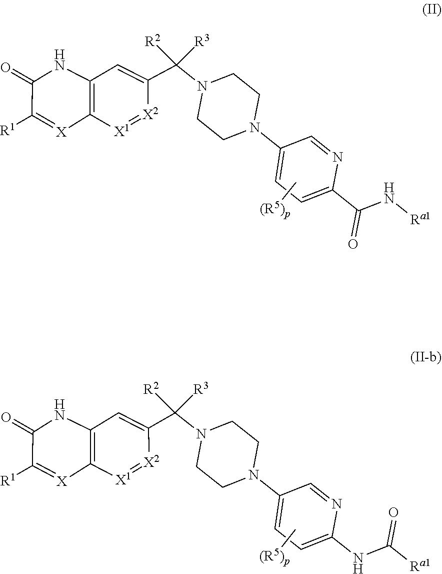

wherein X is selected from CR^{x} or N;
X¹ and X² are each independently selected from N or CR^{x};
p is selected from 0 or 1;
R^{x} is independently selected from H, D, halogen, cyano, amino, hydroxyl, C₁₋₂ alkyl, C₁₋₂ alkoxy, C₂₋₄ alkenyl, C₂₋₄ alkynyl, C₁₋₂ alkyl-O—C₁₋₂ alkyl, —(CH₂)ᵣ—C₃₋₅ cycloalkyl, —(CH₂)ᵣ—C₅₋₉ bicyclic spiro cycloalkyl, —(CH₂)ᵣ-(4- to 5-membered heterocycloalkyl), or —(CH₂)ᵣ-(5- to 9-membered bicyclic spiro heterocycloalkyl);
R¹ is selected from C₁₋₆ alkyl or halo C₁₋₆ alkyl;
R⁵ is selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₄ cycloalkyl or phenyl;
R² and R³ are each independently selected from H, D, halogen, cyano, amino, hydroxyl, C₁₋₂ alkyl-O—C₁₋₂ alkyl, hydroxy C₁₋₃ alkyl, C₁₋₃ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form 3-membered cycloalkyl, 4-membered cycloalkyl, 5-membered cycloalkyl, 4-membered heterocycloalkyl, or 5-membered heterocycloalkyl, provided that when R² is selected from H or D, R³ is not selected from H or D.

The fifth technical solution of the present invention relates to the compound as shown in formula (I), or the stereoisomer, solvate, deuterated compound, or pharmaceutically acceptable salt thereof according to the present invention, having a structure of formula (III), (IV), (III-a), (III-b), (III-c), (IV-a), (IV-b) or (IV-c):

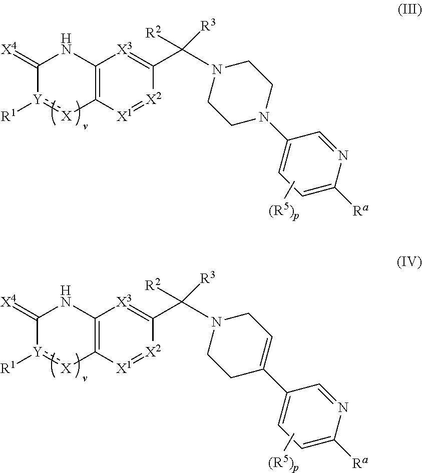

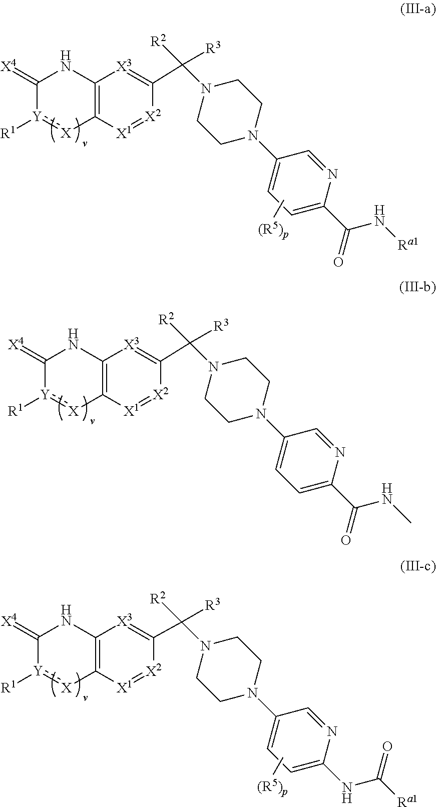

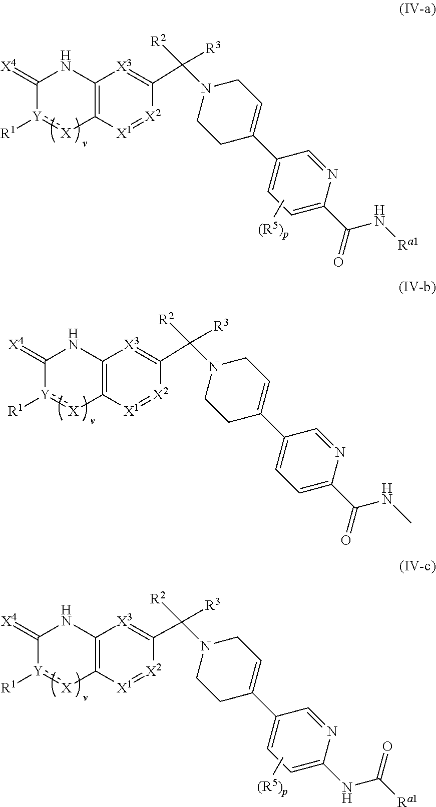

wherein X is selected from CR^{x}, C(R^{x})₂ or O;
Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-4 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₂ alkyl, —N(C₁₋₂ alkyl)₂, C₁₋₄ alkyl, halo C₁₋₄ alkyl, C₁₋₄ alkoxy, halo C₁₋₄ alkoxy, deuterated C₁₋₄ alkyl, or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₆ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, F, Cl, cyano, hydroxyl, amino, C₁₋₂ alkyl, halo C₁₋₂ alkyl, deuterated C₁₋₂ alkyl, C₃₋₄ cycloalkyl or phenyl;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1 or 2;
provided that when R² is selected from H or D and R³ is selected from H or D, X⁴ is selected from O, X³ is selected from CH, [symbol] represents a double bond, v is selected from 1, and Y is selected from C, R¹ is not C₁₋₆ alkyl and halo C₁₋₆ alkyl.

The sixth technical solution of the present invention relates to the compound as shown in formula (I), or the stereoisomer, solvate, deuterated compound, or pharmaceutically acceptable salt thereof according to the present invention, having a structure of formula (III), (III-a) or (III-b):

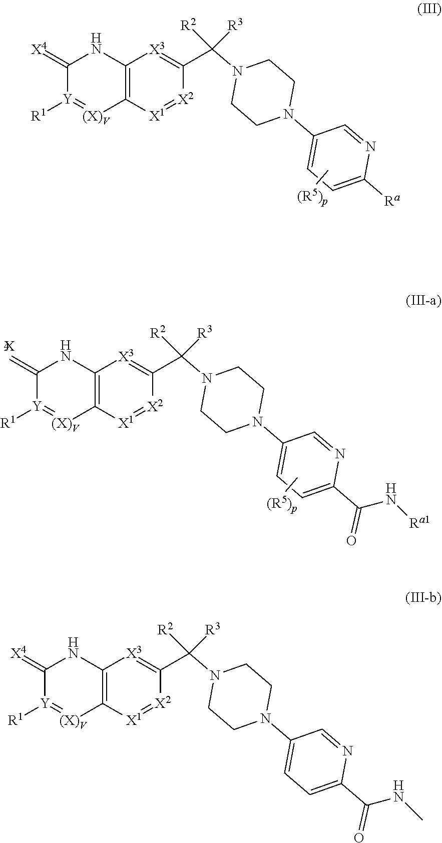

wherein X is selected from CR^{x}, C(R^{x})₂ or O;
Rₐ is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-4 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₂ alkyl, —N(C₁₋₂ alkyl)₂, C₁₋₄ alkyl, halo C₁₋₄ alkyl, C₁₋₄ alkoxy, halo C₁₋₄ alkoxy, deuterated C₁₋₄ alkyl or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, F, Cl, cyano, hydroxyl, amino, C₁₋₂ alkyl, or C₃₋₄ cycloalkyl; or each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl or deuterated C₁₋₄ alkoxy;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1 or 2;
provided that when R² is selected from H or D and R³ is selected from H or D, X⁴ is selected from O, X³ is selected from CH, [symbol] represents a double bond, v is selected from 1, and Y is selected from C, R¹ is not C₁₋₆ alkyl and halo C₁₋₆ alkyl;
other groups are consistent with those mentioned above.

The seventh technical solution of the present invention relates to the compound as shown in formula (I), (II), (II-a), (II-b), (III), (III-a), (III-b), (III-c), (IV), (IV-a), (IV-b) or (IV-c), or the stereoisomer, solvate, deuterated compound, or pharmaceutically acceptable salt thereof according to the present invention, wherein
X is selected from CR^{x}, C(R^{x})₂ or O;
v is selected from 1 or 2;
p is selected from 0 or 1;
each R is independently selected from H, D, halogen, cyano, amino, hydroxyl, C₁₋₄ alkyl, C₁₋₄ alkoxy, C₂₋₄ alkenyl, C₂₋₄ alkynyl, C₁₋₄ alkyl-O—C₁₋₄ alkyl, —(CH₂)-C₃₋₆ monocyclic cycloalkyl or —(CH₂)ᵣ-(4- to 6-membered monocyclic heterocycloalkyl); or two R^{x} on the same carbon atom together form ═O;
each R⁵ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy;
R^{a1} is selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 6-membered heteroaryl, C₁₋₄ alkoxy, C₁₋₂ alkyl-O—C₁₋₂ alkyl, C₁₋₂ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₅ cycloalkyl or phenyl; in some embodiments, R^{a1} is selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl or deuterated C₁₋₄ alkoxy;
R² and R³ are each independently selected from H, D, halogen, cyano, amino, hydroxyl, C₁₋₂ alkyl-O—C₁₋₂ alkyl, hydroxy C₁₋₃ alkyl, C₁₋₃ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form 3-membered cycloalkyl, 4-membered cycloalkyl, 5-membered cycloalkyl, 4-membered heterocycloalkyl, or 5-membered heterocycloalkyl;
the definitions of other groups are consistent with those mentioned above.

The eighth technical solution of the present invention relates to the compound as shown in formula (I), (II), (II-a), (II-b), (III), (III-a), (III-b), (III-c), (IV), (IV-a), (IV-b) or (IV-c), or the stereoisomer, solvate, deuterated compound, or pharmaceutically acceptable salt thereof according to the present invention, wherein
R¹ is selected from halogen, nitro, cyano, amino, hydroxyl, —SF₅, C₁₋₄ alkyl, C₁₋₄ alkoxy, C₂₋₄ alkenyl, C₂₋₄ alkynyl, C₁₋₄ alkyl-O—C₁₋₄ alkyl, —(CH₂)ᵣ—C₃₋₆ monocyclic cycloalkyl, —(CH₂)ᵣ—C₅₋₉ bicyclic spiro cycloalkyl, —(CH₂)ᵣ-(4- to 6-membered monocyclic heterocycloalkyl) or —(CH₂)ᵣ-(5- to 9-membered bicyclic spiro heterocycloalkyl), wherein the alkyl, alkoxy, alkenyl, alkynyl, cycloalkyl, or heterocycloalkyl is optionally further substituted with 1-3 groups selected from D, F, Cl, cyano, amino, hydroxyl, C₁₋₃ alkyl or C₁₋₃ alkoxy.

The ninth technical solution of the present invention relates to the compound as shown in formula (I), (II), (II-a), (II-b), (III), (III-a), (III-b), (III-c), (IV), (IV-a), (IV-b), (IV-c), (V), (VI), (VII), (VIII) or (IX), or the stereoisomer, solvate, deuterated compound, or pharmaceutically acceptable salt thereof according to the present invention, wherein

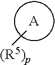

is selected from

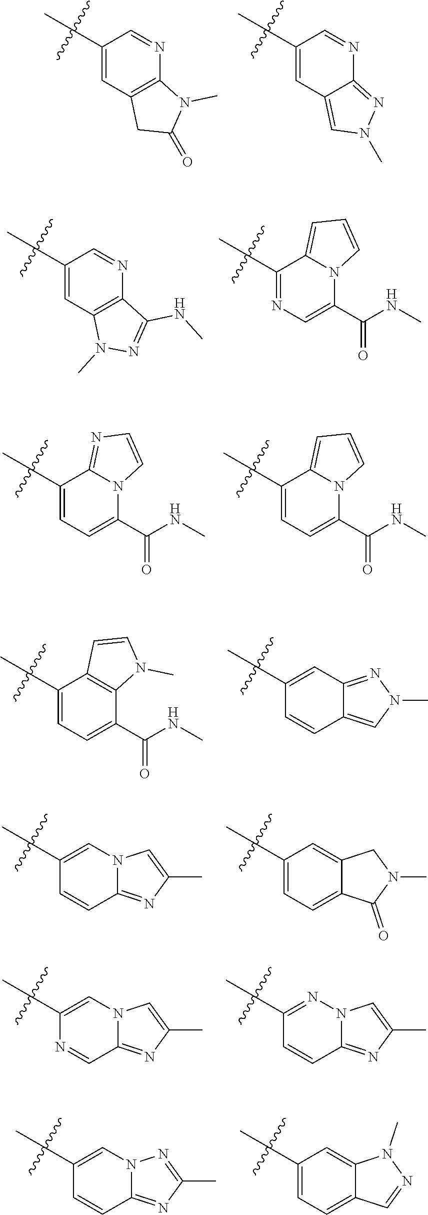

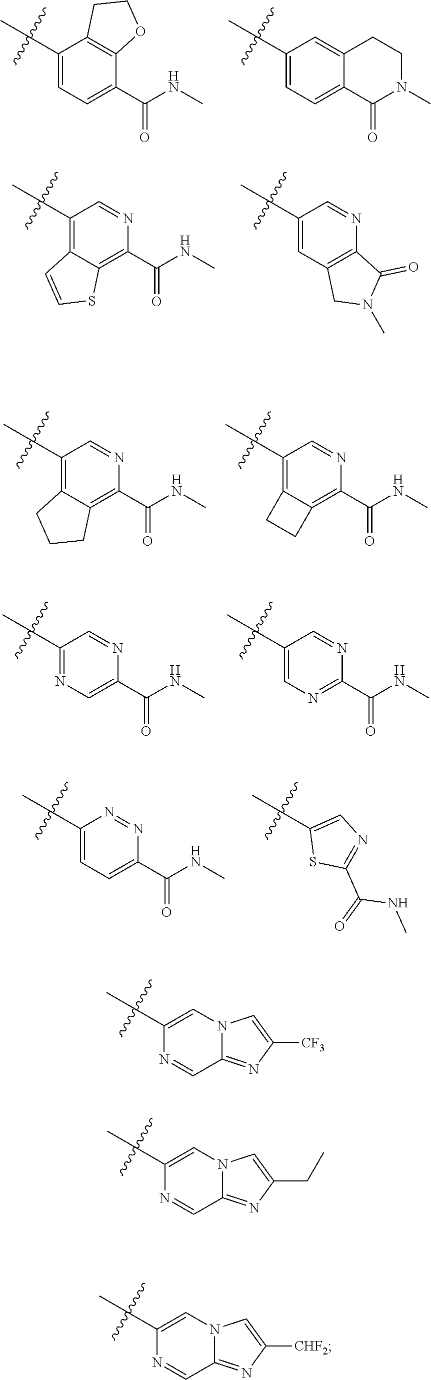

or

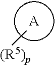

is selected from

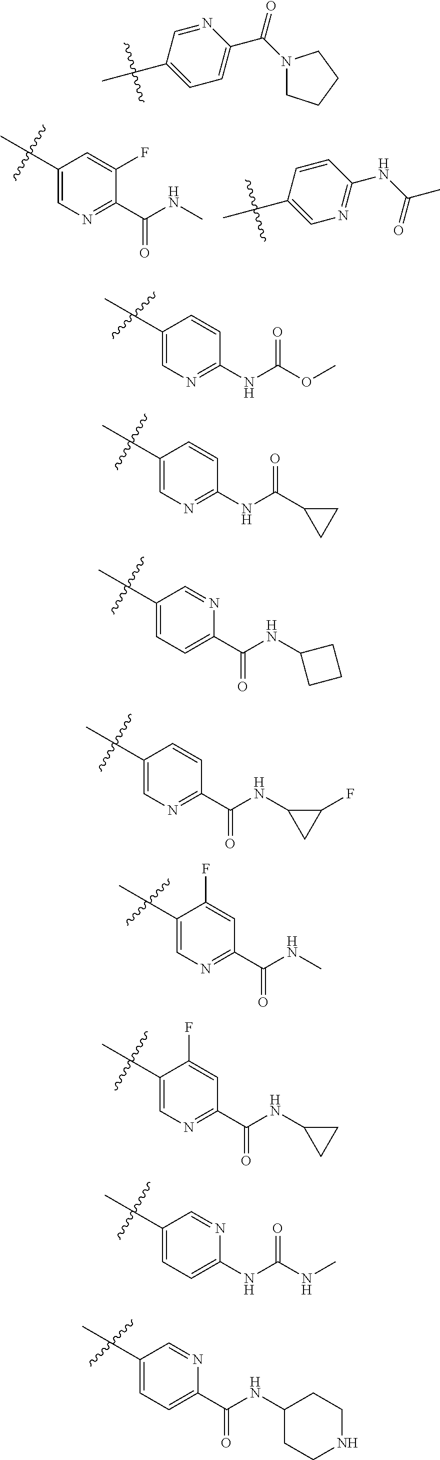

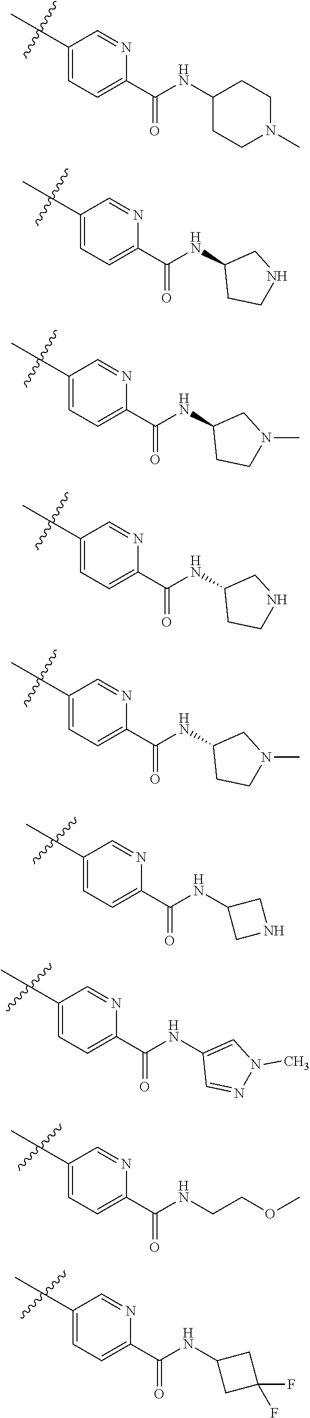

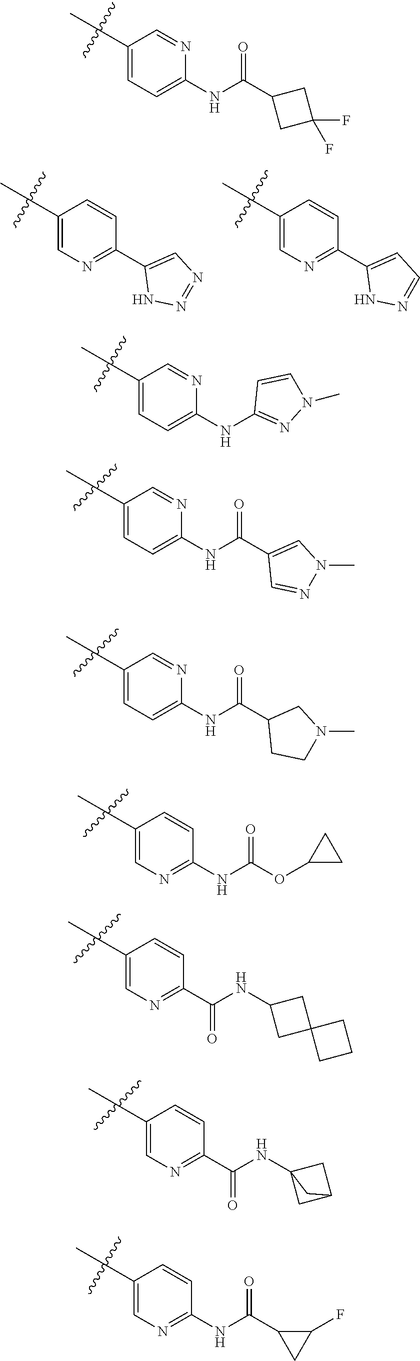

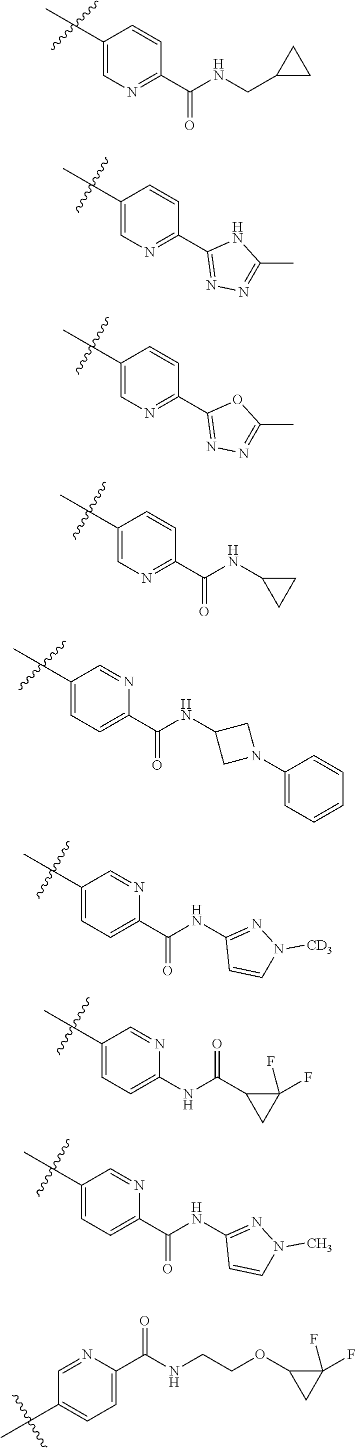

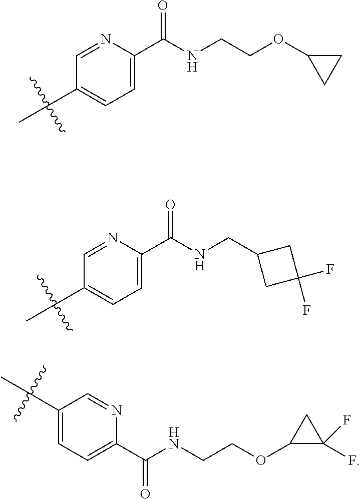 [SMILES: *c1ccc(C(=O)NCCOC2CC2)nc1 ; *c1ccc(C(=O)NCC2CC(F)(F)C2)nc1 ; *c1ccc(C(=O)NCCOC2CC2(F)F)nc1]

The tenth technical solution of the present invention relates to the compound as shown in formula (I), (II), (II-a), (II-b), (II), (III-a), (II-b), (III-c), (IV), (IV-a), (IV-b), (IV-c), (V), (VI), (VI), (VIII) or (IX), or the stereoisomer, solvate, deuterated compound, or pharmaceutically acceptable salt thereof according to the present invention, wherein ring B is selected from piperazinyl, piperidyl or

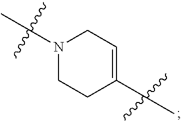 [SMILES: *C1=CCN(*)CC1]

in some embodiments, ring B is selected from piperazinyl or

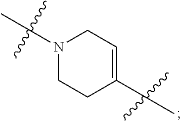 [SMILES: *C1=CCN(*)CC1]

in some embodiments, ring B is selected from piperazinyl; in some embodiments, ring B is selected from

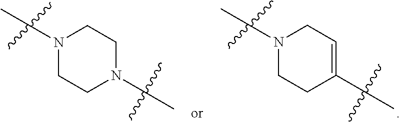 [SMILES: *N1CCN(*)CC1 ; *C1=CCN(*)CC1]

The eleventh technical solution of the present invention relates to the compound as shown in formula (I), (II), (II-a), (II-b), (III), (III-a), (III-b), (III-c), (IV), (IV-a), (IV-b), (IV-c), (V), (VI), (VII), (VIII) or (IX), or the stereoisomer, solvate, deuterated compound, or pharmaceutically acceptable salt thereof according to the present invention, wherein

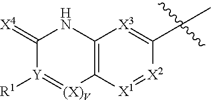

is selected from

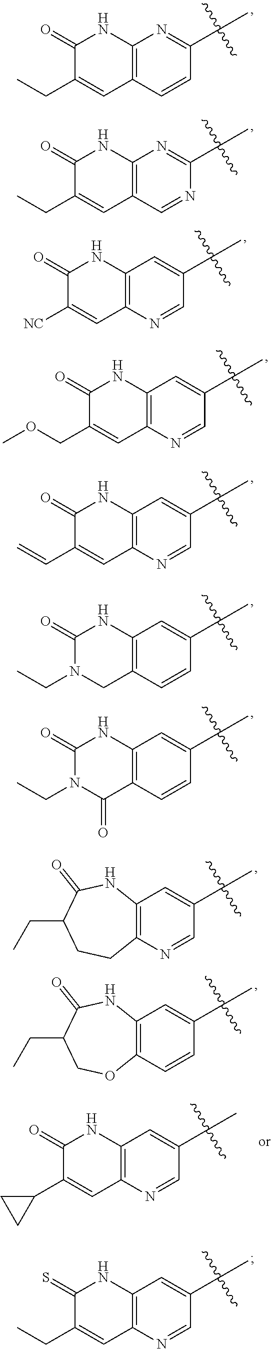

in some embodiments,

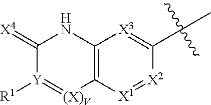

is selected from

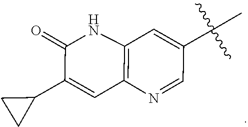 [SMILES: *c1cnc2cc(C3CC3)c(=O)[nH]c2c1]

The twelfth technical solution of the present invention relates to the compound as shown in formula (I), (II), (II-a) or (II-b), or the stereoisomer, solvate, deuterated compound, or pharmaceutically acceptable salt thereof according to the present invention, wherein

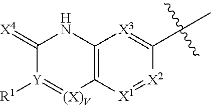

is selected from

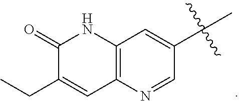 [SMILES: *c1cnc2cc(CC)c(=O)[nH]c2c1]

The thirteenth technical solution of the present invention relates to the compound as shown in formula (I), (II), (II-a), (II-b), (III), (III-a), (III-b), (III-c), (IV), (IV-a), (IV-b), (IV-c), (V), (VI), (VII), (VIII) or (IX), or the stereoisomer, solvate, deuterated compound, or pharmaceutically acceptable salt thereof, or the stereoisomer, solvate, deuterated compound, or pharmaceutically acceptable salt thereof according to the present invention, wherein

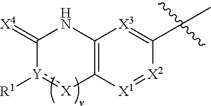

is selected from

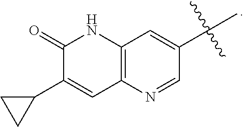 [SMILES: *c1cnc2cc(C3CC3)c(=O)[nH]c2c1]

The fourteenth technical solution of the present invention relates to the compound as shown in formula (I), or the stereoisomer, solvate, deuterated compound, or pharmaceutically acceptable salt thereof according to the present invention, having a structure of formula (V):

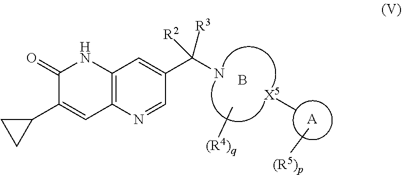

wherein R² and R³ are each independently selected from H, D, halogen, cyano, amino, hydroxyl, C₁₋₂ alkyl-O—C₁₋₂ alkyl, hydroxy C₁₋₂ alkyl, C₁₋₂ alkoxy, halo C₁₋₂ alkyl, halo C₁₋₂ alkoxy, deuterated C₁₋₂ alkyl, deuterated C₁₋₂ alkoxy or C₁₋₂ alkyl; or R² and R³ together with the carbon atom to which they are attached form C₃₋₅ cycloalkyl or 4- to 5-membered heterocycloalkyl; in some embodiments, R² and R³ are each independently selected from H, D, halogen, cyano, amino, hydroxyl, or halo C₁₋₂ alkyl; in some embodiments, R² and R³ are each independently selected from H, D, or C₁₋₂ alkyl; or R² and R³ together with the carbon atom to which they are attached form C₃₋₄ cycloalkyl; in some embodiments, R² and R³ are each independently selected from H or D;
each R⁴ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy; or two R⁴ on the same carbon atom together with the carbon atom to which they are attached form ═O; in some embodiments, each R⁴ is independently selected from D, F, Cl, cyano, amino, hydroxyl, C₁₋₂ alkyl, C₁₋₂ alkoxy, halo C₁₋₂ alkyl, halo C₁₋₂ alkoxy, deuterated C₁₋₂ alkyl, or deuterated C₁₋₂ alkoxy;
or two R⁴ on the same carbon atom together with the carbon atom to which they are attached form ═O; in some embodiments, each R⁴ is independently selected from D, F, Cl, methyl, ethyl, methoxy, ethoxy, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃; or two R⁴ on the same carbon atom together with the carbon atom to which they are attached form ═O; in some embodiments, each R⁴ is independently selected from D, F, Cl, or cyano;
each R⁵ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy; in some embodiments, each R⁵ is independently selected from D, F, Cl, C₁₋₂ alkyl, halo C₁₋₂ alkyl, or deuterated C₁₋₂ alkyl; in some embodiments, each R⁵ is independently selected from D, F, Cl, methyl, ethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃;
q is selected from 0 or 1; in some embodiments, q is selected from 0;
p is selected from 0 or 1; in some embodiments, p is selected from 0;
ring B is selected from

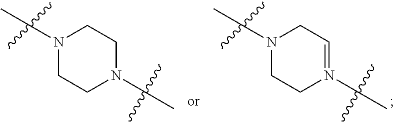

in some embodiments, ring B is selected from

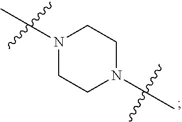 [SMILES: *N1CCN(*)CC1]

ring A is selected from

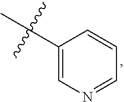 [SMILES: *c1cccnc1]

which is further substituted with 1-3 substituents selected from Rₐ;
or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b}; in some embodiments, ring A is selected from

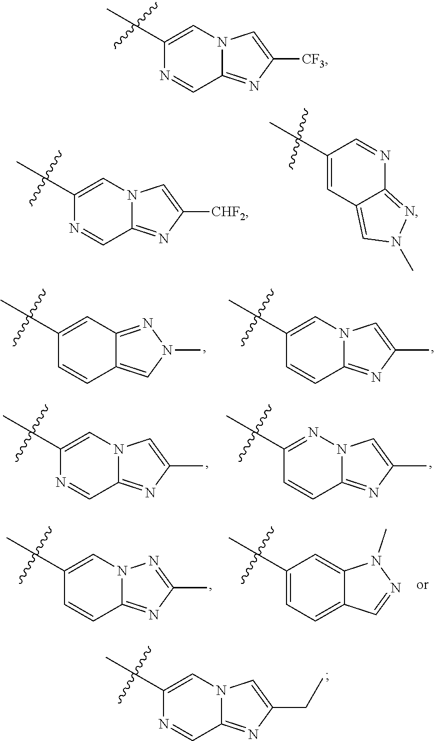

in some embodiments, ring A is selected from

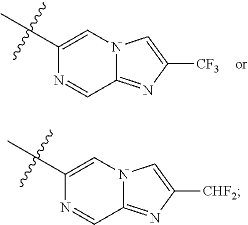 [SMILES: *c1cn2cc(C(F)(F)F)nc2cn1 ; *c1cn2cc(C(F)F)nc2cn1]

Rₐ is selected from —C(O)N(R^{a1})₂, —NR^{a1}C(O)R^{a1} or —C(O)R^{a1}; in some embodiments, Rₐ is selected from —C(O)N(R^{a1)}₂ or —NR^{a1}C(O)R^{a1}; in some embodiments, Rₐ is selected from —C(O)N(R^{a1})₂; R_{b} is selected from D, halogen, cyano, hydroxyl, amino, C₁₋₂ alkyl, C₃₋₅ cycloalkyl, 3- to 5-membered heterocycloalkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl, or deuterated C₁₋₂ alkoxy; in some embodiments, R_{b} is selected from —C(O)N(R^{a1})₂, ═O, D, halogen, cyano, hydroxyl, amino, —NHCH₃, —NHCH₂CH₃, —N(CH₃)₂, methyl, ethyl, —CH₂F, —CHF₂, —CF₃, —CH₂CH₂F, —CH₂CHF₂, —CH₂CF₃, —CHFCH₂F, —CHFCHF₂, or —CHFCF₃; in some embodiments, R_{b} is selected from D, halogen, cyano, hydroxyl, amino, methyl, ethyl, —CH₂F, —CHF₂, —CF₃, —CH₂CH₂F, —CH₂CHF₂, —CH₂CF₃, —CHFCH₂F, —CHFCHF₂, or —CHFCF₃; each R^{a1} is independently selected from H, D, C₁₋₂ alkyl, C₃₋₅ cycloalkyl, 3- to 5-membered heterocycloalkyl, 5- to 6-membered heteroaryl, halo C₁₋₂ alkyl, halo C₁₋₂ alkoxy, deuterated C₁₋₂ alkyl, or deuterated C₁₋₂ alkoxy, wherein the cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, or C₃₋₄ cycloalkyl; in some embodiments, each R^{a1} is independently selected from C₁₋₂ alkyl, C₃₋₅ cycloalkyl, 5- to 6-membered heteroaryl, halo C₁₋₂ alkyl, halo C₁₋₂ alkoxy, deuterated C₁₋₂ alkyl, or deuterated C₁₋₂ alkoxy, wherein the cycloalkyl or heteroaryl is optionally further substituted with 1-3 substituents selected from D, halogen, cyano, hydroxyl, amino, C₁₋₂ alkyl, halo C₁₋₂ alkyl, or deuterated C₁₋₂ alkyl; in some embodiments, each R^{a1} is independently selected from C₁₋₂ alkyl, C₃₋₅ cycloalkyl, or 5- to 6-membered heteroaryl, wherein the cycloalkyl or heteroaryl is optionally further substituted with 1-3 substituents selected from D, halogen, cyano, hydroxyl, amino, C₁₋₂ alkyl, halo C₁₋₂ alkyl, or deuterated C₁₋₂ alkyl; in some embodiments, each R^{a1} is independently selected from methyl, ethyl, cyclopropyl, cyclobutyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, —CH₂CH₂CF₃, or

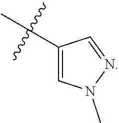 [SMILES: *c1cnn(C)c1]

The fifteenth technical solution of the present invention relates to the compound as shown in formula (I), or the stereoisomer, solvate, deuterated compound, or pharmaceutically acceptable salt thereof according to the present invention, having a structure of formula (VI), (VII), (VIII) or (IX):

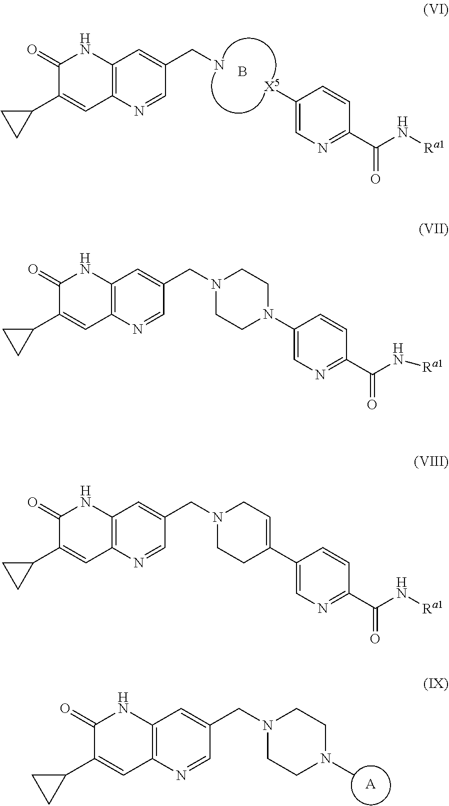

wherein ring B is selected from

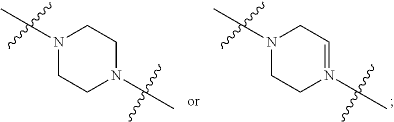

in some embodiments, ring B is selected from

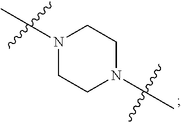 [SMILES: *N1CCN(*)CC1]

X⁵ is selected from N or CH; in some embodiments, X⁵ is selected from N;
each R^{a1} is independently selected from C₁₋₄ alkyl, C₃₋₅ cycloalkyl, or 5- to 6-membered heteroaryl, wherein the alkyl, cycloalkyl, or heteroaryl is optionally further substituted with 1, 2 or 3 substituents selected from D, F, Cl, cyano, hydroxyl, amino, C₁₋₂ alkyl, halo C₁₋₂ alkyl, or deuterated C₁₋₂ alkyl; in some embodiments, each R^{a1} is independently selected from methyl, ethyl, propyl, isopropyl, tert-butyl, cyclopropyl, cyclobutyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, —CH₂CH₂CF₃,

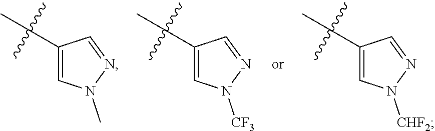

ring A is selected from 8- to 10-membered bicyclic heteroaromatic ring containing 1-5 nitrogen, oxygen or sulfur atoms, wherein the heteroaromatic ring is optionally further substituted with 1, 2 or 3 substituents selected from —C(O)N(R^{a1})₂, —NHC₁₋₂ alkyl, —N(C₁₋₂ alkyl)₂, C₁₋₂ alkyl, ═O, C₁₋₂ alkyl, C₃₋₅ cycloalkyl, halo C₁₋₂ alkyl, halo C₁₋₂ alkoxy, deuterated C₁₋₂ alkyl, or deuterated C₁₋₂ alkoxy; in some embodiments, ring A is selected from

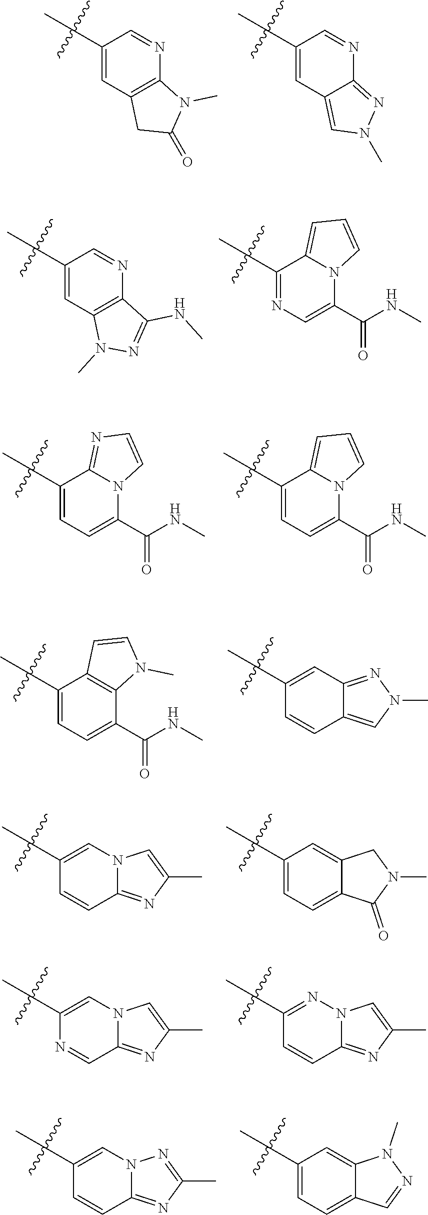

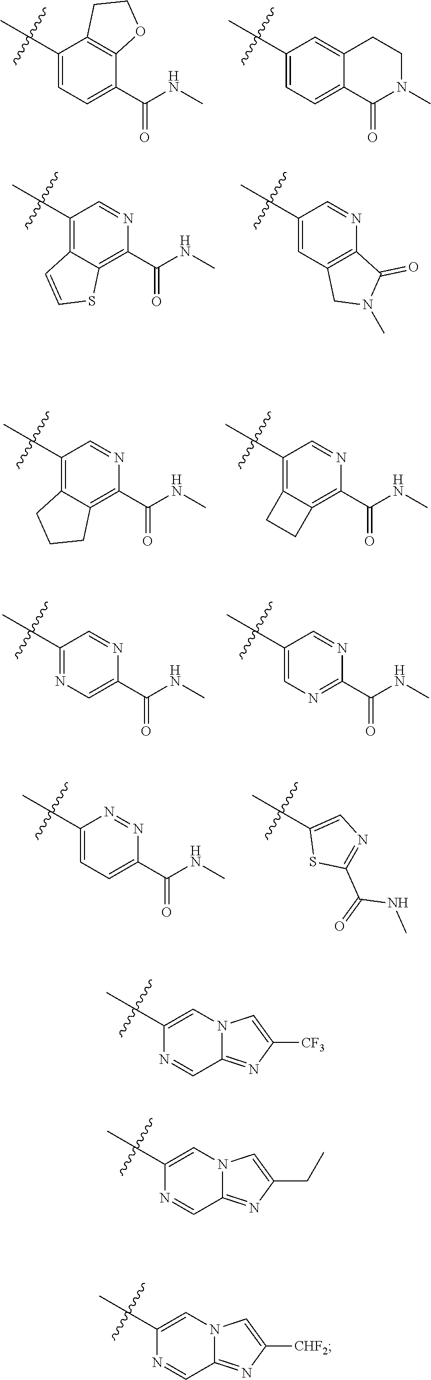

in some embodiments, ring A is selected from 8- to 10-membered bicyclic heteroaromatic ring containing 1-5 nitrogen, oxygen or sulfur atoms, wherein the heteroaromatic ring is optionally further substituted with 1, 2 or 3 substituents selected from C₁₋₂ alkyl, C₃₋₅ cycloalkyl, halo C₁₋₂ alkyl, halo C₁₋₂ alkoxy, deuterated C₁₋₂ alkyl, or deuterated C₁₋₂ alkoxy; in some embodiments, ring A is selected from 8-, 9-or 10-membered bicyclic heteroaromatic ring containing 1, 2, 3, 4 or 5 nitrogen, oxygen or sulfur atoms, wherein the heteroaromatic ring is optionally further substituted with 1, 2 or 3 substituents selected from C₁₋₂ alkyl, C₃₋₅ cycloalkyl, halo C₁₋₂ alkyl, halo C₁₋₂ alkoxy, deuterated C₁₋₂ alkyl, or deuterated C₁₋₂ alkoxy; in some embodiments, ring A is selected from

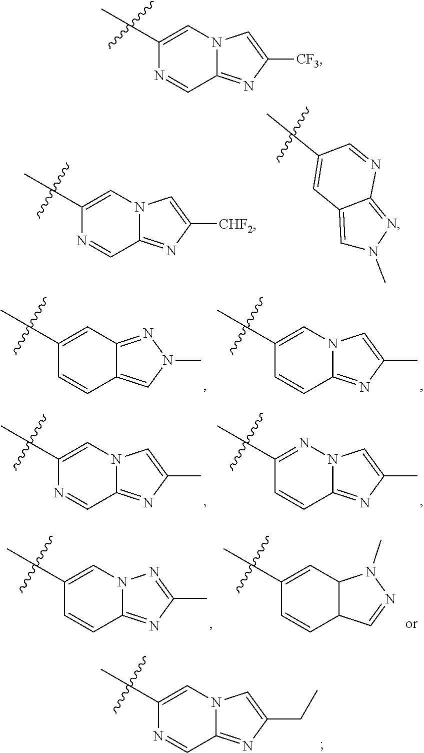

in some embodiments, ring A is selected from

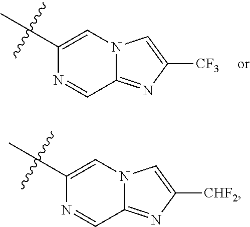 [SMILES: *c1cn2cc(C(F)(F)F)nc2cn1 ; *c1cn2cc(C(F)F)nc2cn1]

The sixteenth technical solution of the present invention relates to the compound as shown in formula (I), (II), (II-a), (II-b), (III), (III-a), (III-b), (III-c), (IV), (IV-a), (IV-b), (IV-c), (V), (VI), (VII), (VIII) or (IX), or the stereoisomer, solvate, deuterated compound, or pharmaceutically acceptable salt thereof according to the present invention, wherein the compound is selected from one of the following structures:

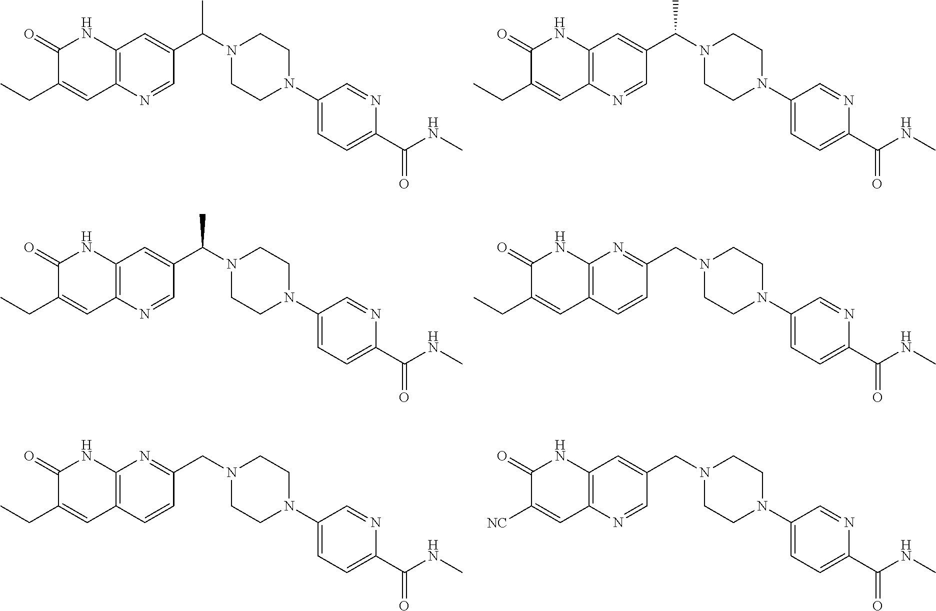 [SMILES: CCc1cc2ncc(C(C)N3CCN(c4ccc(C(=O)NC)nc4)CC3)cc2[nH]c1=O ; CCc1cc2ncc([C@H](C)N3CCN(c4ccc(C(=O)NC)nc4)CC3)cc2[nH]c1=O ; CCc1cc2ncc([C@@H](C)N3CCN(c4ccc(C(=O)NC)nc4)CC3)cc2[nH]c1=O ; CCc1cc2ccc(CN3CCN(c4ccc(C(=O)NC)nc4)CC3)nc2[nH]c1=O ; CCc1cc2ccc(CN3CCN(c4ccc(C(=O)NC)nc4)CC3)nc2[nH]c1=O ; CNC(=O)c1ccc(N2CCN(Cc3cnc4cc(C#N)c(=O)[nH]c4c3)CC2)cn1]

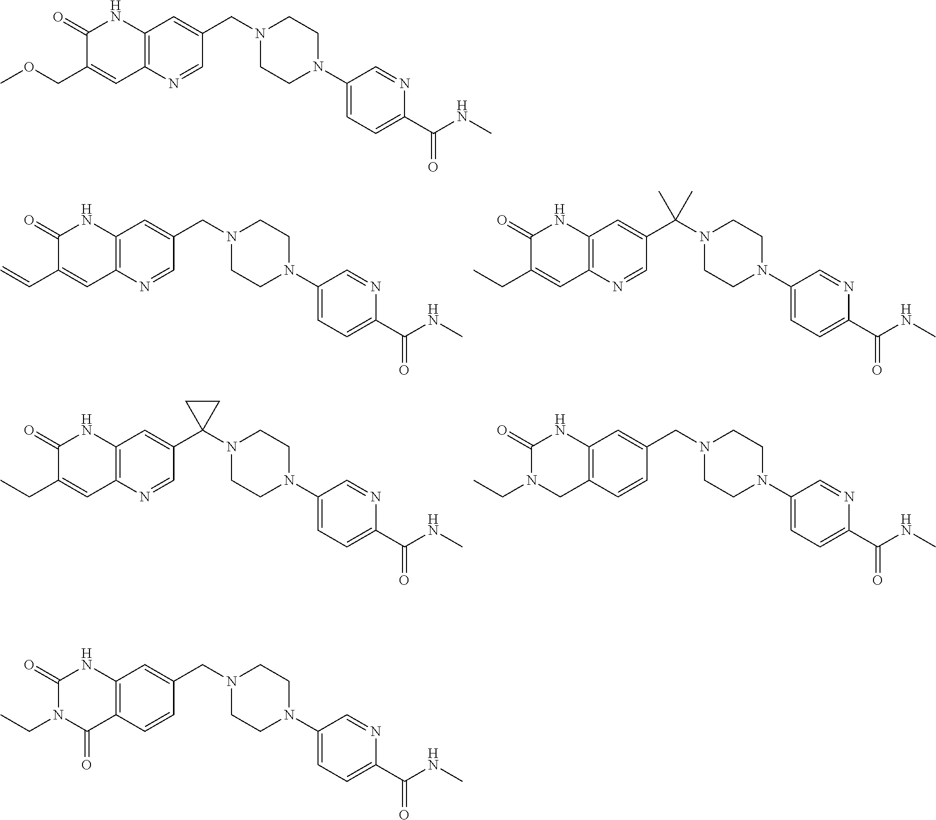

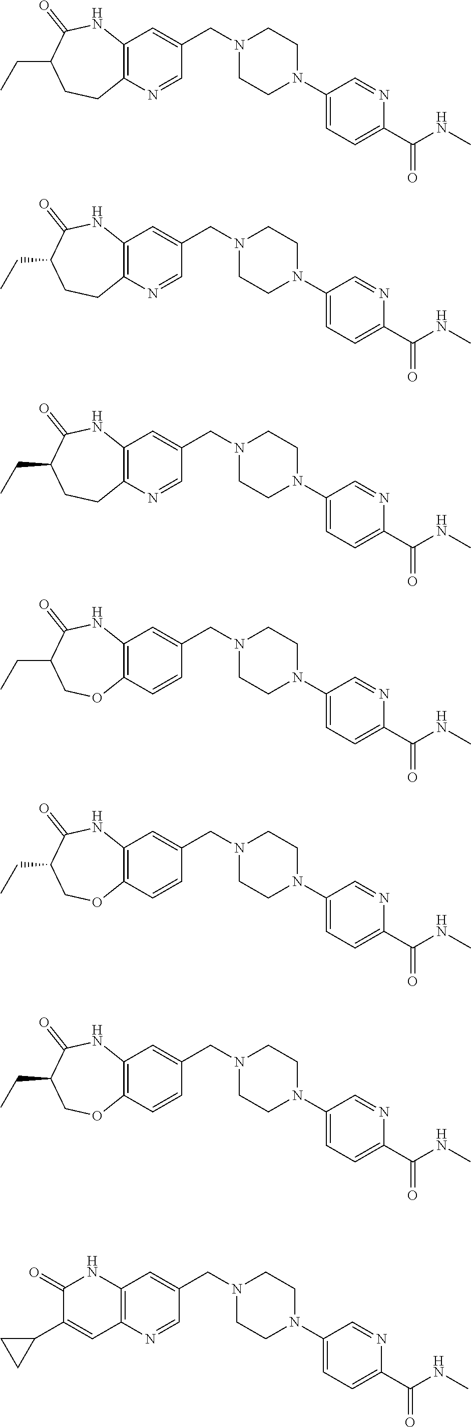

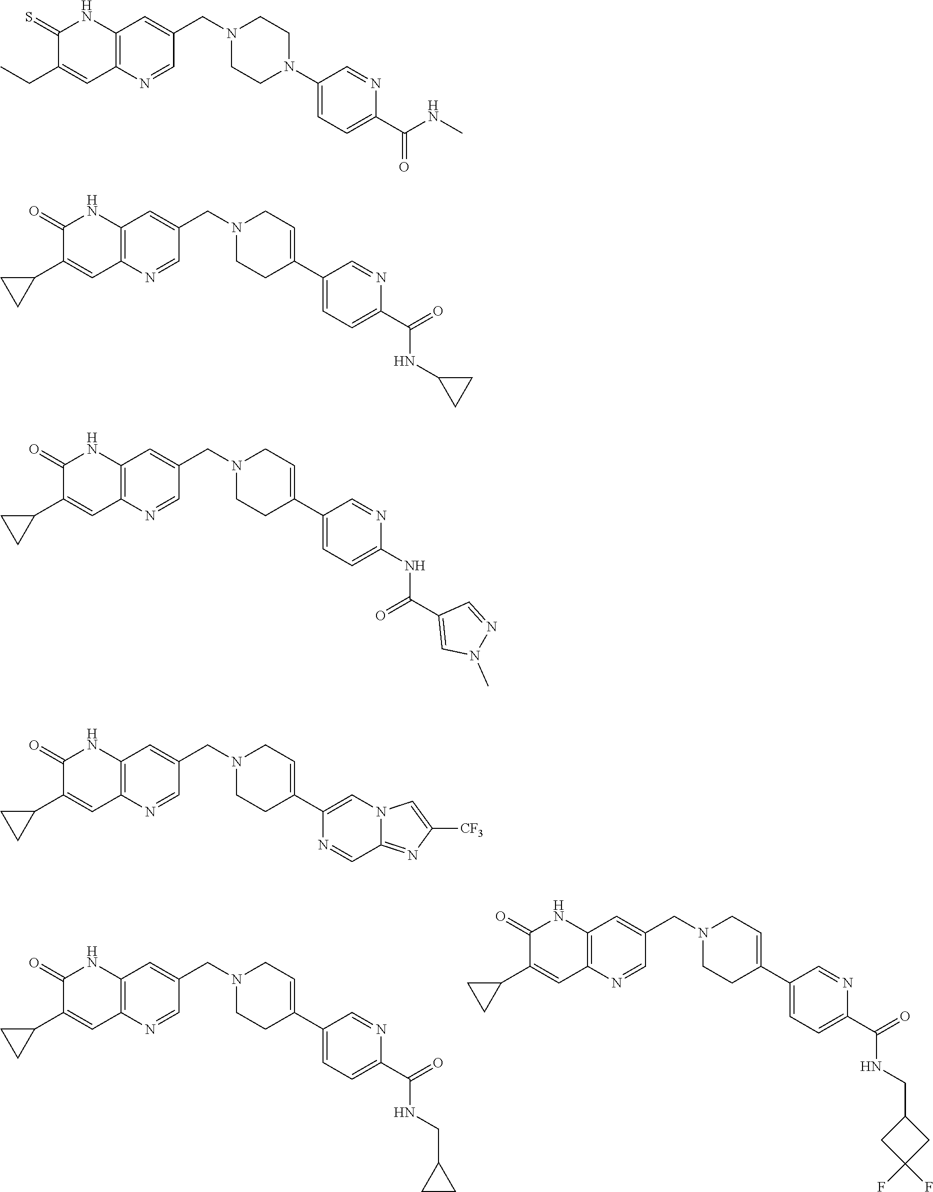 [SMILES: CCc1cc2ncc(CN3CCN(c4ccc(C(=O)NC)nc4)CC3)cc2[nH]c1=S ; O=C(NC1CC1)c1ccc(C2=CCN(Cc3cnc4cc(C5CC5)c(=O)[nH]c4c3)CC2)cn1 ; Cn1cc(C(=O)Nc2ccc(C3=CCN(Cc4cnc5cc(C6CC6)c(=O)[nH]c5c4)CC3)cn2)cn1 ; O=c1[nH]c2cc(CN3CC=C(c4cn5cc(C(F)(F)F)nc5cn4)CC3)cnc2cc1C1CC1 ; O=C(NCC1CC1)c1ccc(C2=CCN(Cc3cnc4cc(C5CC5)c(=O)[nH]c4c3)CC2)cn1 ; O=C(NCC1CC(F)(F)C1)c1ccc(C2=CCN(Cc3cnc4cc(C5CC5)c(=O)[nH]c4c3)CC2)cn1]

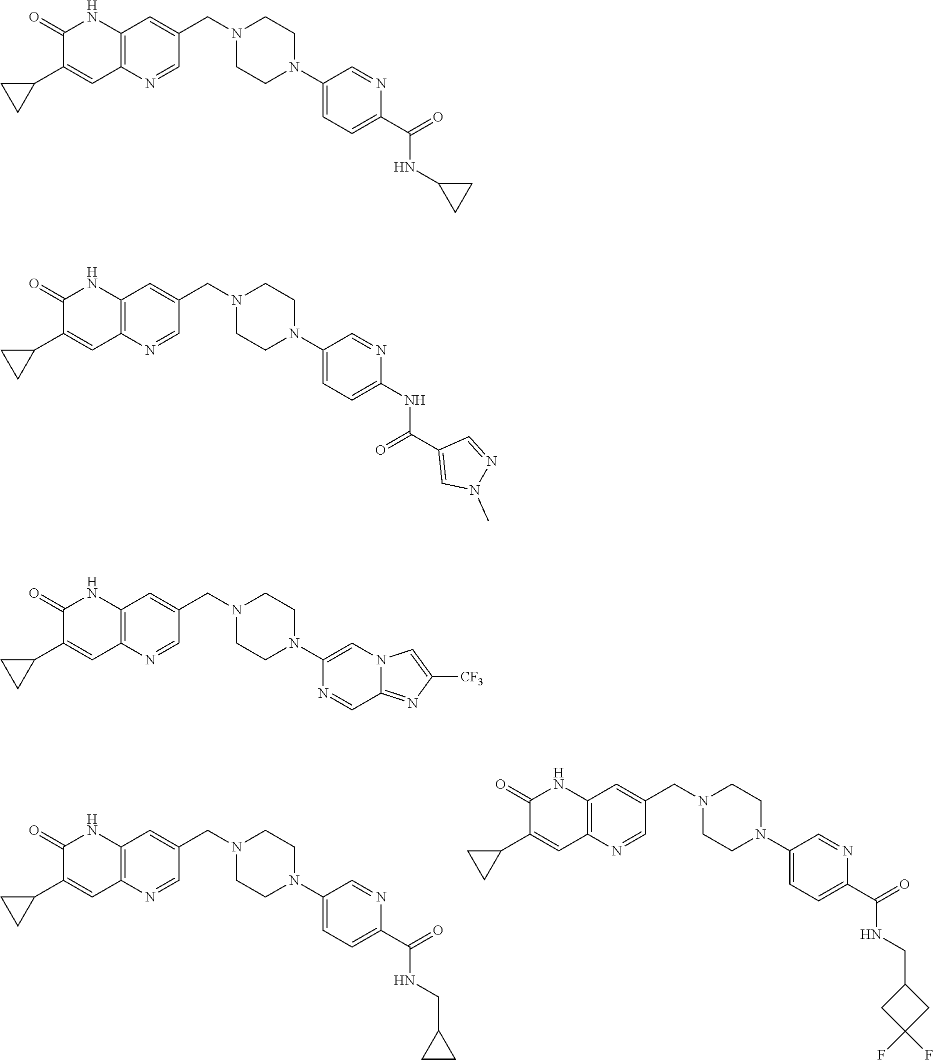 [SMILES: O=C(NC1CC1)c1ccc(N2CCN(Cc3cnc4cc(C5CC5)c(=O)[nH]c4c3)CC2)cn1 ; Cn1cc(C(=O)Nc2ccc(N3CCN(Cc4cnc5cc(C6CC6)c(=O)[nH]c5c4)CC3)cn2)cn1 ; O=c1[nH]c2cc(CN3CCN(c4cn5cc(C(F)(F)F)nc5cn4)CC3)cnc2cc1C1CC1 ; O=C(NCC1CC1)c1ccc(N2CCN(Cc3cnc4cc(C5CC5)c(=O)[nH]c4c3)CC2)cn1 ; O=C(NCC1CC(F)(F)C1)c1ccc(N2CCN(Cc3cnc4cc(C5CC5)c(=O)[nH]c4c3)CC2)cn1]

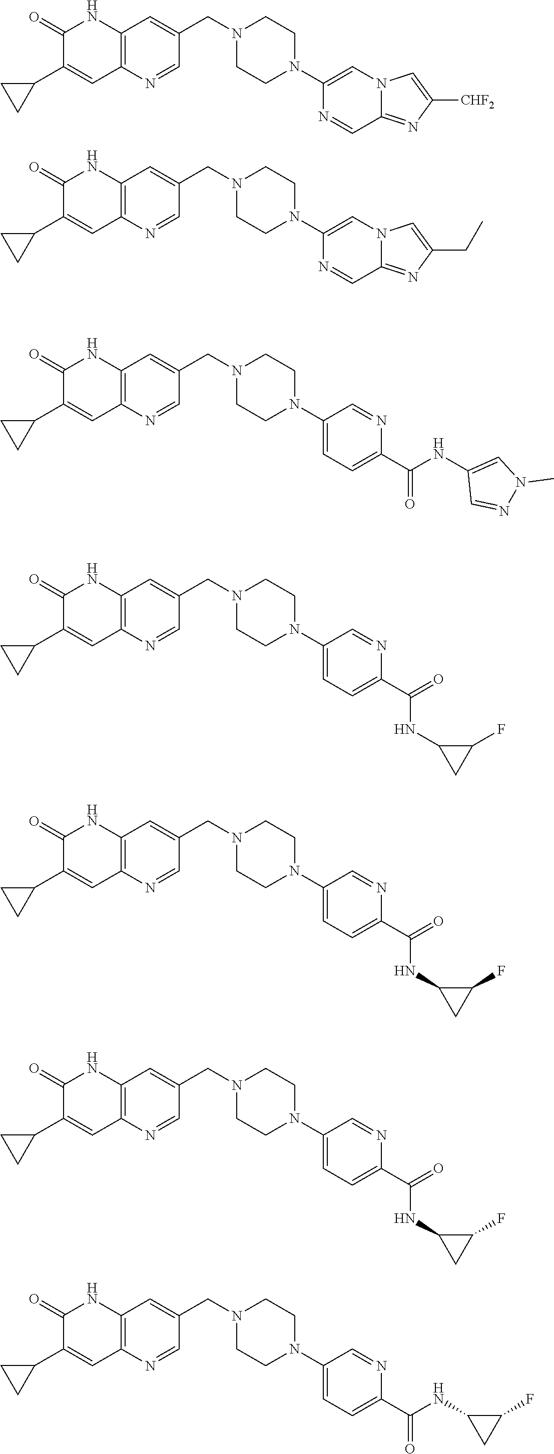

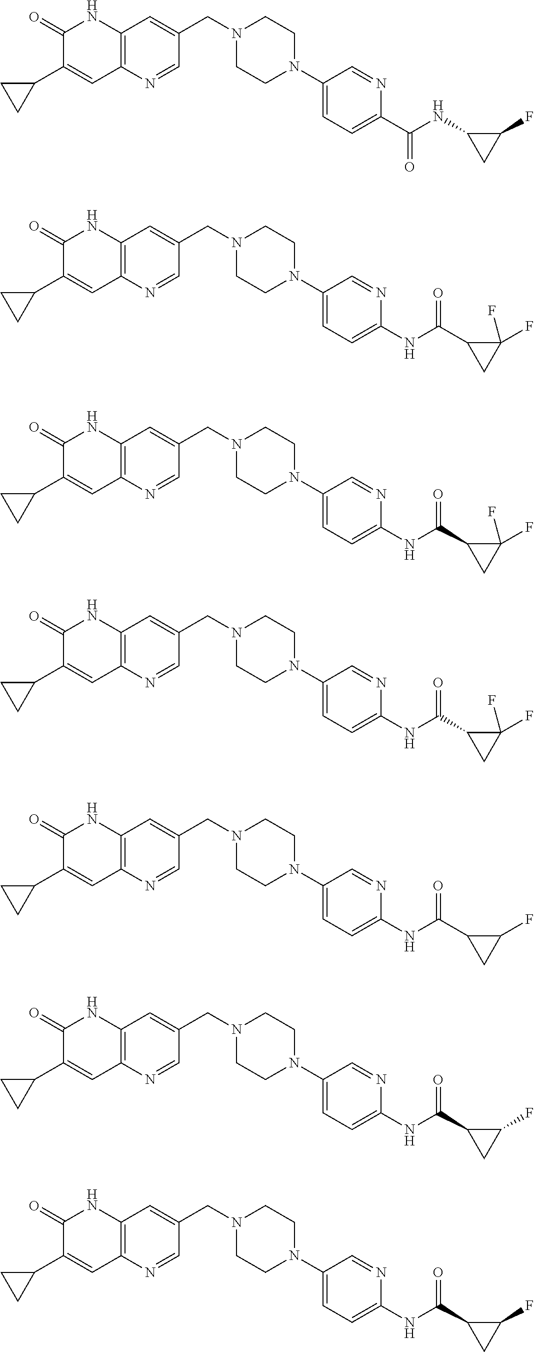

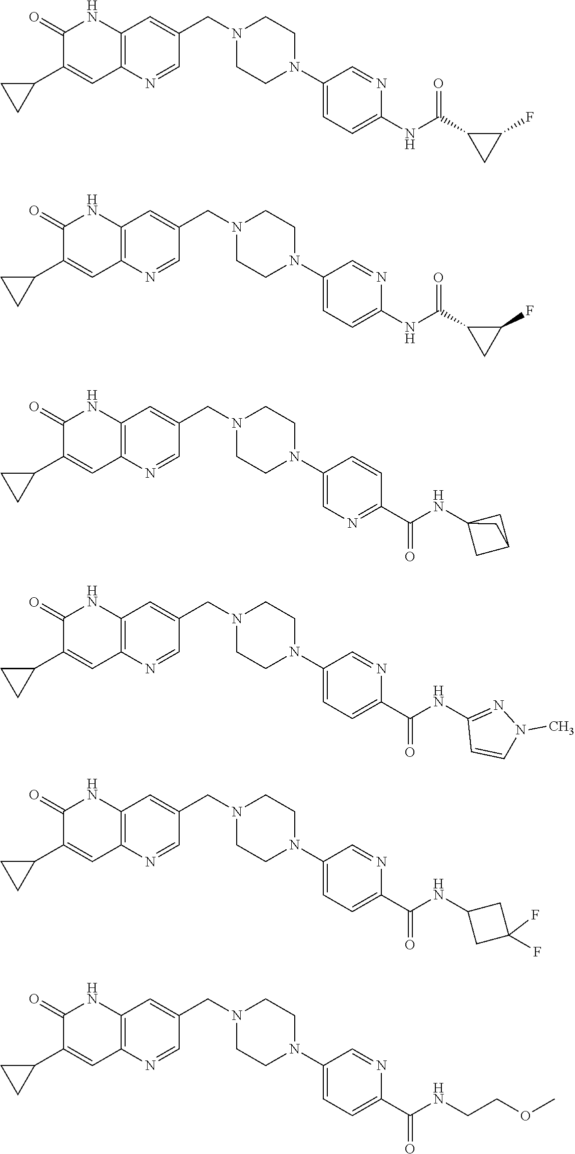 [SMILES: O=C(Nc1ccc(N2CCN(Cc3cnc4cc(C5CC5)c(=O)[nH]c4c3)CC2)cn1)[C@H]1C[C@H]1F ; O=C(Nc1ccc(N2CCN(Cc3cnc4cc(C5CC5)c(=O)[nH]c4c3)CC2)cn1)[C@H]1C[C@@H]1F ; O=C(NC12CC(C1)C2)c1ccc(N2CCN(Cc3cnc4cc(C5CC5)c(=O)[nH]c4c3)CC2)cn1 ; Cn1ccc(NC(=O)c2ccc(N3CCN(Cc4cnc5cc(C6CC6)c(=O)[nH]c5c4)CC3)cn2)n1 ; O=C(NC1CC(F)(F)C1)c1ccc(N2CCN(Cc3cnc4cc(C5CC5)c(=O)[nH]c4c3)CC2)cn1 ; COCCNC(=O)c1ccc(N2CCN(Cc3cnc4cc(C5CC5)c(=O)[nH]c4c3)CC2)cn1]

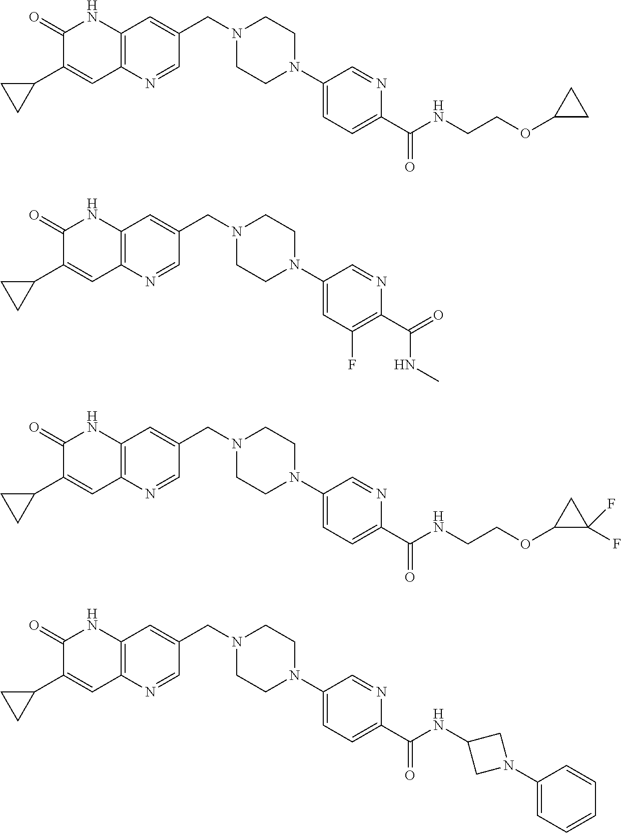 [SMILES: O=C(NCCOC1CC1)c1ccc(N2CCN(Cc3cnc4cc(C5CC5)c(=O)[nH]c4c3)CC2)cn1 ; CNC(=O)c1ncc(N2CCN(Cc3cnc4cc(C5CC5)c(=O)[nH]c4c3)CC2)cc1F ; O=C(NCCOC1CC1(F)F)c1ccc(N2CCN(Cc3cnc4cc(C5CC5)c(=O)[nH]c4c3)CC2)cn1 ; O=C(NC1CN(c2ccccc2)C1)c1ccc(N2CCN(Cc3cnc4cc(C5CC5)c(=O)[nH]c4c3)CC2)cn1]

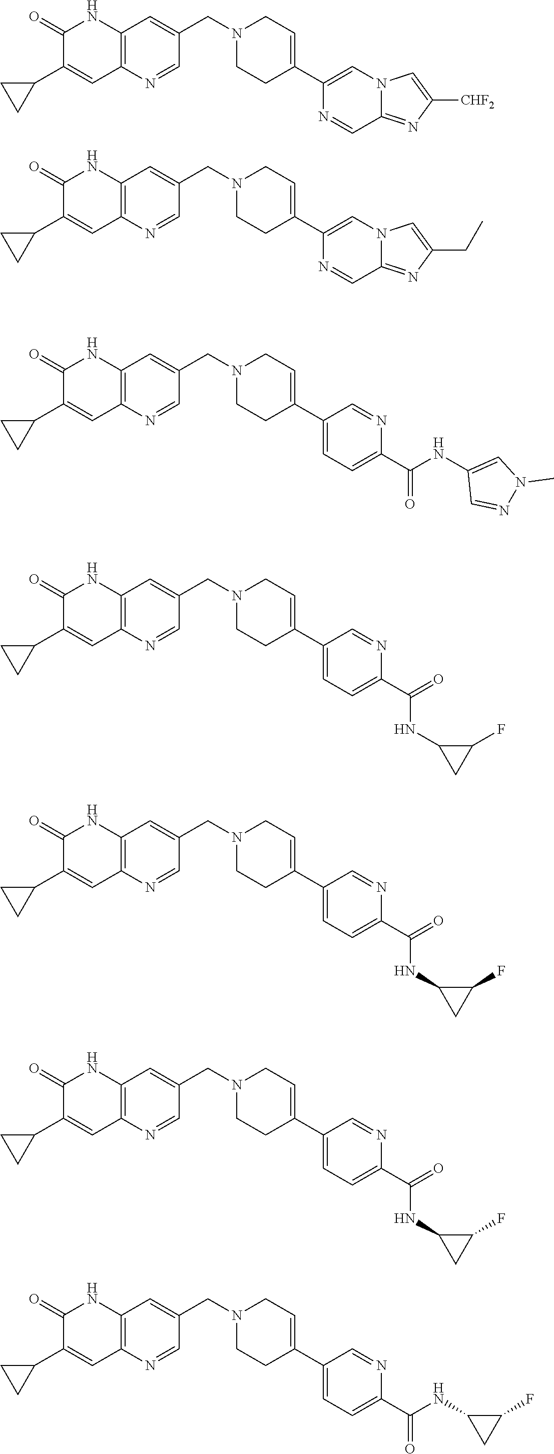

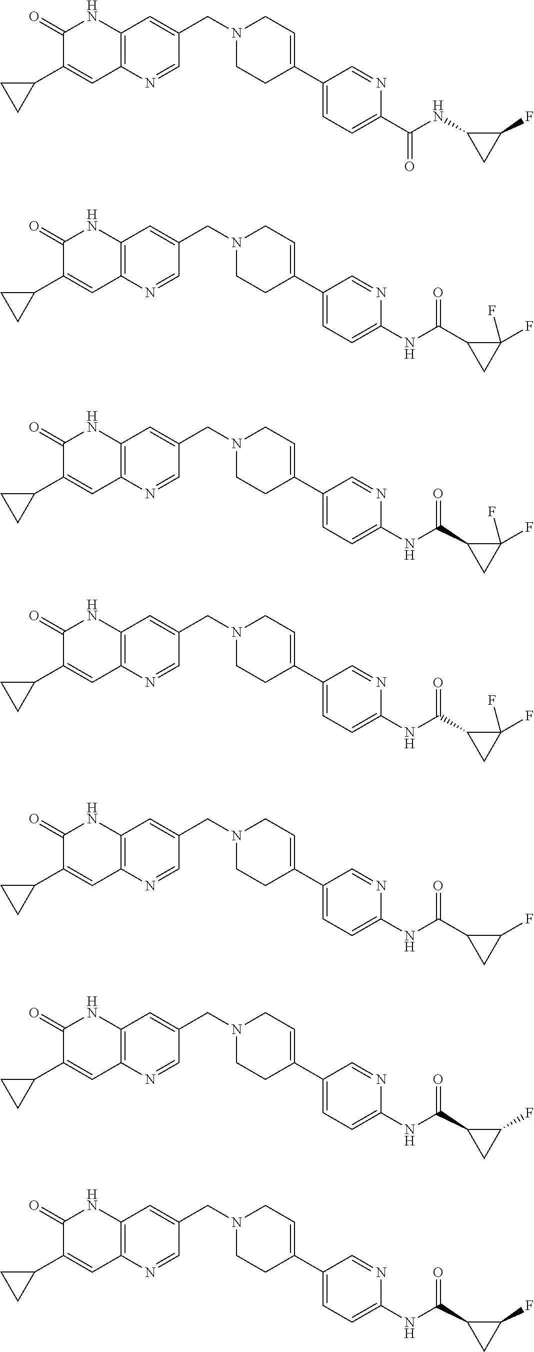

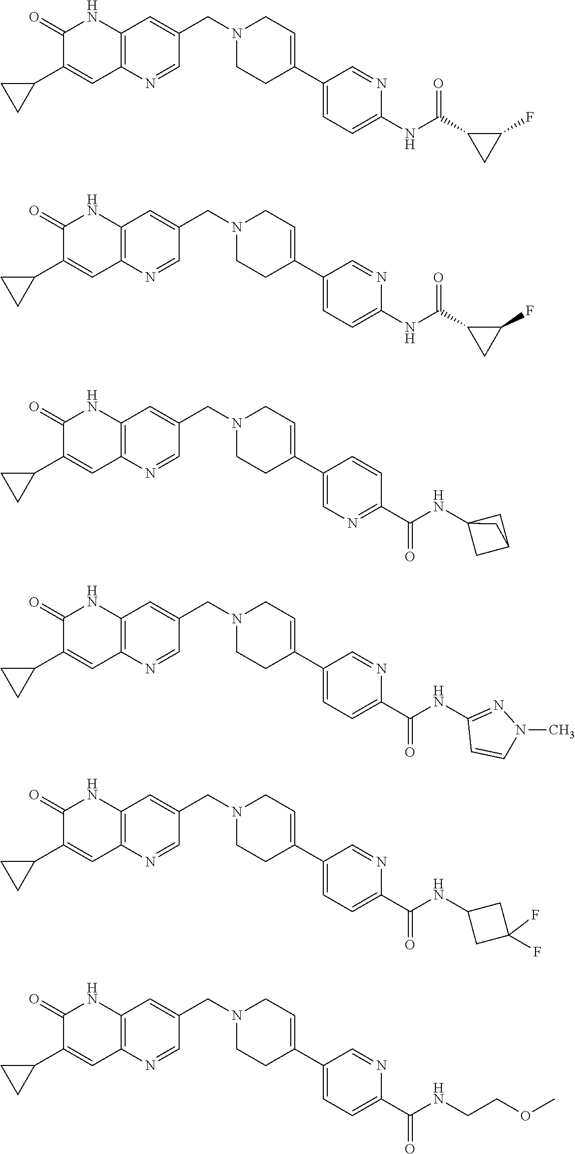 [SMILES: O=C(Nc1ccc(C2=CCN(Cc3cnc4cc(C5CC5)c(=O)[nH]c4c3)CC2)cn1)[C@H]1C[C@H]1F ; O=C(Nc1ccc(C2=CCN(Cc3cnc4cc(C5CC5)c(=O)[nH]c4c3)CC2)cn1)[C@H]1C[C@@H]1F ; O=C(NC12CC(C1)C2)c1ccc(C2=CCN(Cc3cnc4cc(C5CC5)c(=O)[nH]c4c3)CC2)cn1 ; Cn1ccc(NC(=O)c2ccc(C3=CCN(Cc4cnc5cc(C6CC6)c(=O)[nH]c5c4)CC3)cn2)n1 ; O=C(NC1CC(F)(F)C1)c1ccc(C2=CCN(Cc3cnc4cc(C5CC5)c(=O)[nH]c4c3)CC2)cn1 ; COCCNC(=O)c1ccc(C2=CCN(Cc3cnc4cc(C5CC5)c(=O)[nH]c4c3)CC2)cn1]

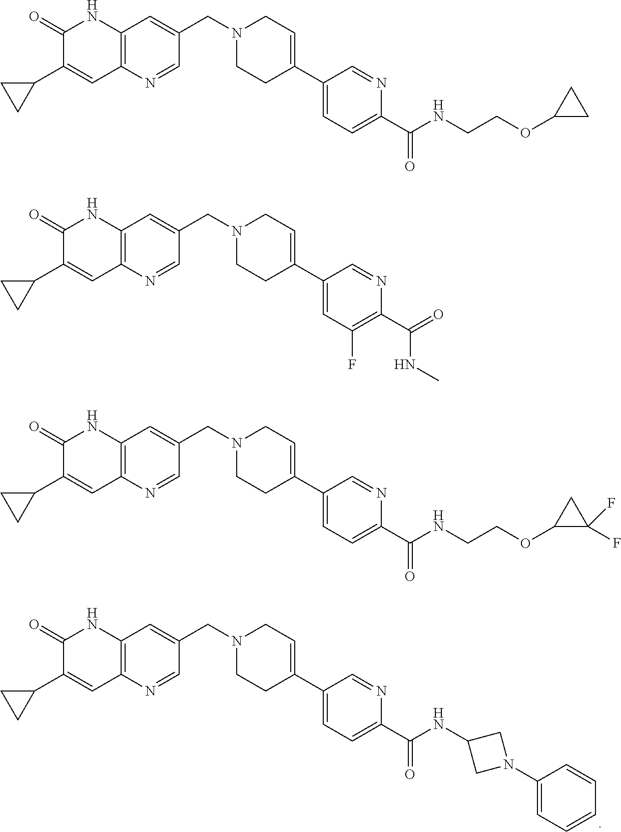 [SMILES: O=C(NCCOC1CC1)c1ccc(C2=CCN(Cc3cnc4cc(C5CC5)c(=O)[nH]c4c3)CC2)cn1 ; CNC(=O)c1ncc(C2=CCN(Cc3cnc4cc(C5CC5)c(=O)[nH]c4c3)CC2)cc1F ; O=C(NCCOC1CC1(F)F)c1ccc(C2=CCN(Cc3cnc4cc(C5CC5)c(=O)[nH]c4c3)CC2)cn1 ; O=C(NC1CN(c2ccccc2)C1)c1ccc(C2=CCN(Cc3cnc4cc(C5CC5)c(=O)[nH]c4c3)CC2)cn1]

Secondly, the present invention also provides a pharmaceutical composition, comprising the compound, or the stereoisomer, solvate, deuterated compound, or pharmaceutically acceptable salt thereof according to any one of the preceding technical solutions, and a pharmaceutically acceptable excipient and/or carrier.

Further, the pharmaceutical composition or pharmaceutical preparation comprises 1-1440 mg of the compound, or the stereoisomer, deuterated compound, solvate, or pharmaceutically acceptable salt thereof according to any one of the preceding first to fourteenth technical solutions, and a pharmaceutically acceptable excipient and/or carrier.

Further, the present invention also provides the use of the compound, or the stereoisomer, solvate, deuterated compound, or pharmaceutically acceptable salt thereof, or the pharmaceutical composition according to any one of the preceding embodiments in the preparation of a drug for treating/preventing a PARP-1-mediated disease. Further, the PARP-1-mediated disease includes, but is not limited to cancer.

The present invention also provides a method for treating a disease in a mammal, comprising administering to a subject a therapeutically effective amount of the compound, or the stereoisomer, deuterated compound, solvate, or pharmaceutically acceptable salt thereof according to any one of the preceding first to fourteenth technical solutions, and a pharmaceutically acceptable excipient and/or carrier, wherein the therapeutically effective amount is preferably 1-1440 mg, and the disease is preferably cancer.

The present invention also provides a method for treating a disease in a mammal, comprising administering to the mammal a therapeutically effective amount of the compound, or the stereoisomer, deuterated compound, solvate, or pharmaceutically acceptable salt thereof, or the pharmaceutical composition according to the present invention. In some embodiments, the mammal according to the present invention comprises humans.

The “effective amount” or “therapeutically effective amount” as described in the present application refers to administration of a sufficient amount of the compound disclosed in the present application that will alleviate to some extent one or more symptoms of the diseases or conditions being treated. In some embodiments, the outcome is the reduction and/or remission of signs, symptoms or causes of the disease, or any other desired change in the biological system. For example, an “effective amount” in terms of the therapeutic use is an amount of the composition comprising the compound disclosed in the present application that is required to provide clinically significant reduction of the symptoms of the disease. Examples of the therapeutically effective amount include, but are not limited to 1-1440 mg, 1-1400 mg, 1-1300 mg, 1-1200 mg, 1-1000 mg, 1-900 mg, 1-800 mg, 1-700 mg, 1-600 mg, 1-500 mg, 1-400 mg, 1-300 mg, 1-250 mg, 1-200 mg, 1-150 mg, 1-125 mg, 1-100 mg, 1-80 mg, 1-60 mg, 1-50 mg, 1-40 mg, 1-25 mg, 1-20 mg, 5-1000 mg, 5-900 mg, 5-800 mg, 5-700 mg, 5-600 mg, 5-500 mg, 5-400 mg, 5-300 mg, 5-250 mg, 5-200 mg, 5-150 mg, 5-125 mg, 5-100 mg, 5-90 mg, 5-70 mg, 5-80 mg, 5-60 mg, 5-50 mg, 5-40 mg, 5-30 mg, 5-25 mg, 5-20 mg, 10-1000 mg, 10-900 mg, 10-800 mg, 10-700 mg, 10-600 mg, 10-500 mg, 10-450 mg, 10-400 mg, 10-300 mg, 10-250 mg, 10-200 mg, 10-150 mg, 10-125 mg, 10-100 mg, 10-90 mg, 10-80 mg, 10-70 mg, 10-60 mg, 10-50 mg, 10-40 mg, 10-30 mg, 10-20 mg; 20-1000 mg, 20-900 mg, 20-800 mg, 20-700 mg, 20-600 mg, 20-500 mg, 20-400 mg, 20-350 mg, 20-300 mg, 20-250 mg, 20-200 mg, 20-150 mg, 20-125 mg, 20-100 mg, 20-90 mg, 20-80 mg, 20-70 mg, 20-60 mg, 20-50 mg, 20-40 mg, 20-30 mg; 50-1000 mg, 50-900 mg, 50-800 mg, 50-700 mg, 50-600 mg, 50-500 mg, 50-400 mg, 50-300 mg, 50-250 mg, 50-200 mg, 50-150 mg, 50-125 mg, 50-100 mg; 100-1000 mg, 100-900 mg, 100-800 mg, 100-700 mg, 100-600 mg, 100-500 mg, 100-400 mg, 100-300 mg, 100-250 mg, or 100-200 mg.

The present invention relates to a pharmaceutical composition or pharmaceutical preparation, comprising a therapeutically effective amount of the compound, or the stereoisomer, deuterated compound, solvate, or pharmaceutically acceptable salt thereof according to the present invention, and an excipient and/or carrier. The pharmaceutical composition can be in a unit preparation form (the amount of the drug substance in the unit preparation is also referred to as the “preparation strength”). In some embodiments, the pharmaceutical composition comprises the compound, or the stereoisomer, deuterated compound, solvate, or pharmaceutically acceptable salt thereof according to the present invention in an amount including but not limited to 1-1440 mg, 5-1000 mg, 10-800 mg, 20-600 mg, 25-500 mg, 40-200 mg, 50-100 mg, 1 mg, 1.25 mg, 2.5 mg, 5 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 900 mg, 950 mg, 1000 mg, 1100 mg, 1200 mg, 1300 mg, 1400 mg, or 1440 mg.

The present invention also provides a method for treating a disease in a mammal, comprising administering to a subject a therapeutically effective amount of the compound, or the stereoisomer, deuterated compound, solvate, or pharmaceutically acceptable salt thereof according to the present invention, and a pharmaceutically acceptable excipient and/or carrier, wherein the therapeutically effective amount is preferably 1-1440 mg, and the disease is preferably cancer.

The present invention also provides a method for treating a disease in a mammal, comprising administering to a subject a drug, i.e., the compound, or the stereoisomer, deuterated material, solvate, or pharmaceutically acceptable salt thereof according to the present invention, and a pharmaceutically acceptable excipient and/or carrier in a daily dose of 1-1440 mg/day, wherein the daily dose can be a single dose or divided doses; in some embodiments, the daily dose includes, but is not limited to 10-1440 mg/day, 20-1440 mg/day, 25-1440 mg/day, 50-1440 mg/day, 75-1440 mg/day, 100-1440 mg/day, 200-1440 mg/day, 10-1000 mg/day, 20-1000 mg/day, 25-1000 mg/day, 50-1000 mg/day, 75-1000 mg/day, 100-1000 mg/day, 200-1000 mg/day, 25-800 mg/day, 50-800 mg/day, 100-800 mg/day, 200-800 mg/day, 25-400 mg/day, 50-400 mg/day, 100-400 mg/day, or 200-400 mg/day; in some embodiments, the daily dose includes, but is not limited to 1 mg/day, 5 mg/day, 10 mg/day, 20 mg/day, 25 mg/day, 50 mg/day, 75 mg/day, 100 mg/day, 125 mg/day, 150 mg/day, 200 mg/day, 400 mg/day, 600 mg/day, 800 mg/day, 1000 mg/day, 1200 mg/day, 1400 mg/day, or 1440 mg/day.

The present invention relates to a kit, which may include a composition in single-dose or multiple-dose form, wherein the kit comprises the compound, or the stereoisomer, deuterated compound, solvate, or pharmaceutically acceptable salt thereof according to the present invention, and the amount of the compound, or the stereoisomer, deuterated compound, solvate, or pharmaceutically acceptable salt thereof according to the present invention is the same as that in the above-mentioned pharmaceutical composition.

In the present invention, the amount of the compound, or the stereoisomer, deuterated compound, solvate, or pharmaceutically acceptable salt thereof according to the present invention is calculated in the form of a free base in each case.

The term “preparation strength” refers to the weight of the drug substance contained in each vial, tablet or other unit preparation.

### Synthetic Route

Patent document WO 2021013735 A1 introduces a method for preparing a PARP-1 inhibitor, and those skilled in the art would have been able to prepare the compounds of the present invention by means of combining the document and known organic synthesis techniques, wherein the starting materials used therein are commercially available chemicals and (or) compounds described in chemical documents. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific Co., Ltd., etc.

References and monographs in the art introduce in detail the synthesis of reactants that can be used to prepare the compounds described herein, or provide articles describing the preparation method for reference. The references and monographs include: “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992; Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups”, John Wiley & Sons, in 73 volumes.

Specific and similar reactants can be selectively identified by the indexes of known chemicals prepared by the Chemical Abstracts Service of the American Chemical Society, wherein the indexes are available in most public libraries or university libraries and online. Chemicals that are known but not commercially available in the catalog are optionally prepared by custom chemical synthesis plants, wherein many of standard chemical supply plants (such as those listed above) provide custom synthesis services. Reference document for the preparation and selection of the pharmaceutically acceptable salts of the compounds described herein is P. H. Stahl & C. G. Wermuth “Handbook of Pharmaceutical Salts”, Verlag Helvetica Chimica Acta, Zurich, 2002.

### Term

Unless otherwise specified, the terms of the present invention have the following meanings.

The carbon, hydrogen, oxygen, sulfur, nitrogen and halogen involved in the groups and compounds of the present invention all comprise isotopes thereof, and are optionally further substituted with one or more of the corresponding isotopes thereof, wherein the isotopes of carbon comprise ¹²C, ¹³C and ¹⁴C; the isotopes of hydrogen comprise protium (H), deuterium (deuterium, also known as heavy hydrogen), and tritium (T, also known as superheavy hydrogen); the isotopes of oxygen comprise ¹⁶O, ¹⁷O and ¹⁸O; the isotopes of sulfur comprise ³²S, ³³S, ³⁴S and ³⁶S; the isotopes of nitrogen comprise ¹⁴N and ¹⁵N; the isotope of fluorine comprises ¹⁹F; the isotopes of chlorine comprise ³⁵Cl and ³⁷Cl; and the isotopes of bromine comprise ⁷⁹Br and ⁸¹Br.

The term “halogen” herein refers to F, Cl, Br, I, or isotopes thereof.

The term “halo” or “substituted with halogen” refers to being substituted with one or more groups selected from F, Cl, Br, I, or isotopes thereof, wherein the upper limit of the number of halogen substituents is equal to the sum of the number of hydrogens that can be substituted in the group to be substituted. Without particular limitation, the number of halogen substituents is any integer between 1 and the upper limit, and when the number of halogen substituents is greater than 1, the group to be substituted can be substituted with the same or different halogen. Generally, the circumstances of being substituted with 1-5 halogen, 1-3 halogen, 1-2 halogen, and 1 halogen are included.

The term “deuterium” refers to the isotope deuterium of hydrogen (H).

The term “deuterated” or “deuterated material” refers to the case where a hydrogen atom on a group, such as alkyl, cycloalkyl, alkylene, aryl, heteroaryl, mercapto, heterocycloalkyl, alkenyl and alkynyl is substituted with at least one deuterium atom, wherein the upper limit of the number of deuterium substituents is equal to the sum of the number of hydrogens that can be substituted in the group to be substituted. Without particular limitation, the number of deuterium substituents is any integer between 1 and the upper limit, for example, 1-20 deuterium atoms, 1-10 deuterium atoms, 1-6 deuterium atoms, 1-3 deuterium atoms, 1-2 deuterium atoms or 1 deuterium atom.

Group “C_{x-y}” refers to a group comprising x to y carbon atoms, for example, “C₁₋₆ alkyl” refers to alkyl comprising 1-6 carbon atoms.

The term “alkyl” refers to a monovalent straight or branched saturated aliphatic hydrocarbon group, usually an alkyl group with 1 to 20 carbon atoms, or an alkyl group with 1 to 8 carbon atoms, or an alkyl group with 1 to 6 carbon atoms, or an alkyl group with 1 to 4 carbon atoms, such as “C₁₋₆ alkyl”, “C₁₋₅ alkyl”, “C₁₋₄ alkyl”, “C₁₋₃ alkyl”, “C₁₋₂ alkyl”, “C₂₋₆ alkyl”, “C₂₋₅ alkyl”, “C₂₋₄ alkyl”, “C₂₋₃ alkyl”, “C₃₋₆ alkyl”, “C₃₋₅ alkyl”, “C₃₋₄ alkyl”, “C₄₋₆ alkyl”, “C₄₋₅ alkyl”, and “C₅₋₆ alkyl”. Non-limiting examples of alkyl include methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc. The alkyl can be further substituted with any substituent.

The term “alkylene” refers to a bivalent straight or branched saturated alkyl. Examples of alkylene include, but are not limited to, methylene, ethylidene, etc. The alkylene can be optionally further substituted with a substituent.

The term “haloalkyl” refers to an alkyl group in which one or more hydrogens are substituted with one or more halogen atoms (e.g., fluorine, chlorine, bromine, iodine, or isotopes thereof), wherein the upper limit of the number of halogen substituents is equal to the sum of the number of hydrogens that can be substituted in the alkyl group. Without particular limitation, the number of halogen substituents is any integer between 1 and the upper limit. Generally, the alkyl group is substituted with 1-5 halogen, 1-3 halogen, 1-2 halogen or 1 halogen; and when the number of halogen substituents is greater than 1, the group to be substituted can be substituted with the same or different halogen. Specific examples include, but are not limited to, —CF₃, —CH₂Cl, —CH₂CF₃, —CCl₂, CF₃, etc.

The term “alkoxy” or “alkyloxy” refers to —O-alkyl, such as —O—C₁₋₈ alkyl, —O—C₁₋₆ alkyl, —O—C₁₋₄ alkyl or —O—C₁₋₂ alkyl. Non-limiting and specific examples of alkoxy or alkyloxy include methoxy, ethoxy, n-propoxy, isopropoxy, n-butoxy, sec-butoxy, tert-butoxy, n-pentoxy, n-hexyloxy, cyclopropoxy, cyclobutoxy, etc. The alkoxy may be optionally substituted with a substituent.

The term “haloalkoxy” refers to —O-haloalkyl, such as —O-halo C₁₋₈ alkyl, —O-halo C₁₋₆ alkyl, —O-halo C₁₋₄ alkyl or —O-halo C₁₋₂ alkyl; the upper limit of the number of halogen substituents is equal to the sum of the number of hydrogens that can be substituted in the group to be substituted. Without particular limitation, the number of halogen substituents is any integer between 1 and the upper limit, preferably 1-5 halogen, 1-3 halogen, 1-2 halogen, and 1 halogen; and when the number of halogen substituents is greater than 1, the group to be substituted can be substituted with the same or different halogen. Non-limiting examples of haloalkoxy include monofluoromethoxy, difluoromethoxy, trifluoromethoxy, difluoroethyloxy, etc.

The term “alkenyl” refers to a straight or branched hydrocarbon group comprising at least one carbon-carbon double bond (C═C) and generally comprises 2 to 18 carbon atoms, such as 2 to 8 carbon atoms, further such as 2 to 6 carbon atoms, and more further such as 2 to 4 carbon atoms. Examples of alkenyl include, but are not limited to vinyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc.; and the alkenyl may further be optionally substituted with a substituent.

The term “alkenylene” refers to a straight or branched divalent unsaturated hydrocarbon group containing at least one carbon-carbon double bond (C═C). Unless otherwise specified, the alkynylene contains 2-6 carbon atoms, preferably 2-4 carbon atoms. Non-limiting examples of alkynylene include ethynylene, and the alkenylene may be optionally substituted with a substituent.

The term “alkynyl” refers to a straight or branched hydrocarbon group containing at least one carbon-carbon triple bond (C═C) and generally comprises 2 to 18 carbon atoms; further, alkynyl comprises 2 to 8 carbon atoms; further, alkynyl comprises 2 to 6 carbon atoms, and more further, alkynyl comprises 2 to 4 carbon atoms. Examples of alkynyl include, but are not limited to ethynyl, 1-propynyl, 2-propynyl, butynyl, 2-butynyl, 3-butynyl, 1-methyl-2-propynyl, 4-pentynyl, 3-pentynyl, 1-methyl-2-butynyl, 2-hexynyl, 3-hexynyl, 2-heptynyl, 3-heptynyl, 4-heptynyl, 3-octynyl, 3-nonynyl, 4-decynyl, etc.; and the alkynyl may be optionally substituted with a substituent.

The term “alkynylene” refers to a straight or branched, divalent unsaturated hydrocarbon group containing a carbon-carbon triple bond (C═C) and generally comprises 2-6 carbon atoms, and further comprises 2-4 carbon atoms. Non-limiting examples of alkynylene include ethynylene, propynylene, and butynylene, and the alkynylene may be optionally substituted with a substituent.

The term “cycloalkyl” refers to a saturated or partially unsaturated, non-aromatic carbocyclic hydrocarbon group containing no ring heteroatoms. The cycloalkyl may be monocyclic, bicyclic or polycyclic; the bicyclic or polycyclic cycloalkyl may be in the form of a fused ring, a spiro ring, a bridged ring or a combination thereof, and may comprise one or more aromatic rings, but the ring system is non-aromatic as a whole; and the connection site is on a non-aromatic ring. Generally, the cycloalkyl contains 3 to 20 carbon atoms; further, the cycloalkyl contains 3-8 carbon atoms; and still further, the cycloalkyl contains 3-6 carbon atoms; when the cycloalkyl is monocyclic cycloalkyl, the cycloalkyl contains 3-15 carbon atoms, or 3-10 carbon atoms, or 3-8 carbon atoms, or 3-6 carbon atoms; when the cycloalkyl is bicyclic or polycyclic cycloalkyl, the cycloalkyl contains 5-12 carbon atoms, or 5-11 carbon atoms, or 6-10 carbon atoms. Non-limiting examples of cycloalkyl include cyclopropyl, cyclobutyl, cyclopentyl, cyclohexyl, cycloheptyl, butenyl, cyclopentenyl, cyclohexenyl,

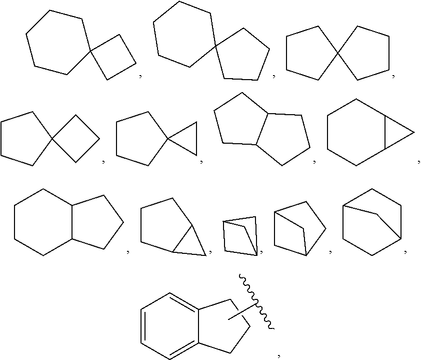

etc., and the cycloalkyl may be optionally substituted with a substituent.

The term “cycloalkylene” refers to a divalent group of cycloalkyl.

The term “aryl” refers to a substituted or unsubstituted aromatic 5- to 15-membered carbocycle, and includes monocyclic aryl and fused aryl. Aryl is preferably a 5- to 10-membered aromatic ring, further preferably a 5- to 9-membered aromatic ring, and further preferably a 5- to 8-membered aromatic ring. Aryl ring can be fused to an aryl ring and a non-aryl ring (such as a heteroaryl, heterocycloalkyl, or cycloalkyl ring), wherein the aryl ring is the connection site. Non-limiting examples of aryl include phenyl, naphthyl, anthryl, phenanthryl and

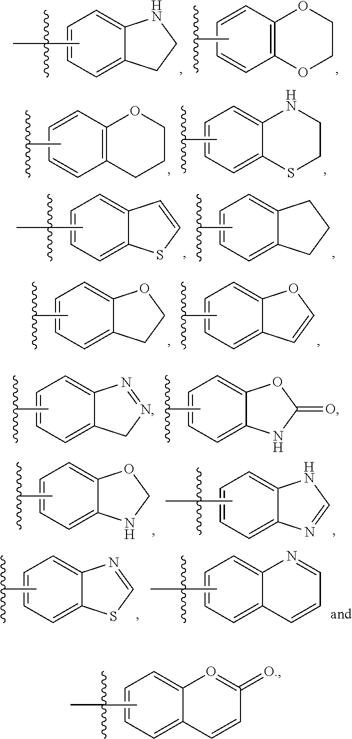

and the aryl may be optionally further substituted with any substituent.

The term “heterocycloalkyl” refers to a saturated or partially unsaturated, non-aromatic carbocycle comprising 1, 2, 3, 4 or 5 heteroatoms selected from N, S, O, P or Si. The heterocycloalkyl may be monocyclic, bicyclic or polycyclic; the bicyclic or polycyclic heterocycloalkyl may be in the form of a bridged ring, a fused ring, a spiro ring or a combination thereof, and may comprise one or more aromatic rings or heteroaromatic rings, but the ring system is non-aromatic as a whole; and the connecting site is on a non-aromatic ring. Usually, the heterocycloalkyl is a 3-20-membered ring. When the heterocycloalkyl is monocyclic heterocycloalkyl, the heterocycloalkyl is usually a 3- to 15-membered ring, or a 3- to 10-membered ring, or a 3-8-membered ring, or a 3-6-membered ring; when the heterocycloalkyl is bicyclic or polycyclic heterocycloalkyl, the heterocycloalkyl is usually a 5- to 12-membered ring, or a 5- to 11-membered ring, or a 6- to 9-membered ring. The heteroatoms N, S and P include their oxidation states C═O, N—O, S═O, S(═O)₂, P═O, and P(═O)₂. When heterocycloalkyl is a bicyclic or polycyclic ring, at least one ring contains at least one heteroatom, and the heterocycloalkyl can be a bicyclic or polycyclic ring formed by a ring containing the heteroatom(s) and a ring containing no heteroatom, or a bicyclic or polycyclic ring formed by a ring containing the heteroatom(s) and a ring containing the heteroatom(s). When heterocycloalkyl is connected to other groups, the connection site can be at a heteroatom or a carbon atom. Non-limiting examples of heterocycloalkyl include azetidinyl, morpholinyl, piperazinyl, piperidyl, tetrahydropyranyl, oxetanyl, pyranyl, azacyclopentenyl, azacyclohexenyl, oxacyclopentenyl, oxacyclohexenyl, etc., and the heterocycloalkyl may be optionally substituted with a substituent.

The term “heteroaromatic ring” or “heteroaryl”, unless otherwise specified, refers to an aromatic ring containing 1 to 5 heteroatoms selected from N, O, S, P, Si and their oxidation states, which can be monocyclic, bicyclic, or polycyclic, wherein the bicyclic or polycyclic heteroaromatic ring or heteroaryl can be bridged rings, fused rings, spiro rings and combinations thereof. Bicyclic or polycyclic heteroaromatic ring or heteroaryl can be formed by fusion of heteroaryl to aryl, of heteroaryl to heteroaryl, or of heteroaryl to cycloalkyl or heterocycloalkyl, wherein the heteroaryl is the connection site. Non-limiting examples of heteroaromatic ring or heteroaryl include furyl, thienyl, pyrrolyl, oxazolyl, thiazolyl, imidazolyl, pyrazolyl, pyridyl, pyrimidinyl, pyridazinyl, pyrazinyl, indolyl, purinyl,

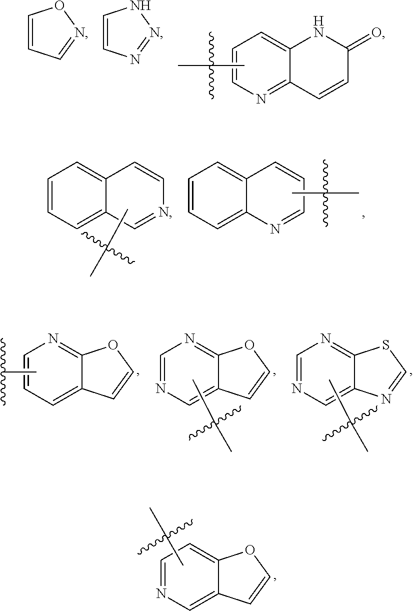

etc. The heteroaryl may be optionally substituted with a substituent.

The term “aromatic ring” refers to an aromatic ring system containing or containing no N, S, O, P, Si and other heteroatoms, and its definition includes aryl and heteroaryl. The aromatic ring may be optionally substituted with a substituent.

The term “heterocycle” or “heterocyclyl” refers to a saturated or unsaturated, aromatic or non-aromatic ring containing 1 to 5 heteroatoms selected from N, O, S, P, Si and their oxidation states, and its meaning includes heteroaryl and heterocycloalkyl. Heterocycles include monocyclic heterocycles, bicyclic bridged heterocycles, bicyclic fused heterocycles and bicyclic spiro heterocycles or combinations thereof. The heterocycle is usually a 3- to 12-membered heterocycle, or a 5- to 12-membered heterocycle, or a 5- to 7-membered heterocycle. Heterocyclyl can be connected to a heteroatom or a carbon atom. Non-limiting examples of heterocyclyl include oxiranyl, azacyclopropyl, oxetanyl, azetidinyl, 1,3-dioxolanyl, 1,4-dioxolanyl, 1,3-dioxanyl, piperazinyl, azacycloheptyl, pyridyl, furyl, thienyl, pyranyl, N-alkylpyrrolyl, pyrimidyl, pyrazinyl, pyrazolyl, pyridazinyl, imidazolyl, piperidyl, piperadinyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, oxazolyl, dihydrooxazolyl, tetrahydrooxazolyl, tetrahydrothiazolyl, tetrahydropyranyl, benzoimidazolyl, benzopyridyl, pyrrolopyridyl, benzodihydrofuryl, azabicyclo[3.2.1]octanyl, azabicyclo[5.2.0]nonanyl, oxatricyclo[5.3.1.1]dodecyl, azaadamantyl and oxaspiro[3.3]heptanyl,

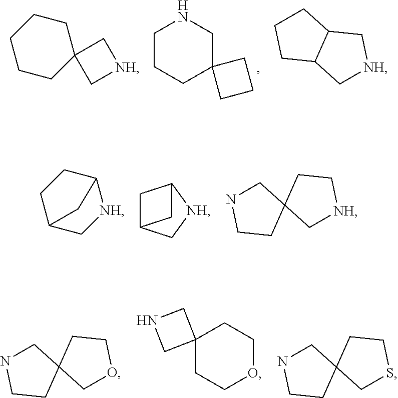

etc., and the heterocycle may be optionally substituted with a substituent.

The term “spiro ring” refers to a polycyclic group sharing one carbon atom (referred to as a spiro atom) between rings, which may contain 0 or at least 1 double or triple bond, and may contain 0 to 5 heteroatoms selected from N, O, S, P, Si and their oxidation states. Generally, a spiro ring is a 5- to 14-membered ring, or a 5- to 12-membered ring, or a 5- to 10-membered ring. Generally, a spiro ring is a spiro ring formed by a three-membered ring and a three-membered ring, a three-membered ring and a four-membered ring, a three-membered ring and a five-membered ring, a three-membered ring and a six-membered ring, a four-membered ring and a four-membered ring, a four-membered ring and a five-membered ring, a four-membered ring and a six-membered ring, a five-membered ring and a five-membered ring or a five-membered ring and a six-membered ring. The spiro ring may be spirocyclic, non-limiting examples of which include

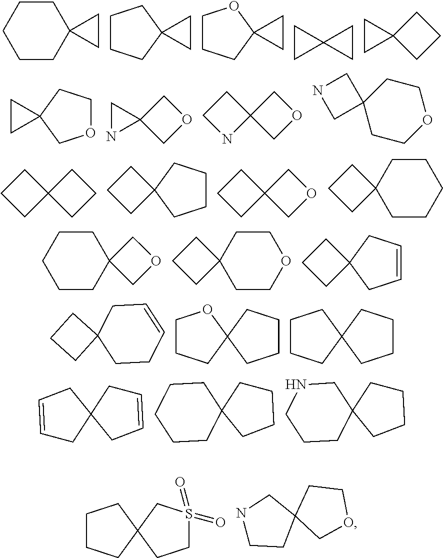

and the spiro ring may be optionally substituted with a substituent.

The term “bicyclic spiro cycloalkyl” means that both rings forming the spiro ring are cycloalkyl.

The term “bicyclic spiro heterocycloalkyl” means that at least one of the two rings forming the spiro ring is heterocycloalkyl.

The term “bicyclic heteroaromatic ring” refers to an aromatic bicyclic system as a whole, and at least one of the rings in the bicyclic system is a ring containing a heteroatom.

The term “fused ring” refers to a polycyclic group in which the rings share two adjacent ring atoms and one chemical bond, which may contain one or more double or triple bonds, and may contain 0 to 5 heteroatoms selected from N, S, O, P, Si and their oxidation states. Generally, a fused ring is a 5- to 20-membered ring, or a 5- to 14-membered ring, or a 5- to 12-membered ring or a 5- to 10-membered ring. Generally, a fused ring is in the form of a three-membered ring fused a four-membered ring (indicating a fused ring formed by a three-membered ring and a four-membered ring, and either the three-membered ring or the four-membered ring may be possibly used as the basic ring according to the IUPC nomenclature; similarly hereinafter), a three-membered ring fused a five-membered ring, a three-membered ring fused a six-membered ring, a four-membered ring fused a four-membered ring, a four-membered ring fused a five-membered ring, a four-membered ring fused a six-membered ring, a five-membered ring fused a five-membered ring, a five-membered ring fused a six-membered ring, and a six-membered ring fused a six-membered ring. Non-limiting examples of fused ring include purine, quinoline, isoquinoline, benzopyran, benzofuran, benzothiophene, and

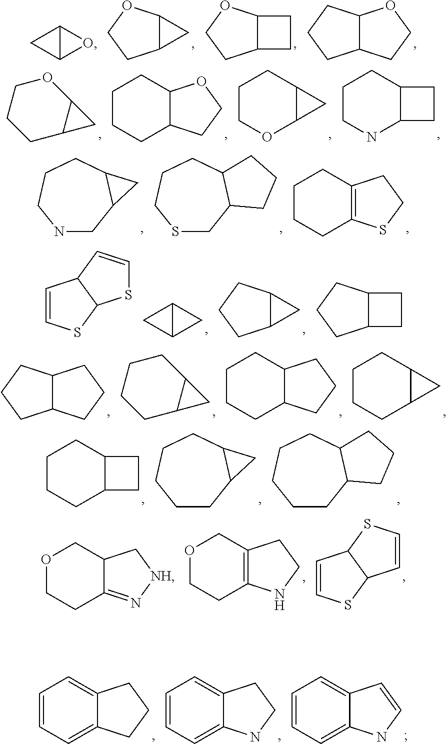

and the fused ring may be optionally substituted with a substituent.

The term “bridged ring” refers to a ring system in which two non-adjacent ring atoms are shared between two rings and a bridged ring may contain 1 or more double or triple bonds. The bridged ring may contain 0 to 5 heteroatoms selected from N, S, O, P, Si and their oxidation states. Generally, the bridged ring has 5 to 20, or 5 to 14, or 5 to 12, or 5 to 10 ring atoms. Non-limiting examples of bridged ring include adamantane,

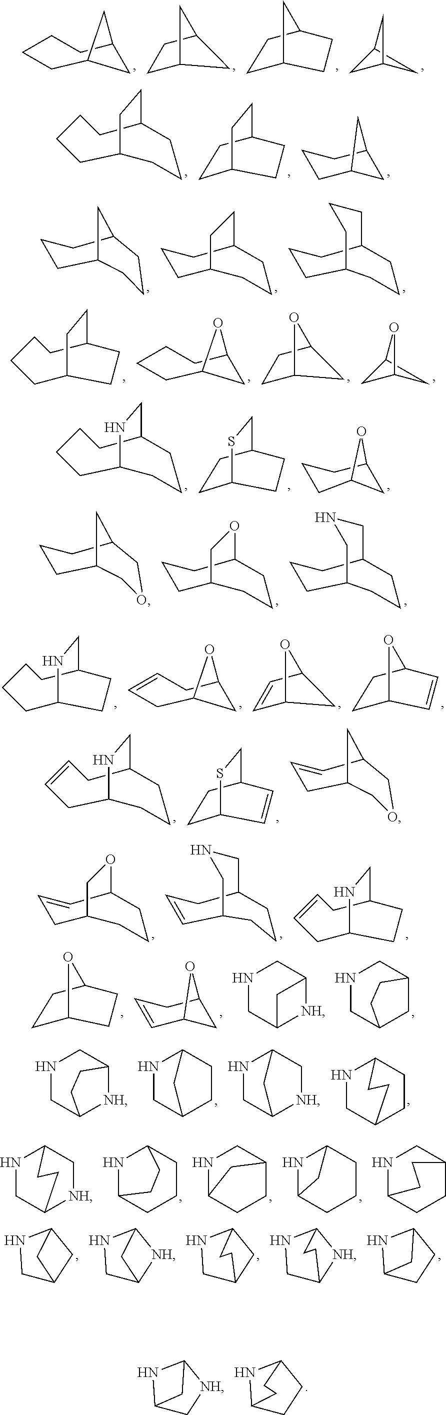

Unless otherwise specified, the term “substitute” or “substituent” refers to any substitution at a position allowed by chemical theory, and the number of substituents conforms to the rules of chemical bonding. Exemplary substituents include, but are not limited to: C₁₋₆ alkyl, C₂₋₆ alkenyl, C₂₋₆ alkynyl, C₃₋₈ heteroalkyl, C₅₋₁₂ aryl, 5- to 12-membered heteroaryl, hydroxyl, C₁₋₆ alkoxy, C₅₋₁₂ aryloxy, thiol, C₁₋₆ alkylthio, cyano, halogen, C₁₋₆ alkylthiocarbonyl, C₁₋₆ alkylcarbamoyl, N-carbamoyl, nitro, silyl, sulfinyl, sulfonyl, sulfoxide, halo C₁₋₆ alkyl, halo C₁₋₆ alkoxy, amino, phosphonic acid, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, etc.

The term “optional” or “optionally” refers to that the events or circumstances subsequently described may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

The term “pharmaceutically acceptable salt” refers to a salt of the compound of the present invention, which salt maintains the biological effectiveness and characteristics of a free acid or a free base and is obtained by reacting the free acid with a non-toxic inorganic base or organic base, or reacting the free base with a non-toxic inorganic acid or organic acid.

The term “pharmaceutical composition” represents a mixture of one or more compounds described herein or the stereoisomers, solvates, or pharmaceutically acceptable salts thereof and other components comprising physiologically/pharmaceutically acceptable carriers and/or excipients.

The term “carrier” refers to: a system that does not cause significant irritation to the organism and does not eliminate the biological activity and characteristics of the administered compound, and can change the way the drug enters the human body and the distribution of the drug in the body, control the release rate of the drug and delivery the drug to targeted organs. Non-limiting examples of the carrier include microcapsule, microsphere, nanoparticle, liposome, etc.

The term “excipient” refers to: a substance that is not a therapeutic agent per se, but used as a diluent, adjuvant, adhesive and/or vehicle for addition to a pharmaceutical composition, thereby improving the disposal or storage properties thereof, or allowing to or promoting the formation of a compound or a pharmaceutical composition into a unit dosage form for administration. As is known to those skilled in the art, a pharmaceutically acceptable excipient can provide various functions and can be described as a wetting agent, a buffer, a suspending agent, a lubricant, an emulsifier, a disintegrating agent, an absorbent, a preservative, a surfactant, a colorant, a flavoring agent and a sweetening agent. Examples of pharmaceutically acceptable excipients include, but are not limited to: (1) sugars, such as lactose, glucose and sucrose; (2) starch, such as corn starch and potato starch; (3) cellulose and its derivatives, such as sodium carboxymethyl cellulose, ethyl cellulose, cellulose acetate, hydroxypropyl methylcellulose, hydroxypropyl cellulose, microcrystalline cellulose and croscarmellose (such as croscarmellose sodium); (4) tragacanth powder; (5) malt; (6) gelatin; (7) talc; (8) excipients, such as cocoa butter or suppository wax; (9) oils, such as peanut oil, cottonseed oil, safflower oil, sesame oil, olive oil, corn oil and soybean oil; (10) diols, such as propylene glycol; (11) polyols, such as glycerin, sorbitol, mannitol and polyethylene glycol; (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) pH buffered solution; (21) polyester, polycarbonate and/or polyanhydride; and (22) other non-toxic compatible substances used in a pharmaceutical preparation.

The term “stereoisomer” refers to an isomer produced as a result of different spatial arrangement of atoms in molecules, including cis-trans isomers, enantiomers and conformational isomers.

The compounds of the present invention also include tautomers thereof, for example, when the present invention describes the left side compound in which the pyrimidine ring is substituted with OH, the right side tautomer compound is also included.

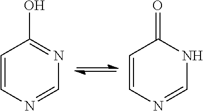 [SMILES: Oc1ccncn1 ; O=c1ccnc[nH]1]

The term “solvate” refers to a substance formed by the compound of the present invention or the salt thereof and a stoichiometric or non-stoichiometric solvent bound by intermolecular non-covalent forces. When the solvent is water, the solvate is a hydrate.

The term “cocrystal” refers to a crystal formed by the combination of active pharmaceutical ingredient (API) and co-crystal former (CCF) under the action of hydrogen bonds or other non-covalent bonds. The pure state of API and CCF are both solid at room temperature, and there is a fixed stoichiometric ratio among various components. The cocrystal is a multi-component crystal, which includes both a binary cocrystal formed between two neutral solids and a multi-element cocrystal formed between a neutral solid and a salt or solvate.

### DETAILED DESCRIPTION OF EMBODIMENTS

The content of the present invention is described in detail with the following examples. If a specific condition is not indicated in the examples, a conventional condition is used in an experimental method. The listed examples are intended to better illustrate the content of the present invention, but should not be construed as limiting the content of the present invention. According to the above-mentioned content of the invention, those skilled in the art can make unsubstantial modifications and adjustments to the embodiments, which still fall within the protection scope of the present invention.

### Detection Method

The structure of the compound is determined by nuclear magnetic resonance (NMR) or (and) mass spectrometry (MS). The NMR shift (δ) is given in the unit of 10-6 (ppm). NMR is determined with Bruker Avance III 400 and Bruker Avance 300; the solvents for determination are deuterated dimethyl sulfoxide (DMSO-d6), deuterated chloroform (CDCl3) and deuterated methanol (CD3OD); and the internal standard is tetramethylsilane (TMS);
MS is determined with Agilent 6120B (ESI) and Agilent 6120B (APCI);
HPLC is determined with Agilent 1260DAD high pressure liquid chromatograph (Zorbax SB-C18 100×4.6 mm, 3.5 μM);
Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
and for the column chromatography, Yantai Huanghai silica gel of 200-300 mesh silica gel is generally used as a carrier.

### Example 1

### N-methyl-5-(4-((6-oxo-7-vinyl-5,6-dihydro-1,5-naphthyridin-3-yl)methyl)piperazin-1-yl)picolinamide (Compound 1)

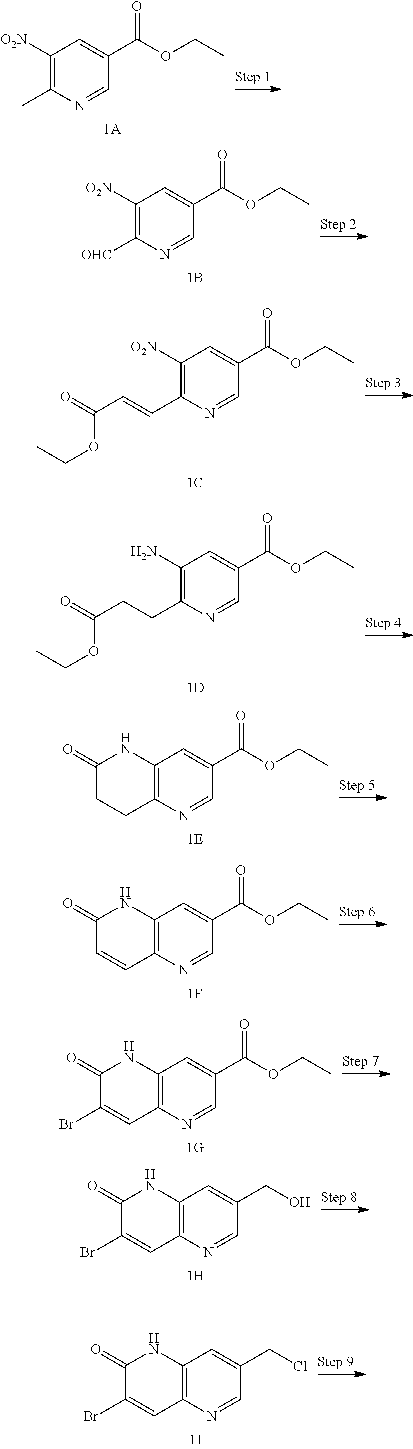

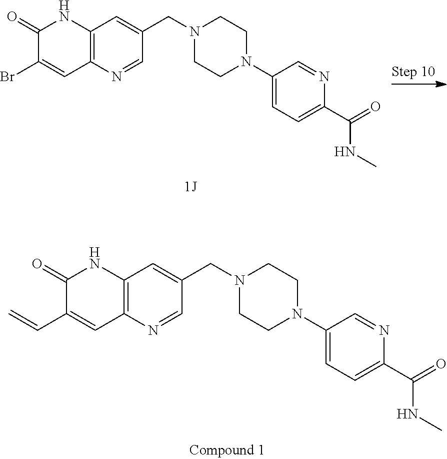

### Step 1:

Compound 1A (20 g, 95.21 mmol) and selenium dioxide (21.13 g, 190.42 mmol) were added to 1,4-dioxane solvent (200 mL), and the mixture was heated to reflux, stirred for 3 hours, cooled, filtered, and directly purified and separated by silica gel column chromatography (petroleum ether:ethyl acetate (v/v)=1:0-5:1), to afford the title compound (20 g, yield: 94%).

LCMS m/z=225.1 [M+1]⁺

¹H NMR (400 MHz, CDCl₃) δ 10.31 (s, 1H), 9.50 (d, 1H), 8.80 (d, 1H), 4.52 (q, 2H), 1.47 (t, 3H).

### Step 2:

Under nitrogen atmosphere, sodium hydride (8.9 g, 223.23 mmol) was slowly added to anhydrous tetrahydrofuran solvent, and the mixture was cooled to 0° C. in an ice bath. Ethyl 2-(diethoxyphosphoryl)acetate (50 g, 223.23 mmol) was then slowly added dropwise to the above-mentioned solution, and the mixture was reacted for half an hour. Subsequently, the solution was cooled to −78° C., reagent 1B (20 g, 89.29 mmol) dissolved in anhydrous tetrahydrofuran was slowly added dropwise to the above-mentioned reaction liquid, and the mixture was reacted for 1 hour. After the reaction was completed, the reaction liquid was quenched with saturated ammonium chloride solution, extracted with ethyl acetate, dried over anhydrous sodium sulfate and filtered, and the filtrate was concentrated under reduced pressure and separated by silica gel column chromatography (petroleum ether:ethyl acetate (v/v)=1:0-3:1), to afford the title compound (15 g, yield: 57%).

LCMS m/z=295.1 [M+1]⁺

¹H NMR (400 MHz, DMSO-d6) δ 9.32 (d, 1H), 8.79 (d, 1H), 8.07 (d, 1H), 7.08 (d, 1H), 4.42 (q, 2H), 4.25 (q, 2H), 1.37 (t, 3H), 1.28 (t, 3H).

### Step 3:

Reagent 1C (15 g, 51.02 mmol) was added to ethanol (200 mL), palladium on carbon was added, and the mixture was subjected to hydrogen replacement and reacted at room temperature under hydrogen atmosphere for 16 hours. After the reaction was completed, the reaction liquid was filtered with diatomaceous earth, to afford the title compound (12 g, yield: 92%).

LCMS m/z=267.1 [M+1]⁺

¹H NMR (400 MHz, DMSO-d6) δ 8.21 (d, 1H), 7.45 (d, 1H), 5.44 (s, 2H), 4.29 (d, 2H), 4.05 (d, 2H), 2.86 (d, 2H), 2.75 (d, 2H), 1.30 (t, 3H), 1.16 (t, 3H).

### Step 4:

Reagent 1D (12 g, 45.11 mmol) was added to hydrogen chloride/1,4-dioxane solution (200 mL), and the mixture was reacted at room temperature for 1 hour. After the reaction was completed, the reaction liquid was directly concentrated, to afford the title compound (9 g, yield: 91%).

LCMS m/z=221.1 [M+1]⁺

### Step 5:

Reagent 1E (9 g, 40.91 mmol) and dichlorodicyanobenzoquinone (11.2 g, 49.09 mmol) were added to 1,4-dioxane (200 mL), and the mixture was reacted at reflux for 2 hours. After the reaction was completed, saturated aqueous sodium bicarbonate solution was added, and the mixture was stirred for half an hour, filtered, and washed with saturated aqueous sodium bicarbonate solution, water and ether to afford a solid, which was spun to dryness to afford the title compound (8 g, yield: 90%).

LCMS m/z=219.1 [M+1]⁺

¹H NMR (400 MHz, DMSO-d6) δ 12.03 (s, 1H), 8.90 (d, 1H), 8.18 (d, 1H), 7.99 (d, 1H), 6.88 (d, 1H), 4.39 (q, 2H), 1.36 (t, 3H).

### Step 6:

Reagent 1F (4 g, 18.18 mmol) and sodium bromate (5.49 g, 36.36 mmol) were added to a solution of hydrogen bromide in acetic acid (50 mL), and the mixture was reacted at 100° C. for 16 hours. After the reaction was completed, the reaction liquid was concentrated, washed with saturated sodium bicarbonate, extracted and concentrated, to afford a crude product (7 g).

LCMS m/z=297.0 [M+1]⁺

¹H NMR (400 MHz, DMSO-d6) δ 11.04 (s, 1H), 8.94 (d, 1H), 8.58 (s, 1H), 8.21 (d, 1H), 4.39 (q, 2H), 1.36 (t, 3H).

### Step 7:

Reagent 1G (2 g, crude) was added to tetrahydrofuran solvent (70 mL), diisobutylaluminum hydride (32 ml, 1.5 mol/L in toluene) was slowly added in an ice bath, and the mixture was reacted at this temperature for 2 hours. After the reaction was completed, the reaction liquid was quenched with water and concentrated. The crude product was eluted with dichloromethane/methanol=10:1, concentrated, and purified by reverse-phase HPLC (H₂O:CH₃CN=1:0-10:1), to afford the title compound (400 mg, 33%).

LCMS m/z=255.0 [M+1]⁺

### Step 8:

Reagent 1H (400 mg) was added to dry dichloromethane solvent (70 mL), thionyl chloride (32 ml, 1.5 mol/L in toluene) was slowly added in an ice bath, 2 drops of DMF was added dropwise, and the mixture was stirred overnight at room temperature. After the reaction was completed, the reaction liquid was concentrated to dryness, to afford the title compound (420 mg, 100%).

LCMS m/z=273.0 [M+1]⁺

### Step 9:

Raw material 1| (150 mg, 0.58 mmol), N-methyl-5-(piperazin-1-yl)picolinamide (158 mg, 0.58 mmol), DIPEA (0.48 mL, 2.9 mmol) and potassium iodide (190 mg, 1.16 mmol) were added to DMF (6 mL) solvent, and then the mixture was heated to 70° C. and reacted for 6 hours. The reaction liquid was filtered, and the filtrate was concentrated under reduced pressure to dryness and purified by preparative TLC (DCM:MeOH=10:1), to afford a target compound (100 mg, yield: 38%).

LCMS m/z=457.1 [M+1]⁺

### Step 10:

Reagent 1J (50 mg, 0.11 mmol), tributylvinyltin (67 mg, 0.22 mmol), potassium carbonate (15 mg, 0.11 mmol), tetraethylammonium chloride (24 mg, 0.22 mmol), and bis(triphenylphosphine)palladium (II) dichloride (77 mg, 0.11 mmol) were added to dry DMF solvent (10 mL), and under nitrogen protection, the mixture was heated to 100° C., stirred for 5 hours, cooled and filtered. The filtrate was concentrated under reduced pressure and purified by preparative TLC (DCM:MeOH=10:1), to afford the title compound (5 mg, 11%).

LCMS m/z=405.2 [M+1]⁺

¹H NMR (400 MHz, CD₃OD) δ8.48 (d, 1H), 8.18 (d, 1H), 7.80 (d, 1H), 7.67 (s, 1H), 7.47-7.54 (m, 1H), 7.25-7.28 (m, 1H), 6.87 (t, 1H), 6.10 (d, 1H), 5.60 (d, 1H), 3.66 (s, 2H), 3.30-3.32 (m, 4H), 2.83 (s, 3H),2.58-2.60 (m, 4H).

### Example 2

### 5-(4-((7-cyclopropyl-6-oxo-5,6-dihydro-1,5-naphthyridin-3-yl)methyl)piperazin-1-yl)-N-methylpicolinamide (Compound 2)

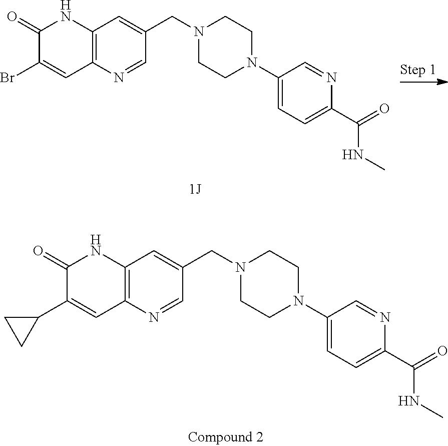 [SMILES: CNC(=O)c1ccc(N2CCN(Cc3cnc4cc(Br)c(=O)[nH]c4c3)CC2)cn1 ; CNC(=O)c1ccc(N2CCN(Cc3cnc4cc(C5CC5)c(=O)[nH]c4c3)CC2)cn1]

To a reaction flask were added 1J (300 mg, 0.66 mmol), cyclopropylboronic acid (70 mg, 0.81 mmol), cesium carbonate (430 mg, 1.32 mmol) and dioxane (6 mL), the reaction mixture was subjected to nitrogen replacement for protection, and then Pd(dppf)Cl₂ (90 mg, 0.12 mmol) was added. After the addition was completed, the mixture was heated to 100° C. and reacted for 4 h. After the reaction was completed, to the reaction liquid was added water (20 mL), and then the mixture was extracted with ethyl acetate (50 mL). The organic phase was separated, and the aqueous phase was extracted with ethyl acetate (50 mL×2). The organic phases were combined, washed with saturated brine (50 mL×2), dried over anhydrous sodium sulfate, filtered and concentrated, to afford a crude, which was purified by column chromatography (eluent: dichloromethane:methanol=10:1), to afford a product (60 mg). The product was further purified by preparative HPLC (Preparation method: instrument: waters 2767 (preparative liquid phase chromatographic instrument); chromatographic column: SunFire@ Prep C18 (19 mm×250 mm); the sample was dissolved in DMF and filtered with a 0.45 um filter to prepare a sample liquid; Preparative chromatography conditions: a. composition of mobile phases A and B: mobile phase A: acetonitrile, and mobile phase B: water (containing 5 mM ammonia water); b. gradient elution, mobile phase A: 25%- 85%; c. flow rate: 15 mL/min; d. elution time: 15 min; retention time: 6.0 min), to afford compound 2 (18 mg).

LCMS m/z=419.3 [M+1]⁺

¹H NMR (400 MHz, CD₃OD) δ 8.47 (d, 1H), 8.29 (d, 1H), 7.91 (d, 1H), 7.77 (s, 1H), 7.52 (s, 1H), 7.40-7.36 (m, 1H), 3.75 (s, 2H), 3.43-3.41 (m, 4H), 2.95 (s, 3H), 2.71-2.68 (m, 4H), 2.23-2.19 (m, 1H), 1.11-1.07 (m, 2H), 0.86-0.84 (m, 2H).

### Example 3

### N-cyclopropyl-1′-((7-cyclopropyl-6-oxo-5,6-dihydro-1,5-naphthyridin-3-yl)methyl)-1′,2′,3′,6′-tetrahydro-[3,4′-bipyridine]-6-carboxamide (Compound 3)

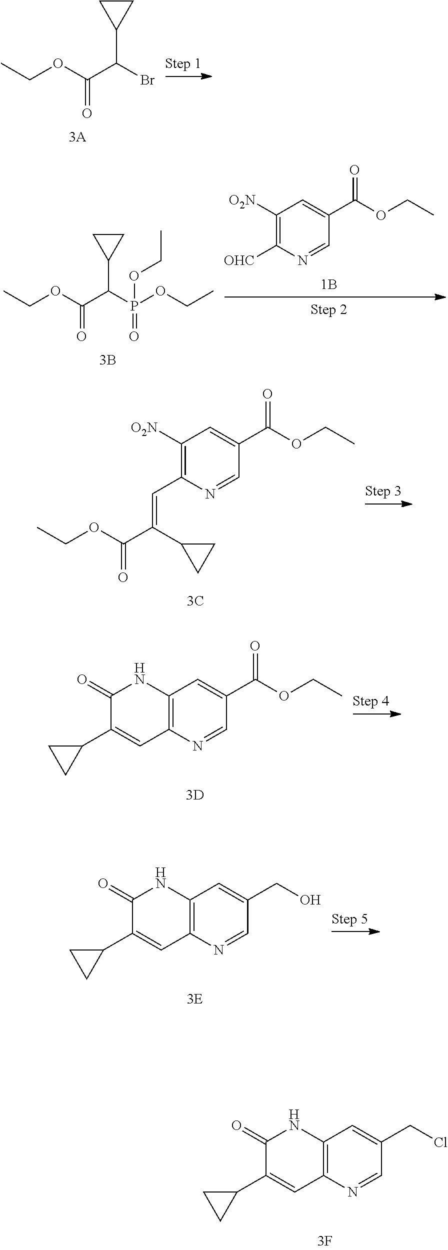

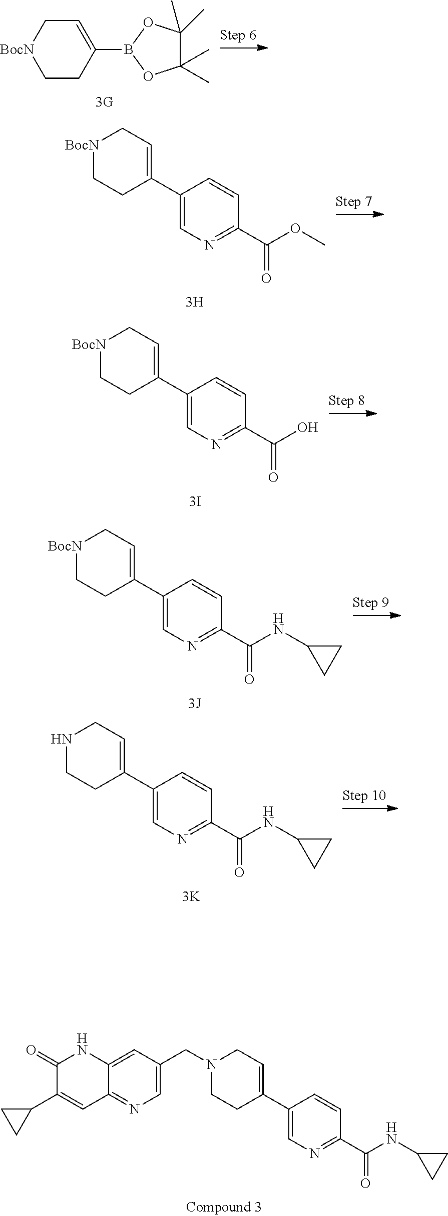 [SMILES: CC(C)(C)OC(=O)N1CC=C(B2OC(C)(C)C(C)(C)O2)CC1 ; COC(=O)c1ccc(C2=CCN(C(=O)OC(C)(C)C)CC2)cn1 ; CC(C)(C)OC(=O)N1CC=C(c2ccc(C(=O)O)nc2)CC1 ; CC(C)(C)OC(=O)N1CC=C(c2ccc(C(=O)NC3CC3)nc2)CC1 ; O=C(NC1CC1)c1ccc(C2=CCNCC2)cn1 ; O=C(NC1CC1)c1ccc(C2=CCN(Cc3cnc4cc(C5CC5)c(=O)[nH]c4c3)CC2)cn1]

### Step 1:

Ethyl 2-bromo-2-cyclopropylacetate 3A (30.0 g, 144.89 g) was weighed and dissolved in triethyl phosphite (150 mL), and then the mixture was heated to 130° C. in a sealed tube and reacted for 20 h. After the reaction was completed, the reaction liquid was cooled to room temperature and subjected to rotary evaporation to remove excess triethyl phosphite, to afford crude target compound 3B (38.29 g, yield: 100.00%), which was directly used in the subsequent reaction.

LC-MS (ESI): m/z=265.2 [M+H]⁺.

### Step 2:

NaH (9.28 g, 231.96 mmol, 60%) was weighed and dissolved in ultra-dry tetrahydrofuran (300 mL), and then the mixture was cooled to about −10° C. 3B (38.29 g, 144.97 mmol) was weighed and dissolved in tetrahydrofuran (50 mL), and then the solution of 3B in THF was slowly added to the reaction liquid. After the dropwise addition was completed, the mixture was slowly naturally warmed to room temperature and stirred for 30 min. After the reaction was completed, the solution was cooled to −70° C., 1B (13.0 g, 57.99 mmol) was then dissolved in ultra-dry tetrahydrofuran (50 mL), and the solution was slowly added dropwise to the reaction liquid. After the dropwise addition was completed, the reaction process was monitored by TLC spot plate (ethyl acetate:petroleum ether=1:8). After the reaction was completed, to the reaction liquid were added saturated aqueous ammonium chloride solution (50 mL) and water (50 mL), and the mixture was extracted with ethyl acetate (400 mL). The organic phase was separated, and the aqueous phase was extracted once with ethyl acetate (200 mL). The organic phases were combined and concentrated to afford a crude, which was purified by column chromatography (eluent:ethyl acetate:petroleum ether=1:8), and then the eluent was concentrated, to afford target compound 3C (12.9 g, 66.54%).

LC-MS (ESI): m/z=334.2 [M+H]⁺.

### Step 3:

Compound 3C (12.9 g, 38.59 mmol) was weighed and dissolved in a mixed solution of anhydrous ethanol (260 mL) and water (40 mL), and then ammonium chloride (30.96 g, 578.85 mmol) and reduced iron powder (21.55 g, 385.9 mmol) were added. After the addition was completed, the mixture was heated to 80° C. and reacted for 2 h. After the reaction was completed, the reaction liquid was cooled to room temperature, to the reaction liquid were added saturated aqueous sodium bicarbonate solution and ethyl acetate (400 mL), and the mixture was filtered with diatomaceous earth. The filter cake was washed with ethyl acetate (100 mL), and the organic phase in the filtrate was separated, dried over anhydrous sodium sulfate and concentrated to afford a crude, which was slurried with a mixed solvent (30 mL, ethyl acetate:petroleum ether=10:1), filtered and dried, to afford target compound 3D (4.67 g, 46.86%).

LC-MS (ESI): m/z=259.1 [M+H]⁺.

¹H NMR (400 MHz, DMSO-d6) δ 12.03 (s, 1H), 8.87-8.86 (m, 1H), 8.14 (s, 1H), 7.48 (s, 1H), 4.40-4.35 (m, 2H), 2.23-2.16 (m, 1H), 1.37-1.33 (m, 3H), 1.05-1.01 (m, 2H), 0.92-0.90 (m, 2H).

### Step 4:

Compound 3D (4.6 g, 17.81 mmol) was weighed and dissolved in ultra-dry tetrahydrofuran (230 mL), and then the mixture was cooled to −60° C. with dry-ice ethanol. Lithium aluminum hydride (2.7 g, 71.24 mmol) was weighed and slowly added to the reaction liquid. After the addition was completed, the mixture was slowly warmed to about −40° C. and reacted for another 2 h. After the reaction was completed as monitored by LCMS, to the reaction liquid was slowly added a small amount of water (20 mL), the mixture was filtered to afford a solid, which was dried, heated to reflux with a mixed solution of dichloromethane and methanol (dichloromethane:MeOH=1:2, 300 mL), and filtered to afford a filtrate, and the filtrate was concentrated to afford an off-white solid. This process was repeated four times as such, and the resulting products were combined and concentrated, to afford crude target compound 3E (4.6 g), which was directly used in the subsequent reaction.

LC-MS (ESI): m/z=217.1 [M+H]⁺.

### Step 5:

Compound 3E (4.6 g, 21.27 mmol) was weighed and dissolved in a mixed solution of dichloromethane (70 mL) and tetrahydrofuran (70 mL), and then DMF (2 mL) was added. After the addition was completed, the mixture was cooled to below 0° C., and then thionyl chloride (9 mL) was slowly added. After the addition was completed, the mixture was kept at this temperature and reacted for 10 min. After the reaction was completed as monitored by TLC spot plate (dichloromethane:methanol=15:1), to the reaction liquid was added saturated aqueous sodium bicarbonate solution. After the addition was completed, the solution was alkaline (pH=8-9) and then was extracted with ethyl acetate (400 mL×3). The organic phases were combined, washed with saturated brine, dried over anhydrous sodium sulfate and concentrated, to afford crude target compound 3F (0.95 g, yield: 19.03%).

LC-MS (ESI): m/z=235.1 [M+H]⁺.

### Step 6:

Methyl 5-bromopyridine-2-carboxylate (0.7 g, 3.24 mmol), 3G (1.0 g, 3.24 mmol), chloro[2-(dicyclohexylphosphino)-3,6-dimethoxy-2′,4′,6′-triisopropyl-1,1′-biphenyl][2-(2-aminoethyl)phenyl]palladium (II) (RuPhos-Pd-G2, 0.26 mg, 0.32 mmol), and potassium carbonate (0.60 g, 4.44 mmol) were dissolved in anhydrous DMF (30 mL), and the mixture was reacted at 100° C. under nitrogen protection for 3 hours. After the reaction was completed as monitored by LCMS, the reaction liquid was spun to dryness to remove the solvent, purified by column chromatography (eluent:petroleum ether:ethyl acetate=1:3) and concentrated, to afford target compound 3H (1.00 g, yield: 97.08%).

LC-MS (ESI): m/z=319.2 [M+H]⁺.

### Step 7:

3H (0.3 g, 0.94 mmol) was weighed and dissolved in dioxane (5 ml) and water (1 mL), sodium hydroxide (0.5 g, 12.5 mmol) was added, and the mixture was stirred and reacted at 60° C. for 2 hours. After the reaction was completed as monitored by LCMS, 0.5M hydrochloric acid was added to adjust to pH=3, and the mixture was extracted twice with ethyl acetate and concentrated, to afford crude target compound 3I (0.30 g), which was directly used in the next reaction.

LC-MS (ESI): m/z=305.1 [M+H]⁺.

### Step 8:

31 (0.3 g, 0.99 mmol) was dissolved in N,N-dimethylformamide (10 mL), diisopropylethylamine (0.38 g, 2.97 mmol) and 2-(7-aza-1H-benzotriazol-1-yl)-1,1,3,3-tetramethyluronium hexafluorophosphate (0.75 g, 1.97 mmol) were then added, and the mixture was stirred at room temperature for half an hour. Cyclopropylamine (0.14 g, 2.46 mmol) was added, and the mixture was stirred for another 1 hour. Water was added for dilution, and the mixture was extracted three times with ethyl acetate (10 ml×3). The organic phase was washed with saturated brine (50 mL×3), dried over anhydrous sodium sulfate, concentrated and purified by column chromatography (eluent:ethyl acetate:petroleum ether=10:1), to afford target compound 3J (0.29 g, 85.20%).

LCMS m/z=344.3 [M+1]⁺

### Step 10:

Compound 3J (0.29 g, 0.85 mmol) was weighed and dissolved in dichloromethane (8 mL), a solution of hydrogen chloride in dioxane (1 mL, 4 mmol/L) was then added, and the mixture was stirred at room temperature for 20 h. After the reaction was completed, the reaction liquid was concentrated under reduced pressure, to afford target compound 3K hydrochloride (0.27 g), which was directly used in the next reaction. LCMS m/z=244.1 [M+1]⁺

### Step 11:

Compound 3K hydrochloride (0.10 g, 0.36 mmol) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford target compound 3 (0.048 g, 31.54%).

LC-MS (ESI): m/z=442.50 [M+H]⁺.

¹H NMR (400 MHz, DMSO-d₆) δ 11.84 (s, 1H), 8.66-8.63 (m, 2H), 8.39 (s, 1H), 8.01-7.95 (m, 2H), 7.62 (s, 1H), 7.42 (s, 1H), 6.40 (s, 1H), 3.71 (s, 2H), 3.15-3.14 (m, 2H), 2.90-2.89 (m, 1H), 2.72-2.68 (m, 2H), 2.54 (s, 2H), 2.17-2.11 (m, 1H), 0.98-0.96 (m, 2H), 0.83-0.82 (m, 2H), 0.70-0.66 (m, 4H).

### Example 4

### 5-(4-((7-cyclopropyl-6-oxo-5,6-dihydro-1,5-naphthyridin-3-yl)methyl)piperazin-1-yl)-N-(cyclopropylmethyl)picolinamide (Compound 4)

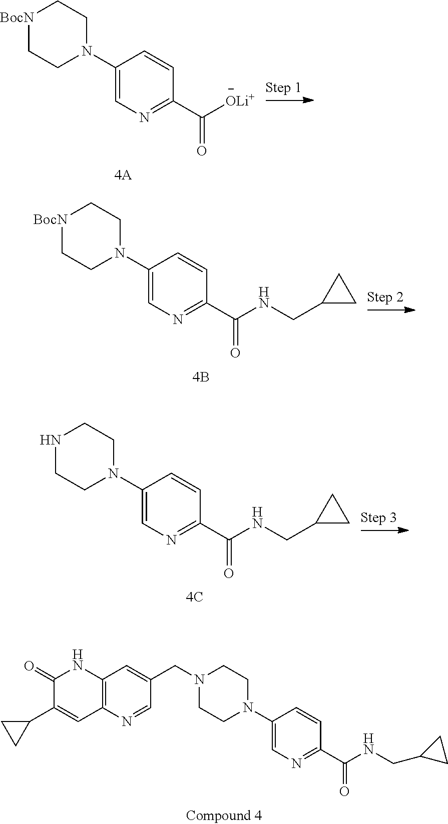

### Step 1:

Compound 4A (0.313 g, 1.0 mmol) (synthesized according to the synthesis method in patent US 2018141923A1) was weighed and dissolved in DMF (10 mL), HATU (570 mg, 1.5 mmol) was then added under stirring, and the mixture was stirred at room temperature. After the solid was completely dissolved, DIEPA (1 mL) was added, cyclopropylmethylamine (0.14 g, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 4B (0.332 g, 92.2%).

LC-MS (ESI): m/z=361.2 [M+H]⁺.

### Step 2:

4B (0.332 g, 0.92 mmol) was dissolved in methanol (5 mL), and hydrogen chloride/1,4-dioxane (5 mL, 4M) solution was added. The mixture was reacted at room temperature for 4 hours and spun to dryness, to afford target compound 4C (0.28 g, crude), which was directly used in the subsequent reaction.

LC-MS (ESI): m/z=261.2 [M+H]⁺.

### Step 3:

Compound 4C (0.10 g, 0.35 mmol) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford target compound 4 (0.047 g, 29.01%).

LC-MS (ESI): m/z=459.70 [M+H]⁺.

¹H NMR (400 MHz, DMSO-d₆) δ 11.86 (s, 1H), 8.43-8.38 (m, 2H), 8.28-8.27 (m, 1H), 7.85-7.83 (m, 1H), 7.61 (s, 1H), 7.42-7.38 (m, 2H), 3.64 (s, 2H), 3.34-3.33 (m, 4H), 3.15-3.12 (m, 2H), 2.57-2.55 (m, 4H), 2.16-2.12 (m, 1H), 1.05-1.01 (m, 1H), 0.99-0.95 (m, 2H), 0.84-0.80 (m, 2H), 0.43-0.38 (m, 2H), 0.24-0.22 (m, 2H).

### Example 5

### N-cyclopropyl-5-(4-((7-cyclopropyl-6-oxo-5,6-dihydro-1,5-naphthyridin-3-yl)methyl)piperazin-1-yl)picolinamide (Compound 5)

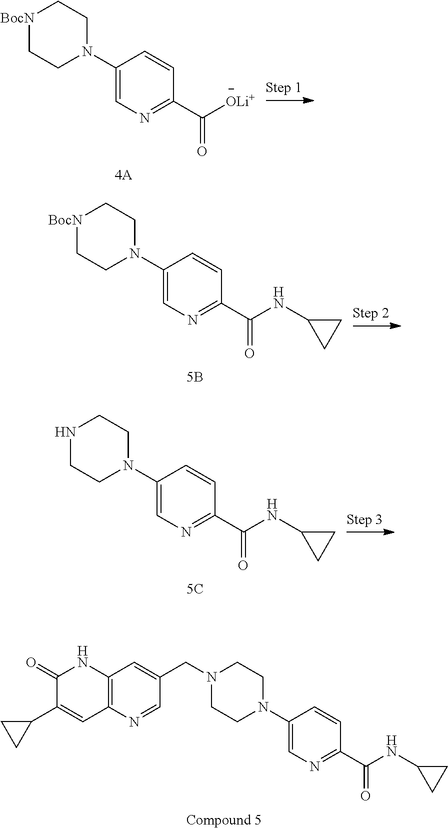

### Step 1:

Compound 4A (0.313 g, 1.0 mmol) was weighed and dissolved in DMF (10 mL), HATU (570 mg, 1.5 mmol) was then added under stirring, and the mixture was stirred at room temperature. After the solid was completely dissolved, DIEPA (1 mL) was added, cyclopropylamine (0.12 g, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 5B (0.309 g, 89.2%).

LC-MS (ESI): m/z=347.2 [M+H]⁺.

### Step 2:

5B (0.309 g, 0.89 mmol) was dissolved in methanol (5 mL), and hydrogen chloride/1,4-dioxane (5 mL, 4M) solution was added. The mixture was reacted at room temperature for 4 hours and spun to dryness, to afford target compound 5C (0.27 g, crude), which was directly used in the subsequent reaction.

LC-MS (ESI): m/z=247.2 [M+H]⁺.

### Step 3:

Compound 5C (0.10 g, crude) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford compound 5 (0.054 g, 37.2%).

LC-MS (ESI): m/z=445.1 [M+H]⁺.

¹H NMR (400 MHz, DMSO-d₆) δ 11.85 (s, 1H), 8.68-8.63 (m, 1H), 8.41-8.35 (m, 1H), 8.29-8.21 (m, 1H), 7.86-7.79 (m, 1H), 7.61 (s, 1H), 7.44-7.35 (m, 2H), 3.64 (s, 2H), 3.41-3.36 (m, 4H), 2.90-2.78 (m, 1H), 2.57-2.55 (m, 4H), 2.44-2.28 (m, 1H), 2.20-2.09 (m, 2H), 1.00-0.93 (m, 2H), 0.89-0.75 (m, 2H), 0.71-0.56 (m, 2H).

### Example 6

### 5-(4-((7-cyclopropyl-6-oxo-5,6-dihydro-1,5-naphthyridin-3-yl)methyl)piperazin-1-yl)-N-(1-methyl-1H-pyrazol-4-yl)picolinamide (Compound 6)

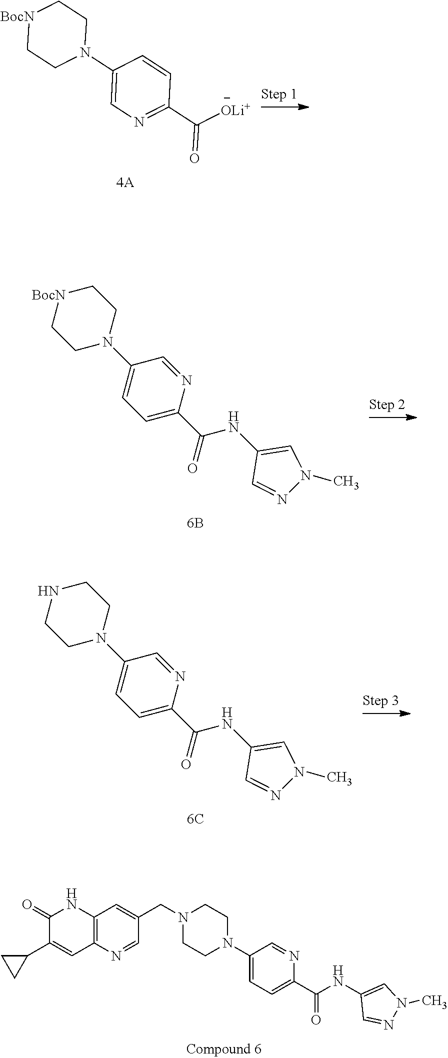

### Step 1:

Compound 4A (0.313 g, 1.0 mmol) was weighed and dissolved in DMF (10 mL), HATU (570 mg, 1.5 mmol) was then added under stirring, and the mixture was stirred at room temperature. After the solid was completely dissolved, DIEPA (1 mL) was added, 1-methyl-1H-pyrazol-4-amine (0.19 g, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 6B (0.342 g, 88.5%).

LC-MS (ESI): m/z=387.2 [M+H]⁺.

### Step 2:

6B (0.342 g, 0.88 mmol) was dissolved in methanol (5 mL), and hydrogen chloride/1,4-dioxane (5 mL, 4M) solution was added. The mixture was reacted at room temperature for 4 hours and spun to dryness, to afford target compound 6C (0.29 g, crude), which was directly used in the subsequent reaction.

LC-MS (ESI): m/z=287.2 [M+H]⁺.

### Step 3:

Compound 6C (0.12 g, crude) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford compound 6 (0.077 g, 48.1%).

LC-MS (ESI): m/z=485.2 [M+H]⁺.

¹H NMR (400 MHz, DMSO-d₆) δ 11.85 (s, 1H), 10.49 (s, 1H), 8.40-8.37 (m, 1H), 8.33-8.29 (m, 1H), 8.02 (s, 1H), 7.94-7.88 (m, 1H), 7.70-7.67 (m, 1H), 7.63-7.59 (m, 1H), 7.66-7.40 (m, 2H), 3.81 (s, 3H), 3.65 (s, 2H), 3.41-3.33 (m, 4H), 2.61-2.53 (m, 4H), 2.20-2.09 (m, 1H), 1.01-0.93 (m, 2H), 0.86-0.78 (m, 2H).

### Example 7

### 5-(4-((7-cyclopropyl-6-oxo-5,6-dihydro-1,5-naphthyridin-3-yl)methyl)piperazin-1-yl)-N-((3,3-difluorocyclobutyl)methyl)picolinamide (Compound 7)

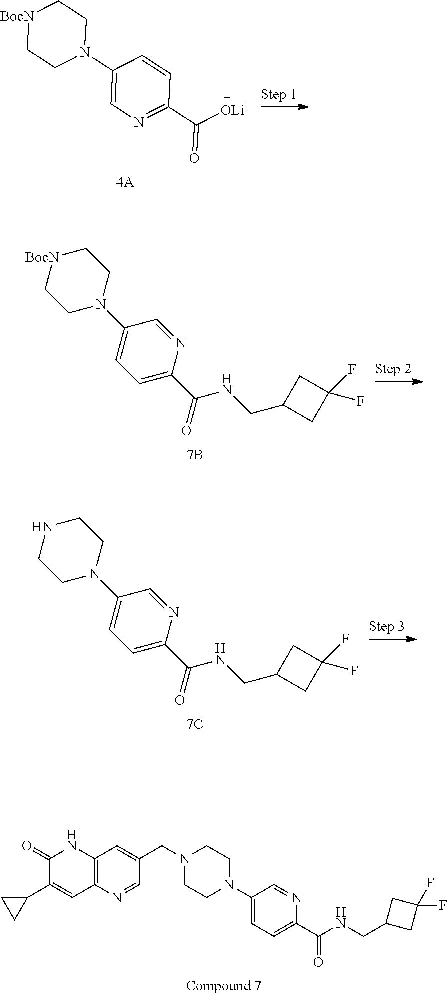

### Step 1:

Compound 4A (0.313 g, 1.0 mmol) was weighed and dissolved in DMF (10 mL), HATU (570 mg, 1.5 mmol) was then added under stirring, and the mixture was stirred at room temperature. After the solid was completely dissolved, DIEPA (1 mL) was added, 3,3-difluorocyclobutylmethylamine (0.24 g, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 7B (0.374 g, 91.3%).

LC-MS (ESI): m/z=411.2 [M+H]⁺.

### Step 2:

7B (0.374 g, 0.88 mmol) was dissolved in methanol (5 mL), and hydrogen chloride/1,4-dioxane (5 mL, 4M) solution was added. The mixture was reacted at room temperature for 4 hours and spun to dryness, to afford target compound 7C (0.31 g, crude), which was directly used in the subsequent reaction.

LC-MS (ESI): m/z=311.2 [M+H]⁺.

### Step 3:

Compound 7C (0.15 g, crude) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford compound 7 (0.067 g, 40.1%).

LC-MS (ESI): m/z=509.2 [M+H]⁺.

¹H NMR (400 MHz, DMSO-d₆) δ 11.85 (s, 1H), 8.68-8.60 (m, 1H), 8.39-8.36 (m, 1H), 8.28-8.25 (m, 1H), 7.86-7.81 (m, 1H), 7.63-7.59 (m, 1H), 7.47-7.32 (m, 2H), 3.64 (s, 2H), 3.43-3.31 (m, 6H), 2.64-2.52 (m, 6H), 2.45-2.28 (m, 3H), 2.19-2.10 (m, 1H), 1.01-0.94 (m, 2H), 0.86-0.79 (m, 2H).

### Example 8

### 3-cyclopropyl-7-((4-(2-(trifluoromethyl)imidazo[1,2-a]pyrazin-6-yl)piperazin-1-yl)methyl)-1,5-naphthyridin-2(1H)-one (Compound 8)

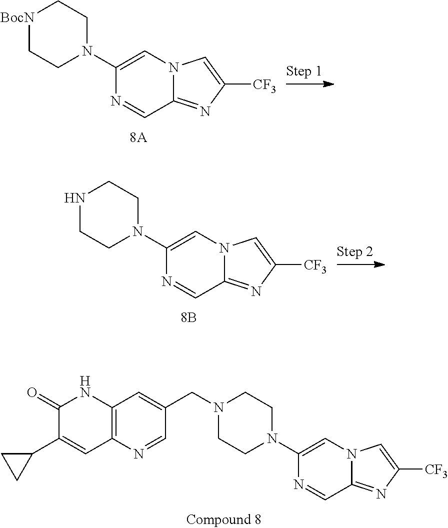 [SMILES: CC(C)(C)OC(=O)N1CCN(c2cn3cc(C(F)(F)F)nc3cn2)CC1 ; FC(F)(F)c1cn2cc(N3CCNCC3)ncc2n1 ; O=c1[nH]c2cc(CN3CCN(c4cn5cc(C(F)(F)F)nc5cn4)CC3)cnc2cc1C1CC1]

### Step 1:

8A (0.371 g, 1 mmol) was dissolved in methanol (5 mL), and hydrogen chloride/1,4-dioxane (5 mL, 4M) solution was added. The mixture was reacted at room temperature for 4 hours and spun to dryness, to afford target compound 8B (0.29 g, crude), which was directly used in the subsequent reaction.

LC-MS (ESI): m/z=272.2 [M+H]⁺.

### Step 2:

Compound 8B (0.15 g, crude) and compound 3F (0.076 g, 0.33 mmol) were weighed and dissolved in anhydrous DMF (5 mL), diisopropylethylamine (0.5 mL) and potassium iodide (0.12 g, 0.72 mmol) were then added, and the mixture was heated to 80° C., stirred and reacted for 1 h. After the reaction was completed as monitored by LCMS, to the reaction liquid were added water (10 mL) and ethyl acetate (50 mL). The organic phase was separated, and the aqueous phase was extracted twice with ethyl acetate (30 mL×2). The organic phases were combined, washed successively with water (50 mL) and saturated brine (50 mL), dried over anhydrous sodium sulfate, filtered and concentrated to afford a crude, which was purified by preparative TLC (developing agent: dichloromethane:methanol=10:1), to afford compound 8 (0.034 g, 21.9%).

LC-MS (ESI): m/z=470.1 [M+H]⁺.

¹H NMR (400 MHz, DMSO-d₆) δ 11.85 (s, 1H), 8.99 (s, 1H), 8.46 (s, 1H), 8.42-8.38 (m, 1H), 7.92 (s, 1H), 7.62 (s, 1H), 7.42 (s, 1H), 3.65 (s, 2H), 3.42-3.34 (m, 4H), 2.62-2.53 (m, 4H), 2.20-2.10 (m, 1H), 1.01-0.94 (m, 2H), 0.87-0.77 (m, 2H).

### Biological Test

### 1. PARP-1 Enzyme Activity Test Experiment

PARP-1 chemical fluorescence detection kit was purchased from BPS Bioscience. The histone solution in the kit was diluted 5× with 1×PBS, and 25 μL of the diluted histone solution was added to a microwell plate and incubated overnight at 4° C. After the incubation, the plate was washed three times with PBST (0.05% Tween-20). 100 μL of the blocking solution was added to the microwell plate and incubated at 25° C. for 90 minutes. After the incubation, the plate was washed three times with PBST. 2.5 μL of compounds at different concentrations diluted in test buffer and 12.5 μL of substrate mixed solution (1.25 μL 10× PARP test buffer; 1.25 μL 10× PARP test mixed solution; 2.5 μL Activated DNA, 7.5 μL double-distilled water) were added to the microwell plate. The PARP-1 enzyme was diluted to 2 ng/μL, 10 μL of the diluent was added to the microwell plate, and the reaction system was incubated at 25° C. for 60 minutes.

After the incubation, the plate was washed three times with PBST. Streptavidin-HRP was diluted 50× with a blocking solution, and 25 μL of the diluent was added to the microwell plate and incubated at 25° C. for 30 minutes. After the incubation, the plate was washed three times with PBST. ELISA ECL substrate A and substrate B were mixed at a ratio of 1:1 (v/v), 50 μL of the mixture was added to the microwell plate, and the chemiluminescence value was read.

The inhibition rate was calculated according to formula 1, where RLUsample was the readout of the compound well, RLUmax was the readout of the solvent control well, and RLUmin was the readout of the control well without the PARP-1 enzyme. Curve fitting was performed by four parameters (log (inhibitor) vs. response—Variable slope) using GraphPad Prism software, and the IC50 value was calculated.

$$( formula 1 )$$

$$Inhibition \% = ( 1 ( RLUsample - RLU min ) / \{ RLU max - RLU min ) ) \times 100 \%$$

Experimental results: the compound of the present invention had a significant inhibitory effect on PARP-1 enzyme activity in vitro, and the IC₅₀ value of the example compounds on PARP-1 enzyme activity was less than 100 μM. IC₅₀ values are represented by grades A, B, C, and D, where A represents IC₅₀≤1 nM, B represents IC₅₀≤10 nM, C represents IC₅₀≤50 nM, and D represents IC₅₀≤100 nM. The test results of some examples were shown in Table 1.

TABLE 1
PARP-1 enzyme activity

| Compound | IC₅₀ (nM) |
|---|---|
| 2 | A |
| 3 | A |
| 5 | A |
| 6 | A |

Conclusion: the compounds of the present invention have significant inhibitory effects on PARP-1 enzyme activity in vitro, especially compounds 2, 3, 5, 6 and 8, the IC₅₀ values of which on PARP-1 are 0.9 nM, 0.6 nM, 0.74 nM, 0.54 nM and 1.2 nM, respectively.

### 2. MDA-MB-436 Cell Activity Test Experiment

Breast tumor cells MDA-MB-436 were purchased from ATCC, the culture medium was Leibovitz's L-15+10% FBS, and the cells were cultured in a CO₂-free incubator at 37° C. On day 1, the cells in the exponential growth phase were collected, and the cell suspension was adjusted to 4000 cells/135 μL with the culture medium. 135 μL of the cell suspension was added to each well of a 96-well cell culture plate and incubated overnight. On day 2, compounds at different concentrations were added, and the plate was placed in the incubator and incubated for 7 days. After the incubation was completed, according to operation instructions for a CellTiter-Glo kit (Promega, G7573), 75 μL of CTG solution, which was already pre-melted and equilibrated to room temperature, was added to each well, and the mixture was uniformly mixed for 2 min using a microplate shaker. The plate was placed at room temperature for 10 min, and then fluorescence signal values were measured using an Envision2104 plate reader (PerkinElmer). The inhibition rate was calculated using formula (1), where RLU_{compound} was the readout of the drug treated group, RLU_{control} was the average value of the vehicle control group, and RLU_{blank} was the average value of the cell-free well. The IC₅₀ value was calculated using GraphPad Prism software.

$$( formula 1 )$$

$$Inh . \% = ( 1 - {RLU}_{compound} - {RLU}_{blank} ) / ( {RLU}_{control} - {RLU}_{blank} ) ) \times 100 \%$$

Test results: the compounds of the present invention had a significant inhibitory effect on MDA-MB-436 cells, the IC50 value of the compounds on MDA-MB-436 cells was less than 100 nM, the IC₅₀ value of some excellent compounds on MDA-MB-436 cells was less than 10 nM, and the IC₅₀ value of more excellent compounds on MDA-MB-436 cells was less than 1 nM; and the inhibitory rate of the compounds on breast tumor cells MDA-MB-436 was greater than 70%, the inhibition rate of some excellent compounds was greater than 85%, and the inhibition rate of more excellent compounds was greater than 90%. The results of some specific compounds were as shown in Table 2.

TABLE 2
MDA-MB-436 cell activity

| Compound | IC₅₀ (nM) | Max inh. % 10 μM |
|---|---|---|
| 2 | 5.1 | 86.3 |
| 3 | 4.5 | 82.1 |
| 5 | 5.6 | 80.7 |
| 6 | 2.8 | 85.4 |
| 8 | 1.1 | 97.4 |

Conclusion: the compound of the present invention has good inhibitory activity on breast tumor cells MDA-MB-436.

### 3. Pharmacokinetic Test in Mice

3.1 Experimental animals: male ICR mice, 20-25 g, 18 mice/compound. purchased from Chengdu Ddossy Experimental Animals Co., Ltd.
3.2 Experimental design: on the day of the test, 18 ICR mice were randomly grouped according to their body weights. the animals were fasted with water available for 12 to 14 h one day before the administration, and were fed 4 h after the administration.

TABLE 3
Administration information

| | | Administration information | | | | | |
|---|---|---|---|---|---|---|---|
| | | | | Administration | Administration | | |
| | Quantity | Test | Administration | concentration | volume | Collected | Mode of |
| Group | Male | compound | dosage (mg/kg) | (mg/mL) | (mL/kg) | samples | administration |
| G3 | 9 | Compound | 2.5 | 0.2 | 5 | Plasma | Intravenously |
| G4 | 9 | 3 | 10 | 1 | 10 | Plasma | Intragastrically |

Notes:
Solvent for intravenous administration: 5% DMA + 5% Solutol + 90% Saline; Solvent for intragastric administration: 5% DMSO + 30% PEG400 + 65% (20% SBE-CD)
(DMA: dimethylacetamide; Solutol: polyethylene glycol-15-hydroxystearate; Saline: physiological saline; MC: methylcellulose)

Before and after the administration, 0.06 mL of blood was taken from the orbit of the animals under isoflurane anesthesia and placed in an EDTAK2 centrifuge tube. The blood was centrifuged at 5000 rpm and 4° C. for 10 min to collect plasma. The blood collection time points for the intravenous group and intragastric administration group were: 0 min, 5 min, 15 min, 30 min, 1 h, 2 h, 4 h, 6 h, 8 h, and 24 h. Before analysis and detection, all samples were stored at −80° C., and a quantitative analysis of samples was performed using LC-MS/MS.

The test results were shown in Table 4.

TABLE 4
Pharmacokinetic parameters of test compounds in plasma of mice

| | | CL | | | |
|---|---|---|---|---|---|
| Test | Mode of | (mL/min/ | Vdₛₛ | AUC₀₋ₜ | F |
| compound | administration | kg) | (L/kg) | (hr*ng/mL) | (%) |
| Compound | i.v. (2.5 mg/kg) | 0.346 | 0.207 | 107581 | — |
| 3 | i.g. (10 mg/kg) | — | — | 707389 | 164 |

—: not applicable.

Conclusion: compound 3 has good pharmacokinetic characteristics in mice.

### 4. Pharmacokinetic Test in Rats

1.1 Experimental animals: male SD rats, about 220 g, 6-8 weeks old, 6 rats/compound, purchased from Chengdu Ddossy Experimental Animals Co., Ltd.
1.2 Experimental design: on the day of the test, 6 SD rats were randomly grouped according to their body weight; the animals were fasted with water available for 12 to 14 h one day before the administration, and were fed 4 h after the administration.

TABLE 5
Administration information

| | | Administration information | | | | | |
|---|---|---|---|---|---|---|---|
| | | | | Administration | Administration | | |
| | Quantity | Test | Administration | concentration | volume | Collected | Mode of |
| Group | Male | compound | dosage (mg/kg) | (mg/mL) | (mL/kg) | samples | administration |
| G1 | 3 | Compound | 2.5 | 0.5 | 5 | Plasma | Intravenously |
| G2 | 3 | 2 | 10 | 1 | 10 | Plasma | Intragastrically |

Notes:
Solvent for intravenous administration: 5% DMA + 5% Solutol + 90% Saline; Solvent for intragastric administration: 5% DMSO + 30% PEG400 + 65% (20% SBE-CD)
(DMA: dimethylacetamide; Solutol: polyethylene glycol-15-hydroxystearate; Saline: physiological saline; MC: methylcellulose)

Before and after the administration, 0.15 ml of blood was taken from the orbit of the animals under isoflurane anesthesia and placed in an EDTAK2 centrifuge tube. The blood was centrifuged at 5000 rpm and 4° C. for 10 min to collect plasma. The blood collection time points for the intravenous administration group and intragastric administration group were: 0, 5 min, 15 min, 30 min, 1 h, 2 h, 4 h, 6 h, 8 h, and 24 h. Before analysis and detection, all samples were stored at −80° C., and a quantitative analysis of samples was performed using LC-MS/MS.

The test results were shown in Table 6.

TABLE 6
Pharmacokinetic parameters of test compounds in plasma of rats

| | | CL | | | |
|---|---|---|---|---|---|
| Test | Mode of | (mL/min/ | Vdₛₛ | AUC₀₋ₜ | F |
| compound | administration | kg) | (L/kg) | (hr*ng/mL) | (%) |
| Compound | i.v. (2.5 mg/kg) | 0.585 | 0.169 | 70806 | — |
| 2 | i.g. (10 mg/kg) | — | — | 205512 | 72.6 |

—: not applicable.

Conclusion: compound 2 has good pharmacokinetic characteristics in rats.

1. A compound as shown in formula (I), or a stereoisomer, solvate, deuterated compound, or pharmaceutically acceptable salt thereof, 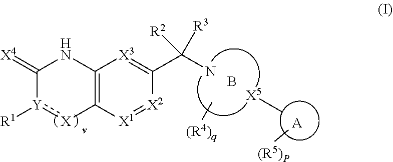
wherein X is selected from CR^{x}, C(R^{x})₂, O, N or NR^{x};
Y is selected from N, C or CH; [symbol]
represents a single bond or a double bond;
v is selected from 1, 2 or 3;
X¹, X² and X³ are each independently selected from N or CR^{x};
provided that when [symbol] represents a double bond, and v is selected from 1, X, X¹, X² and X³ are not all selected from CR^{x};
X⁴ is selected from O or S;
X⁵ is selected from N or CR^{x};
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1, 2 or 3;
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
each R⁴ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy;
or two R⁴ on the same carbon atom together with the carbon atom to which they are attached form ═O;
each R⁵ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy;
q is selected from 0, 1, 2 or 3; p is selected from 0, 1, 2 or 3;
ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms;
ring A is selected from 5- to 6-membered monocyclic heteroaromatic ring containing 1-5 nitrogen, oxygen or sulfur atoms, wherein the heteroaromatic ring is further substituted with 1-3 substituents selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b};
Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino,—NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy;
R_{b} is selected from —C(O)N(R^{a1})₂, —NR^{a1}C(O)OR^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl or deuterated C₁₋₆ alkoxy;
each R^{a1} is independently selected from H, D, C₁₋₆ alkyl, C₃₋₁₂ cycloalkyl, 3- to 12-membered heterocycloalkyl, 5- to 10-membered heteroaryl, C₁₋₆ alkoxy, C₁₋₆ alkyl-O—C₁₋₆ alkyl, C₁₋₆ alkyl-O—C₃₋₁₂ cycloalkyl, halo C₁₋₆ alkyl, halo C₁₋₆ alkoxy, deuterated C₁₋₆ alkyl, or deuterated C₁₋₆ alkoxy, wherein the alkyl, cycloalkyl, heterocycloalkyl, or heteroaryl is optionally further substituted with 1-3 substituents selected from D, halogen, cyano, hydroxyl, amino, C₁₋₆ alkyl, halo C₁₋₆ alkyl, deuterated C₁₋₆ alkyl, C₃₋₆ cycloalkyl or phenyl;
provided that:
(3) when [symbol] represents a single bond, X¹, X² and X³ are all selected from CR^{x}, and v is selected from 1, X is selected from C(R^{x})₂ or NR^{x};
(4) when 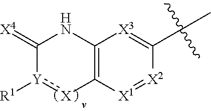
is selected from 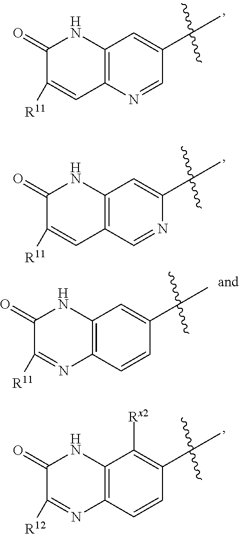
wherein R^{x2} is selected from H, C₁₋₆ alkyl or halogen, R¹² is selected from H, C₁₋₆ alkyl, halo C₁₋₆ alkyl, C₁₋₆ alkoxy or C₃₋₆ cycloalkyl, R¹¹ is selected from C₁₋₆ alkyl, and R² is selected from H or D, R³ is not selected from H or D;
unless otherwise specified, the above-mentioned heterocycloalkane, heterocycloalkyl, heteroaryl, or heteroaromatic ring contains 1-5 heteroatoms selected from nitrogen, oxygen or sulfur.

2. The compound, or the stereoisomer, solvate, deuterated compound, or pharmaceutically acceptable salt thereof according to claim 1, wherein R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl; provided that when R² is selected from H or D, R³ is not selected from H or D.

3. The compound, or the stereoisomer, solvate, deuterated compound, or pharmaceutically acceptable salt thereof according to claim 1, having a structure of formula (II) or (II-b): 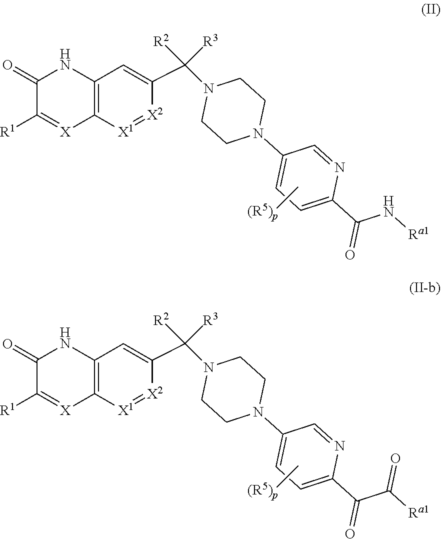
wherein X is selected from CRX or N;
X¹ and X² are each independently selected from N or CR^{x};
p is selected from 0 or 1;
R^{x} is independently selected from H, D, halogen, cyano, amino, hydroxyl, C₁₋₂ alkyl, C₁₋₂ alkoxy, C₂₋₄ alkenyl, C₂₋₄ alkynyl, C₁₋₂ alkyl-O—C₁₋₂ alkyl, —(CH₂)ᵣ—C₃₋₅ cycloalkyl, —(CH₂)ᵣ—C₅₋₉ bicyclic spiro cycloalkyl, —(CH₂)ᵣ-(4- to 5-membered heterocycloalkyl), or —(CH₂)ᵣ-(5- to 9-membered bicyclic spiro heterocycloalkyl);
R¹ is selected from C₁₋₆ alkyl or halo C₁₋₆ alkyl;
R⁵ is selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₄ cycloalkyl or phenyl;
R² and R³ are each independently selected from H, D, halogen, cyano, amino, hydroxyl, C₁₋₂ alkyl-O—C₁₋₂ alkyl, hydroxy C₁₋₃ alkyl, C₁₋₃ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form 3-membered cycloalkyl, 4-membered cycloalkyl, 5-membered cycloalkyl, 4-membered heterocycloalkyl, or 5-membered heterocycloalkyl, provided that when R² is selected from H or D, R³ is not selected from H or D.

4. The compound, or the stereoisomer, solvate, deuterated compound, or pharmaceutically acceptable salt thereof according to claim 1, having a structure of formula (III), (IV), (III-a), (III-b), (III-c), (IV-a), (IV-b) or (IV-c): 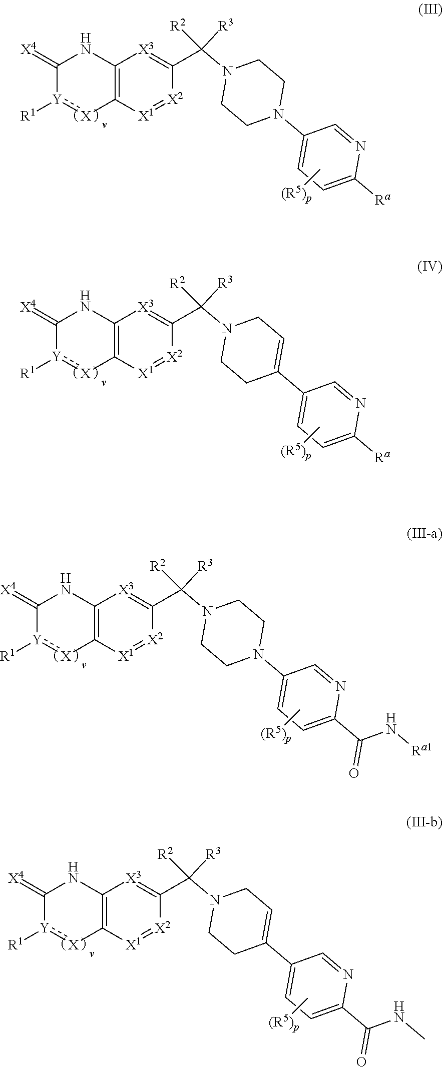 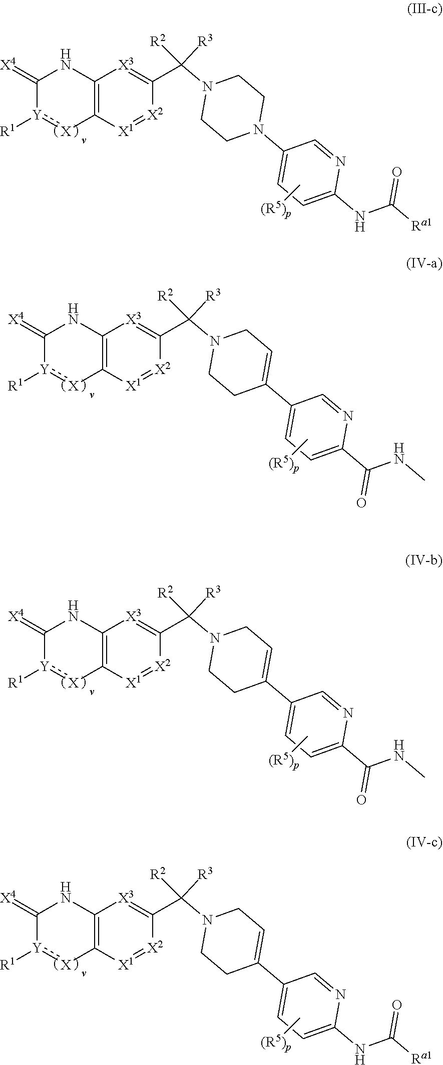
wherein X is selected from CR^{x}, C(R^{x})₂ or O;
Rₐ is selected from —C(O)N(R^{a1})₂, —NR^{a1}C(O)R^{a1}, —C(O)R^{a1}, —NR^{a1}, —NR^{a1}C(O)OR^{a1}, —NR^{a1}C(O)N(R^{a1})₂, —C(═S)N(R^{a1})₂, —S(O)₂N(R^{a1})₂, 5- to 6-membered monocyclic heteroaryl containing 1-4 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino,—NHC₁₋₂ alkyl, —N(C₁₋₂ alkyl)₂, C₁₋₄ alkyl, halo C₁₋₄ alkyl, C₁₋₄ alkoxy, halo C₁₋₄ alkoxy, deuterated C₁₋₄ alkyl, or deuterated C₁₋₄ alkoxy;
each R^{a1} is independently selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 10-membered heteroaryl, C₁₋₄ alkoxy, C₁₋₂ alkyl-O—C₁₋₂ alkyl, C₁₋₄ alkyl-O—C₃₋₆ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, F, Cl, cyano, hydroxyl, amino, C₁₋₂ alkyl, halo C₁₋₂ alkyl, deuterated C₁₋₂ alkyl, C₃₋₄ cycloalkyl or phenyl;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1 or 2;
provided that when R² is selected from H or D and R³ is selected from H or D, X⁴ is selected from O, X³ is selected from CH, [symbol] represents a double bond, v is selected from 1, and Y is selected from C, R¹ is not C₁₋₆ alkyl and halo C₁₋₆ alkyl.

5. The compound, or the stereoisomer, solvate, deuterated compound, or pharmaceutically acceptable salt thereof according to claim 4, wherein
X is selected from CR^{x}, C(R^{x})₂ or O;
v is selected from 1 or 2;
p is selected from 0 or 1;
each R^{x} is independently selected from H, D, halogen, cyano, amino, hydroxyl, C₁₋₄ alkyl, C₁₋₄ alkoxy, C₂₋₄ alkenyl, C₂₋₄ alkynyl, C₁₋₄ alkyl-O—C₁₋₄ alkyl, —(CH₂)ᵣ—C₃₋₆ monocyclic cycloalkyl or —(CH₂)ᵣ-(4- to 6-membered monocyclic heterocycloalkyl); or two R^{x} on the same carbon atom together form ═O;
each R⁵ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy;
R^{a1} is selected from C₁₋₄ alkyl, C₃₋₅ cycloalkyl, C₅₋₉ bicyclic spiro cycloalkyl, 4- to 6-membered heterocycloalkyl, 5- to 9-membered bicyclic spiro heterocycloalkyl, 5- to 6-membered heteroaryl, C₁₋₄ alkoxy, C₁₋₂ alkyl-O—C₁₋₂ alkyl, C₁₋₂ alkyl-O—C₃₋₅ cycloalkyl, halo C₁₋₄ alkyl, halo C₁₋₄ alkoxy, deuterated C₁₋₄ alkyl, or deuterated C₁₋₄ alkoxy, wherein the alkyl, cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, C₃₋₅ cycloalkyl or phenyl;
R² and R³ are each independently selected from H, D, halogen, cyano, amino, hydroxyl, C₁₋₂ alkyl-O—C₁₋₂ alkyl, hydroxy C₁₋₃ alkyl, C₁₋₃ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form 3-membered cycloalkyl, 4-membered cycloalkyl, 5-membered cycloalkyl, 4-membered heterocycloalkyl, or 5-membered heterocycloalkyl.

6. The compound, or the stereoisomer, solvate, deuterated compound, or pharmaceutically acceptable salt thereof according to claim 4, wherein R¹ is selected from halogen, nitro, cyano, amino, hydroxyl, —SF₅, C₁₋₄ alkyl, C₁₋₄ alkoxy, C₂₋₄ alkenyl, C₂₋₄ alkynyl, C₁₋₄ alkyl-O—C₁₋₄ alkyl, —(CH₂)ᵣ—C₃₋₆ monocyclic cycloalkyl, —(CH₂)-C₅₋₉ bicyclic spiro cycloalkyl, —(CH₂)ᵣ-(4- to 6-membered monocyclic heterocycloalkyl) or —(CH₂)ᵣ-(5- to 9-membered bicyclic spiro heterocycloalkyl), wherein the alkyl, alkoxy, alkenyl, alkynyl, cycloalkyl, or heterocycloalkyl is optionally further substituted with 1-3 groups selected from D, F, Cl, cyano, amino, hydroxyl, C₁₋₃ alkyl or C₁₋₃ alkoxy.

7. The compound, or the stereoisomer, solvate, deuterated compound, or pharmaceutically acceptable salt thereof according to claim 1, wherein 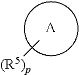
is selected from 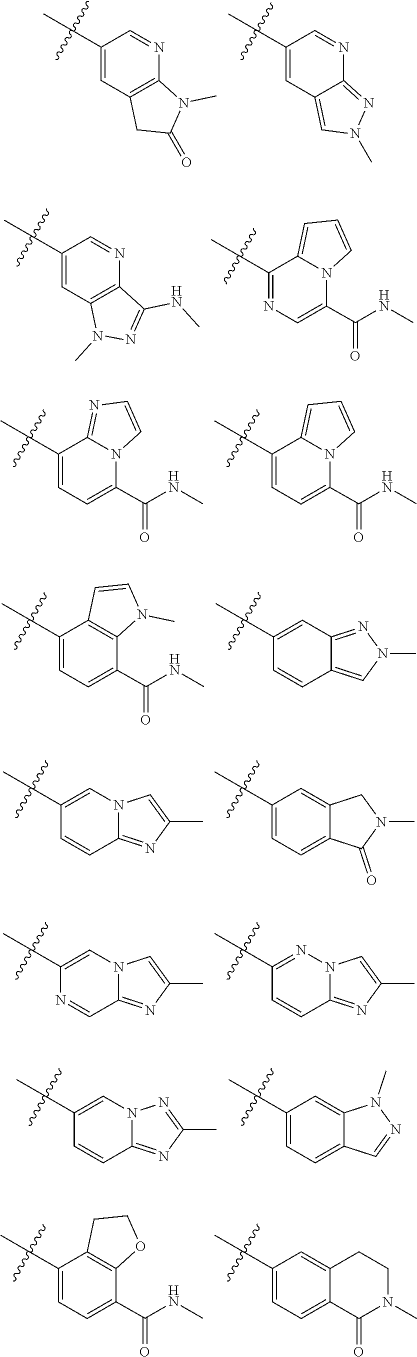 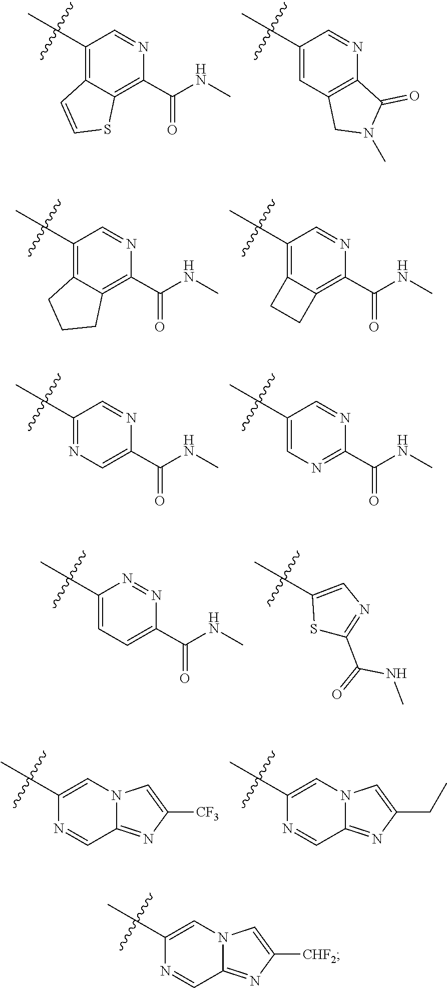
or 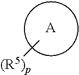
is selected from

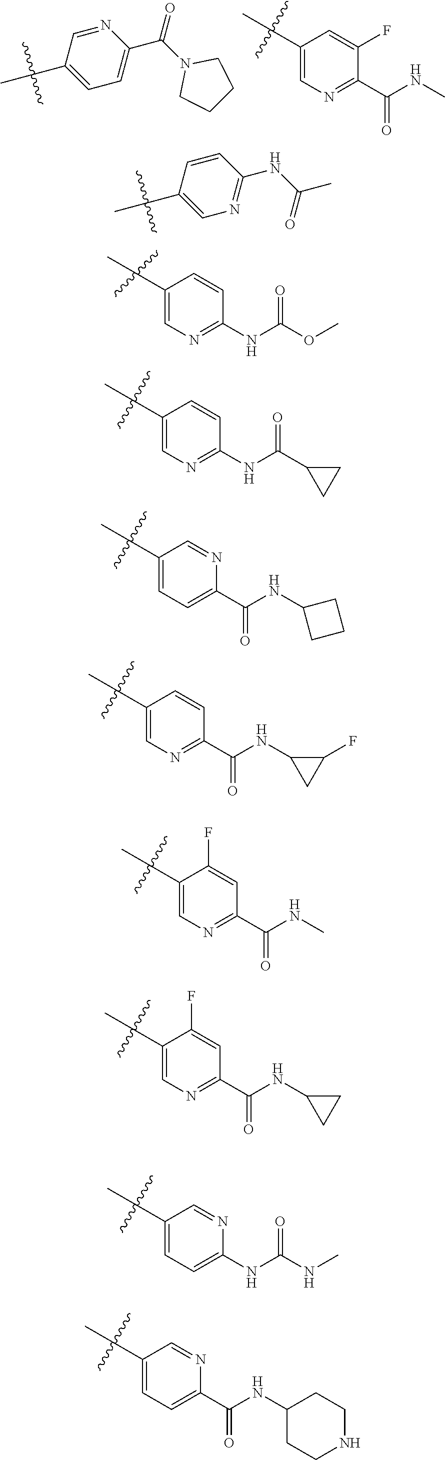

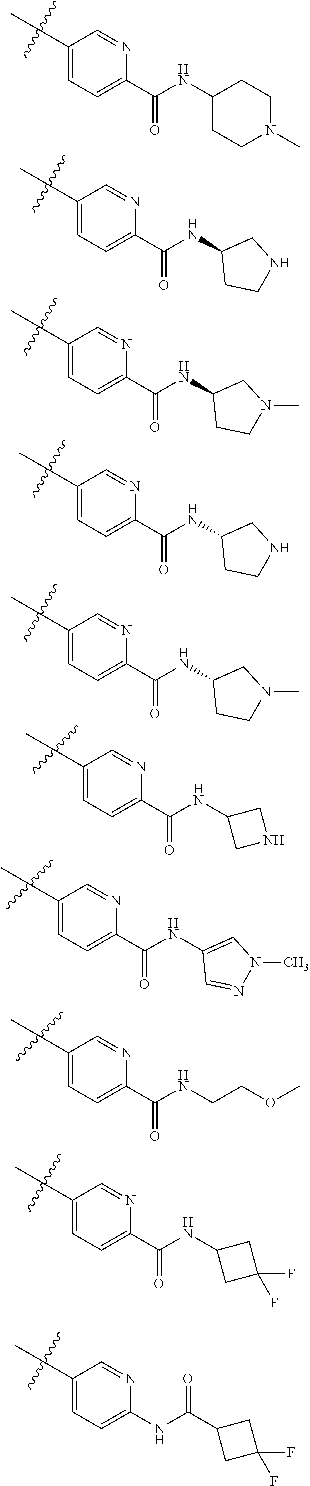

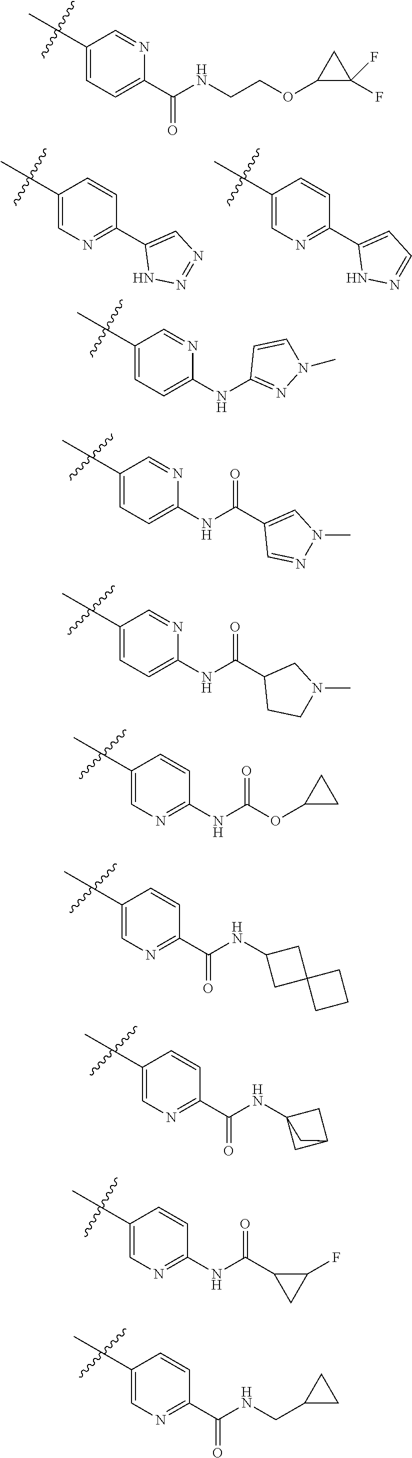

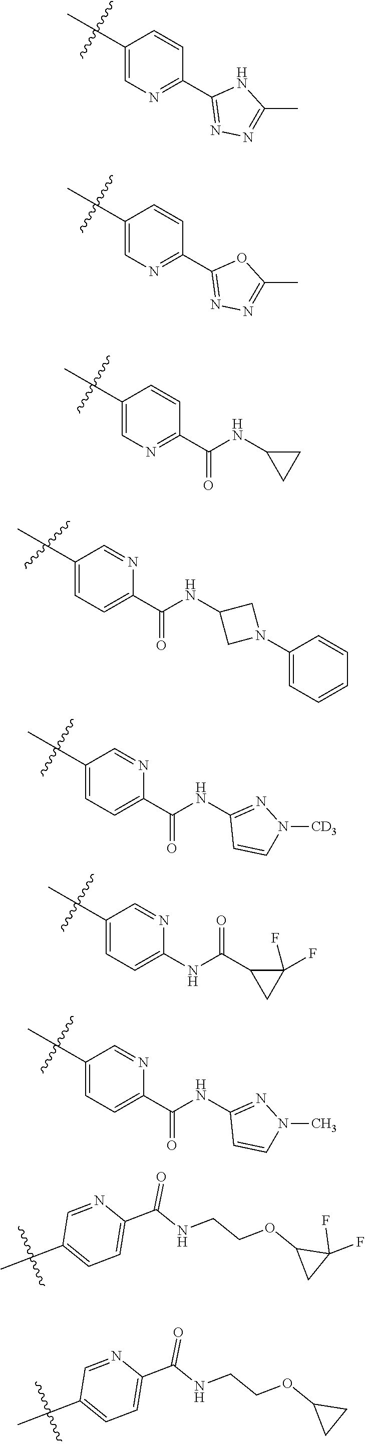

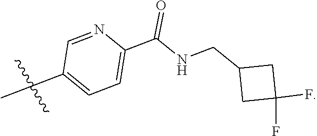 [SMILES: *c1ccc(C(=O)NCC2CC(F)(F)C2)nc1]

8. The compound, or the stereoisomer, solvate, deuterated compound, or pharmaceutically acceptable salt thereof according to claim 1, wherein ring B is selected from piperazinyl, piperidyl or

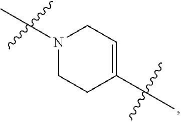 [SMILES: *C1=CCN(*)CC1]

9. The compound, or the stereoisomer, solvate, deuterated compound, or pharmaceutically acceptable salt thereof according to claim 1, wherein 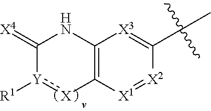
is selected from

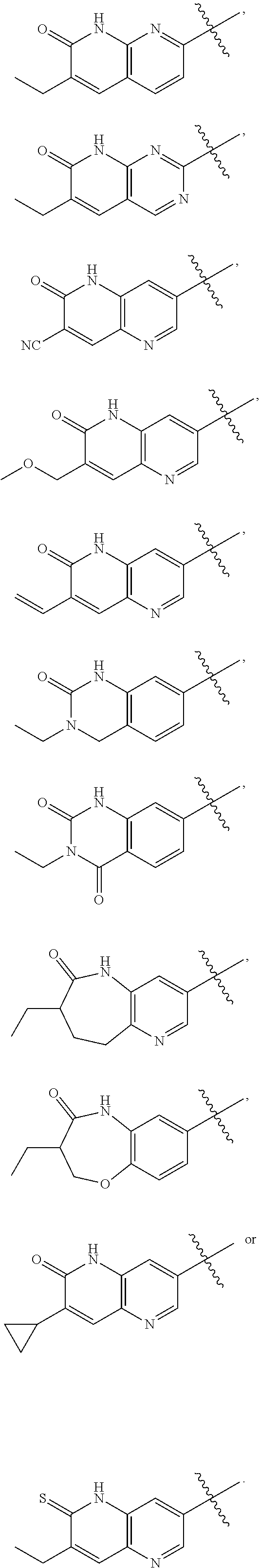

10. The compound, or the stereoisomer, solvate, deuterated compound, or pharmaceutically acceptable salt thereof according to claim 1, having a structure of formula (V): 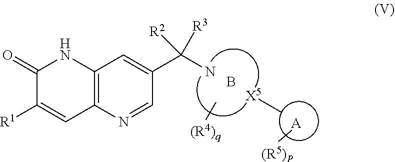
wherein R² and R³ are each independently selected from H, D, halogen, cyano, amino, hydroxyl, C₁₋₂ alkyl-O—C₁₋₂ alkyl, hydroxy C₁₋₂ alkyl, C₁₋₂ alkoxy, halo C₁₋₂ alkyl, halo C₁₋₂ alkoxy, deuterated C₁₋₂ alkyl, deuterated C₁₋₂ alkoxy or C₁₋₂ alkyl; or R² and R³ together with the carbon atom to which they are attached form C₃₋₅ cycloalkyl or 4- to 5-membered heterocycloalkyl;
each R⁴ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy;
or two R⁴ on the same carbon atom together with the carbon atom to which they are attached form ═O;
each R⁵ is independently selected from D, halogen, cyano, amino, hydroxyl, —SF₅, C₁₋₂ alkyl, C₁₋₂ alkoxy, halo C₁₋₂ alkyl, halo C₁₋₂ alkoxy, deuterated C₁₋₂ alkyl or deuterated C₁₋₂ alkoxy;
q is selected from 0 or 1; p is selected from 0 or 1;
ring B is selected from 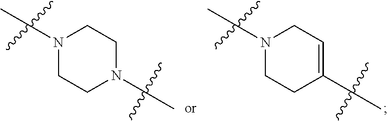 [SMILES: *N1CCN(*)CC1 ; *C1=CCN(*)CC1]
ring A is selected from 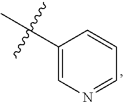 [SMILES: *c1cccnc1]
which is further substituted with 1-3 substituents selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b};
Rₐ is selected from —C(O)N(R^{a1})₂, —NR^{a1}C(O)R^{a1} or —C(O)R^{a1};
R_{b} is selected from D, halogen, cyano, hydroxyl, amino, C₁₋₂ alkyl, C₃₋₅ cycloalkyl, 3- to 5-membered heterocycloalkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl, or deuterated C₁₋₂ alkoxy;
each R^{a1} is independently selected from H, D, C₁₋₂ alkyl, C₃₋₅ cycloalkyl, 3- to 5-membered heterocycloalkyl, 5- to 6-membered heteroaryl, halo C₁₋₂ alkyl, halo C₁₋₂ alkoxy, deuterated C₁₋₂ alkyl, or deuterated C₁₋₂ alkoxy, wherein the cycloalkyl, heterocycloalkyl, or heteroaryl is optionally further substituted with 1-3 substituents selected from D, halogen, cyano, hydroxyl, amino, C₁₋₂ alkyl, halo C₁₋₂ alkyl, deuterated C₁₋₂ alkyl, or C₃₋₄ cycloalkyl.

11. The compound, or the stereoisomer, solvate, deuterated compound, or pharmaceutically acceptable salt thereof according to claim 1, having a structure of formula (VI), (VII), (VIII) or (IX): 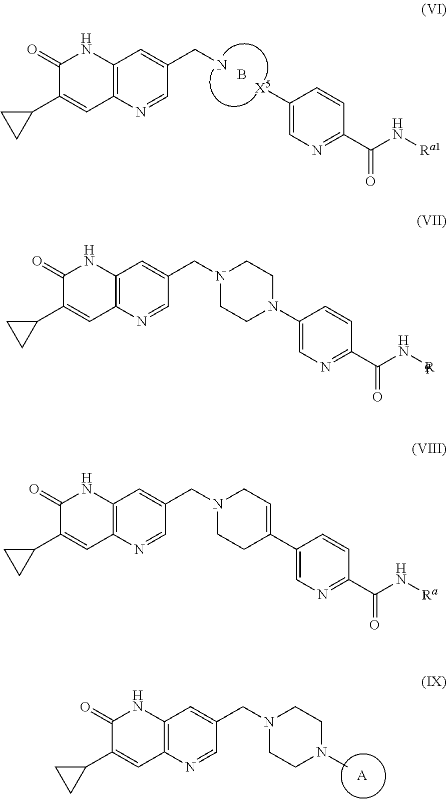
wherein ring B is selected from 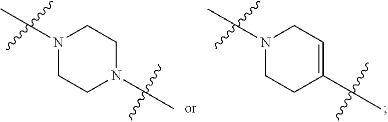 [SMILES: *N1CCN(*)CC1 ; *C1=CCN(*)CC1]
X⁵ is selected from N or CH;
each R^{a1} is independently selected from C₁₋₄ alkyl, C₃₋₅ cycloalkyl, or 5- to 6-membered heteroaryl, wherein the alkyl, cycloalkyl, or heteroaryl is optionally further substituted with 1, 2 or 3 substituents selected from D, F, Cl, cyano, hydroxyl, amino, C₁₋₂ alkyl, halo C₁₋₂ alkyl, or deuterated C₁₋₂ alkyl;
ring A is selected from 8- to 10-membered bicyclic heteroaromatic ring containing 1-5 nitrogen, oxygen or sulfur atoms, wherein the heteroaromatic ring is optionally further substituted with 1, 2 or 3 substituents selected from —C(O)N(R^{a1})₂, —NHC₁₋₂ alkyl, —N(C₁₋₂ alkyl)₂, C₁₋₂ alkyl, ═O, C₁₋₂ alkyl, C₃₋₅ cycloalkyl, halo C₁₋₂ alkyl, halo C₁₋₂ alkoxy, deuterated C₁₋₂ alkyl, or deuterated C₁₋₂ alkoxy.

12. The compound, or the stereoisomer, solvate, deuterated compound, or pharmaceutically acceptable salt thereof according to claim 1, wherein the compound is selected from one of the following structures:

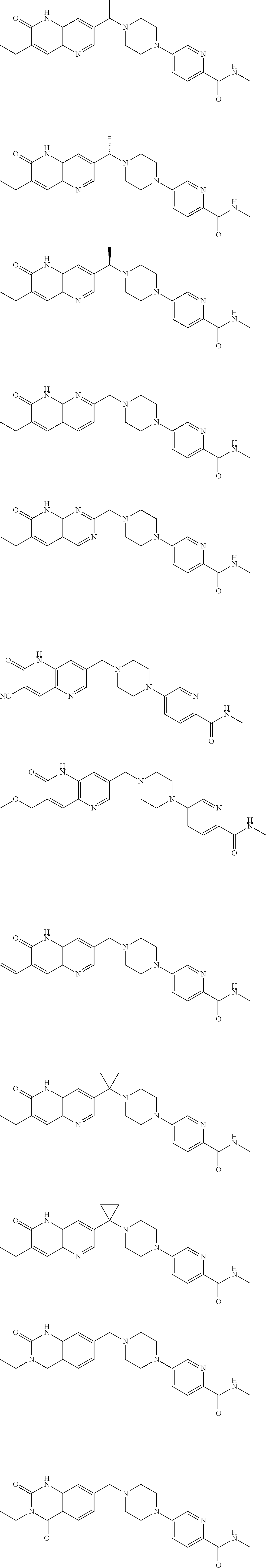

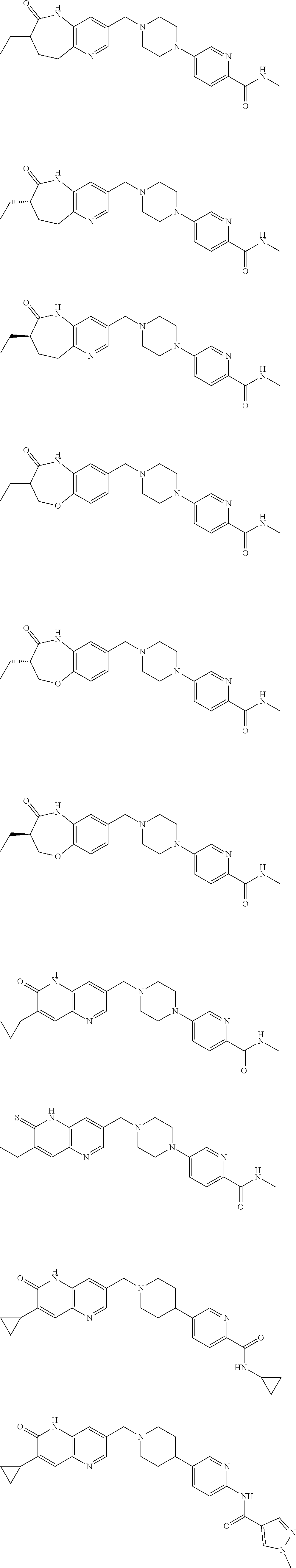

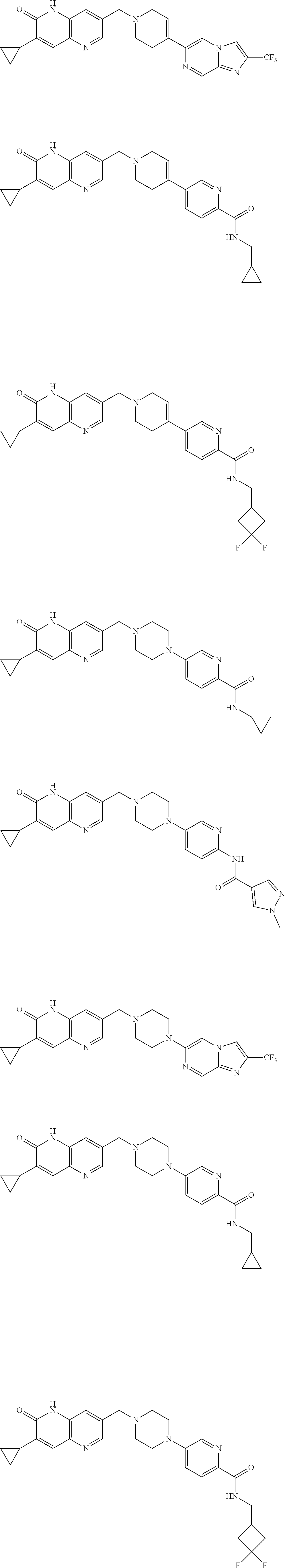

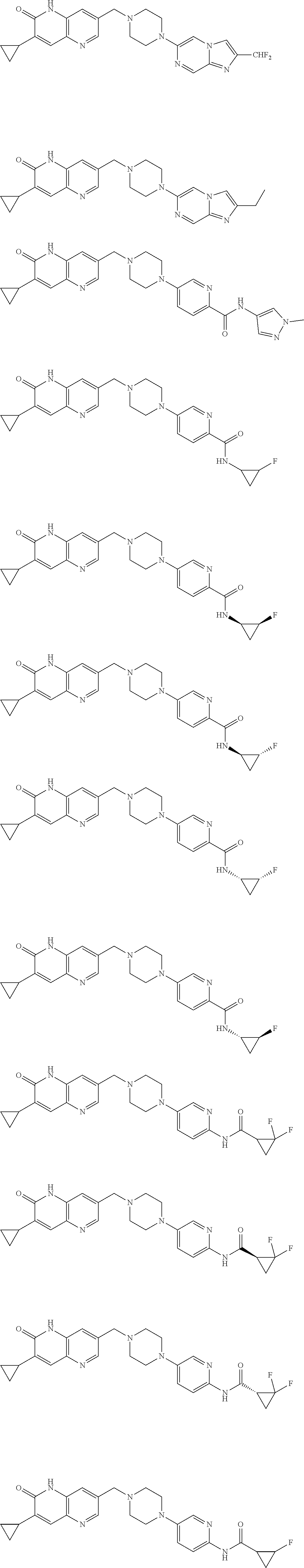

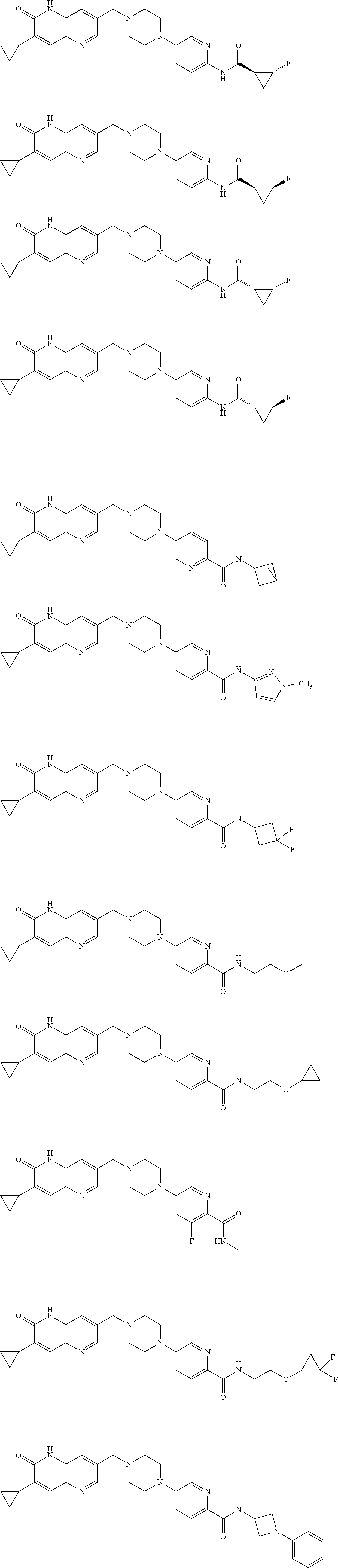

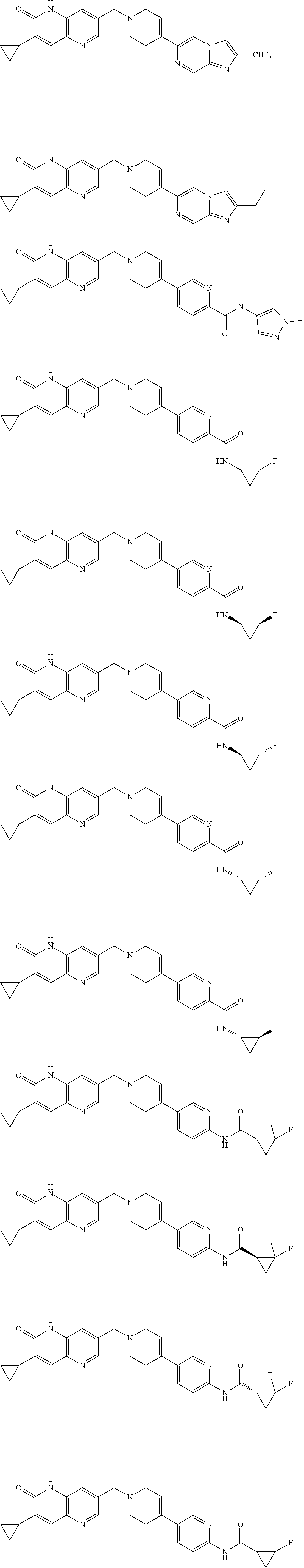

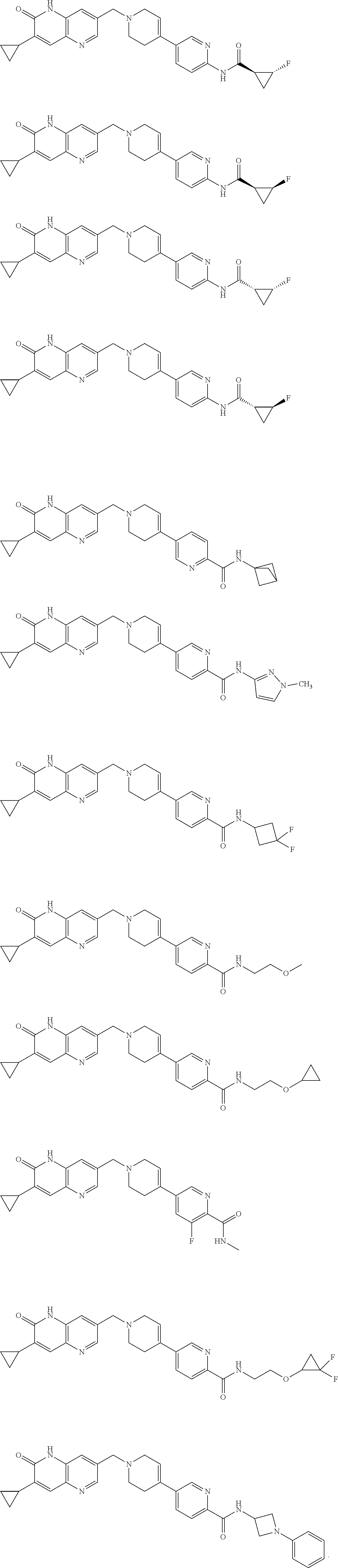

13. A pharmaceutical composition, wherein the pharmaceutical composition comprises the compound, or the stereoisomer, solvate, deuterated compound, or pharmaceutically acceptable salt thereof according to claim 1, and a pharmaceutically acceptable excipient and/or carrier.

14. (canceled)

15. (canceled)

16. A pharmaceutical composition or pharmaceutical preparation, comprising 1-1440 mg of the compound, or the stereoisomer, deuterated compound, solvate, or pharmaceutically acceptable salt thereof according to claim 1, and a carrier and/or excipient.

17. A method for treating a disease in a mammal, comprising administering to a subject a therapeutically effective amount of the compound, or the stereoisomer, deuterated compound, solvate, or pharmaceutically acceptable salt thereof according to claim 1, wherein the therapeutically effective amount is preferably 1-1440 mg, and the disease is preferably cancer.
